(19) United States
(12) Patent Application Publication
(10) Pub. No.: US 2024/0139175 A1
(43) Pub. Date: May 2, 2024

(54) COMBINATION THERAPY WITH A VINCA ALKALOID N-OXIDE AND AN IMMUNE CHECKPOINT INHIBITOR

(71) Applicant: CASCADE PRODRUG INC., Eugene, OR (US)

(72) Inventor: August J. SICK, San Tan Valley, AZ (US)

(73) Assignee: CASCADE PRODRUG INC., Eugene, OR (US)

(21) Appl. No.: 18/269,526

(22) PCT Filed: Dec. 23, 2021
(86) PCT No.: PCT/US2021/065059

§ 371 Date: Jun. 23, 2023

Related U.S. Application Data
(60) Provisional application No. 63/129,911, filed on Dec. 23, 2020.

(51) Int. Cl. A61K 31/475; A61K 9/127; A61K 9/19; A61P 35/00; C07K 16/28; A61K 39/00

(52) U.S. Cl. CPC A61K 31/475; A61K 9/127; A61K 9/19; A61P 35/00; C07K 16/2818; C07K 16/2827; A61K 2039/505

(57) ABSTRACT

The present disclosure provides therapeutic methods of treating a cancer patient with a vinca alkaloide N-oxide and an immune checkpoint inhibitor.

### BACKGROUND OF THE INVENTION

### Field of the Invention

The present disclosure provides therapeutic methods of treating a cancer patient with a vinca alkaloid N-oxide and an immune checkpoint inhibitor.

### Background

Vinca alkaloids are a class of chemotherapeutic agents originally discovered in the Madagascar periwinkle. Representative vinca alkaloids include vinblastine, vincristine, vindesine, vinorelbine, and vinflunine. N-oxides of vinca alkaloids function as prodrugs that are activated under the hypoxic conditions found in cancer tumors and other hypoxic environments. See U.S. Pat. Nos. 8,048,872 and 8,883,775.

Hypoxia is a common phenomenon in solid neoplasms. It arises when tissue oxygen demands exceed the oxygen supply from the vasculature. Hypoxic regions develop within solid tumors due to aberrant blood vessel formation, fluctuations in blood flow, and increasing oxygen demands from rapid tumor expansion. Hypoxia may limit tumor cell response to radiation, chemotherapy, and/or immunotherapy. Le and Courter, Cancer Metastasis Rev. 27:351-362 (2008). Thus, new combination therapies are needed to overcome hypoxia-mediated resistance to current cancer therapies. In particular, new combination therapies are needed to overcome resistance to cancer immunotherapies. Sharma et al., Cell 168(4): 707-723 (2017).

### BRIEF SUMMARY OF THE INVENTION

In one aspect, the present disclosure provides therapeutic methods of treating a cancer patient, the methods comprising administering to the patient therapeutically effective amounts of a vinca alkaloid N-oxide, e.g., vinblastine N_{b′}-oxide, vincristine N_{b′}-oxide, vindesine N_{b′}-oxide, vinorelbine N_{b′}-oxide, or vinflunine N_{b′}-oxide, and an immune checkpoint inhibitor, e.g., a PD-1 inhibitor, a PD-L1 inhibitor, a CTLA-4 inhibitor, a LAG3 inhibitor, a TIM3 inhibitor, a VISTA inhibitor, a TIGIT inhibitor, or a cd47 inhibitor.

In another aspect, the present disclosure provides therapeutic methods of treating a cancer patient, the methods comprising administering to the patient therapeutically effective amounts of a vinca alkaloid N-oxide and an immune checkpoint inhibitor, wherein one or more cancer biomarker proteins or genes is differentially present in a biological sample taken from the patient as compared with a biological sample taken from a subject of another phenotypic status.

In another aspect, the present disclosure provides kits comprising a vinca alkaloid N-oxide and an immune checkpoint inhibitor.

In another aspect, the present disclosure provides lyophilized pharmaceutical compositions comprising a vinca alkaloid N-oxide, or a pharmaceutically acceptable salt, encapsulated in a liposome.

In another aspect, the present disclosure provides kits comprising lyophilized pharmaceutical compositions comprising a vinca alkaloid N-oxide, or a pharmaceutically acceptable salt, encapsulated in a liposome, and an immune checkpoint inhibitor.

### DETAILED DESCRIPTION OF DRAWINGS

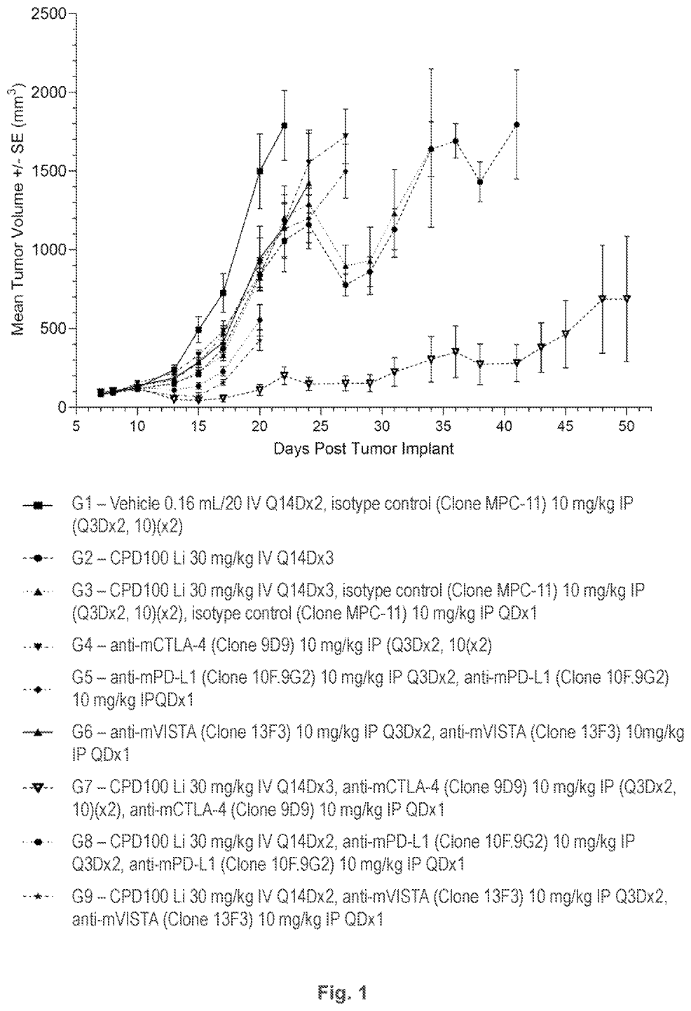

FIG. 1 is a line graph showing the mean tumor volume of Group 1-9 treated animals in the CT26.WT murine colon carcinoma model.

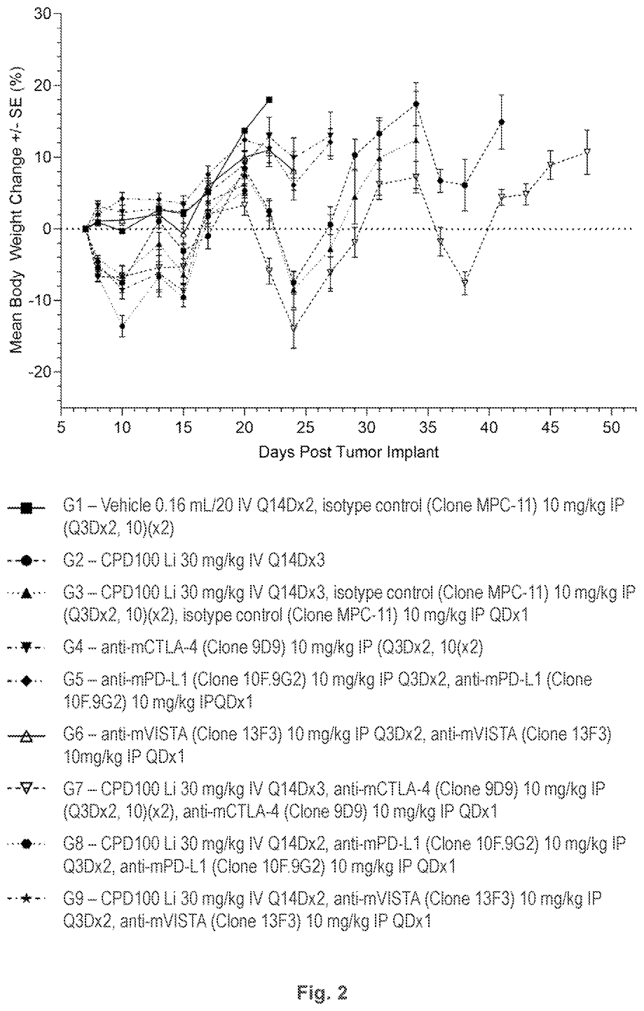

FIG. 2 is a line graph showing the mean body weight change in Group 1-9 treated animals in the CT26.WT murine colon carcinoma model.

### DETAILED DESCRIPTION OF THE INVENTION

In one embodiment, the present disclosure provides therapeutic methods of treating a patient having cancer, the method comprising administering to the patient a therapeutically effective amount of a vinca alkaloid N-oxide, e.g., vinblastine N_{b′}-oxide, vincristine N_{b′}-oxide, vindesine N_{b′}-oxide, vinorelbine N_{b′}-oxide, or vinflunine N_{b′}-oxide, and an immune checkpoint inhibitor, e.g., a PD-1 inhibitor, a PD-L1 inhibitor, a CTLA-4 inhibitor, a LAG3 inhibitor, a TIM3 inhibitor, a VISTA inhibitor, a TIGIT inhibitor, or a cd47 inhibitor.

In another embodiment, the present disclosure provides therapeutic methods of treating a patient having cancer, the method comprising administering to the patient a therapeutically effective amount of a vinca alkaloid N-oxide and an immune checkpoint inhibitor, wherein one or more of the genes listed in Table 1, see below, is differentially present in a biological sample taken from the patient as compared with a biological sample taken from a subject of another phenotypic status. In another embodiment, HIF overexpression is differentially present in a sample taken from the patient.

In another embodiment, a vinca alkaloid N-oxide is administered to the patient before the immune checkpoint inhibitor.

In another embodiment, a vinca alkaloid N-oxide is administered to the patient after the immune checkpoint inhibitor.

In another embodiment, a vinca alkaloid N-oxide is administered to the patient at the same time as an immune checkpoint inhibitor.

In another embodiment, the present disclosure provides kits comprising a vinca alkaloid N-oxide and an immune checkpoint inhibitor, and instructions for administering a vinca alkaloid N-oxide and the immune checkpoint inhibitor to a patient having cancer.

In another embodiment, the kit is packaged in a manner that facilitates its use to practice methods of the present disclosure.

In another embodiment, the kit includes a vinca alkaloid N-oxide (or a composition comprising a vinca alkaloid N-oxide) packaged in a container, such as a sealed bottle or vessel, with a label affixed to the container or included in the kit that describes use of a vinca alkaloid N-oxide or composition to practice the method of the disclosure. In one embodiment, a vinca alkaloid N-oxide is packaged in a unit dosage form. The kit further can include a device suitable for administering the composition according to the intended route of administration.

The disclosure provides various therapeutic methods, kits, and compositions relating to the treatment of cancer. In one embodiment, the cancer is a solid tumor. In another embodiment, the cancer is a hematological malignancy. In another embodiment, the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In another embodiment, the cancer is selected from the group consisting of squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, and squamous cell carcinoma of the skin.

In another embodiment, a precancerous tumor is selected from the group consisting of leukoplakia of the head and neck, Barrett's esophagus, metaplasia of the stomach, adenoma of the colon, chronic hepatitis, bile duct hyperplasia, pancreatic intraepithelial neoplasia, atypical adenomatous hyperplasia of the lungs, dysplasia of the bladder, cervical initraepithelial neoplasia, penile intraepithelial neoplasia, and actinic keratosis of the skin.

In another embodiment, the patient has tumors that overexpress HIF. The tumors may be determined to overexpress HIF by methods known in the art.

In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the cancer is selected from the group consisting of glioblastoma, hepatocellular carcinoma, non-small cell and small-cell lung cancer, head and neck cancer, colorectal carcinoma, and triple-negative breast cancer.

In another embodiment, the cancer has become resistant to conventional cancer treatments. The term “conventional cancer treatments” as used herein refers to any cancer drugs, biologics, or radiotherapy, or combination of cancer drugs and/or biologics and/or radiotherapy that have been tested and/or approved for therapeutic use in humans by the U.S. Food and Drug Administration, European Medicines Agency, or similar regulatory agency.

In another embodiment, the patient has been treated previously with an immune checkpoint inhibitor without a vinca alkaloid N-oxide. For example, the previous immune checkpoint therapy may be an anti-PD-1 therapy.

In another embodiment, the present disclosure provides therapeutic methods of treating a patient having cancer, the method comprising administering to the patient a therapeutically effective amount of a vinca alkaloid N-oxide and an immune checkpoint inhibitor, wherein the phenotypic status of the patient is overexpression of HIF. In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the present disclosure provides therapeutic methods of treating a patient having cancer, comprising administering to the patient therapeutically effective amounts of a vinca alkaloid N-oxide, an immune checkpoint inhibitor, and a third therapeutic agent.

In another embodiment, the present disclosure provides personalized medicine for cancer patients, and encompasses the selection of treatment options with the highest likelihood of successful outcome for individual cancer patients. In another aspect, the disclosure relates to the use of an assay(s) to predict the treatment outcome, e.g., the likelihood of favorable responses or treatment success, in patients having cancer.

In another embodiment, the present disclosure provides methods of selecting a patient, e.g., a human subject for treatment of cancer with a vinca alkaloid N-oxide and, optionally, an immune checkpoint inhibitor comprising obtaining a biological sample, e.g., blood cells, from the patient, testing a biological sample from the patient for the presence of a biomarker, e.g., overexpression of HIF, and selecting the patient for treatment if the biological sample contains that biomarker. In another embodiment, the methods further comprise administering a therapeutically effective amount of a vinca alkaloid N-oxide and, optionally, an immune checkpoint inhibitor, to the patient if the biological sample contains the biomarker. Examples of cancer biomarkers are provided in Table 1 and Table 2. In another embodiment, the cancer is a solid tumor. In another embodiment, the cancer is a hematological malignancy. In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the present disclosure provides methods of predicting treatment outcomes in a patient having cancer, comprising obtaining a biological sample, from the patient, testing the biological sample from the patient for the presence of a biomarker, e.g., overexpression of HIF, wherein the detection of the biomarker indicates the patient will respond favorably to administration of a therapeutically effective amount of a vinca alkaloid N-oxide and, optionally, an immune checkpoint inhibitor. Favorable responses include, but are not limited to, a decrease in tumor size and an increase in progression-free or overall survival.

In another embodiment, the present disclosure provides methods of treating cancer, comprising administering a therapeutically effective amount of a vinca alkaloid N-oxide and, optionally, an immune checkpoint inhibitor to a patient, e.g., a human subject, with cancer in whom the patient's cells contain a biomarker. In another embodiment, the patient is selected for treatment with a vinca alkaloid N-oxide and, optionally, an immune checkpoint inhibitor after the patient's cells have been determined to contain an overexpression of HIF.

In another embodiment, the method of treating a patient having cancer comprises obtaining a biological sample from the patient, determining whether the biological sample contains a biomarker, e.g., overexpression of HIF, and administering to the patient a therapeutically effective amount of a vinca alkaloid N-oxide and, optionally, an immune checkpoint inhibitor if the biological sample contains the biomarker. In another embodiment, the methods provided herein comprise determining whether the patient's cells contain an overexpression of HIF.

### I. Vinca Alkaloid N-Oxides

Vinca alkaloids are well-known chemotherapeutic agents originally isolated from the Madagascar periwinkle plant. Non-limiting exemplary vinca alkaloids include vinblastine, vincristine, vindesine, vinorelbine, and vinflunine.

The term “vinca alkaloid N-oxide” as used herein refers to a N_{b}-oxide or N_{b′}-oxide of a vinca alkaloid, and the pharmaceutically acceptable salts or solvates thereof. See Barnett et al., J. Med. Chem. 21:88-96 (1978) for discussion of the N_{b} and N_{b′} positions of the vinca alkaloid skeleton.

In one embodiment, the vinca alkaloid N-oxide is described in U.S. Pat. No. 8,048,872.

In another embodiment, the vinca alkaloid N-oxide is a vinca alkaloid N_{b}-oxide.

In another embodiment, the vinca alkaloid N-oxide is a vinca alkaloid N_{b′}-oxide, or a pharmaceutically acceptable salt or solvate thereof.

In another embodiment, the vinca alkaloid N_{b′}-oxide is represented by a compound having Formula I:

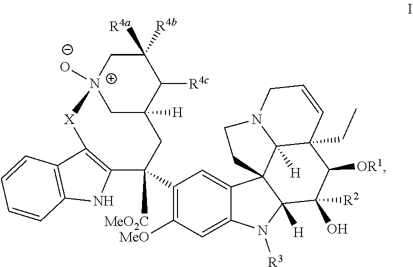

or a pharmaceutically acceptable salt or solvate thereof, wherein
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

In another embodiment, the vinca alkaloid N_{b′}-oxide is selected from the group consisting of vinblastine N_{b′}-oxide, vincristine N_{b′}-oxide, vindesine N_{b′}-oxide, vinorelbine N_{b′}-oxide, and vinflunine N_{b′}-oxide, and the pharmaceutically acceptable salts and solvates thereof.

In another embodiment, the vinca alkaloid N_{b′}-oxide is vinblastine N_{b′}-oxide, or a pharmaceutically acceptable salt or solvate thereof.

### II. Immune Checkpoint Inhibitors

Immune checkpoint inhibitors are therapies that blockade immune system inhibitor checkpoints. Immune checkpoints can be stimulatory or inhibitory. Blockade of inhibitory immune checkpoint activates immune system function and can be used for cancer immunotherapy. Pardoll, Nature Reviews. Cancer 12:252-64 (2012). Tumor cells turn off activated T cells when they attach to specific T-cell receptors. Immune checkpoint inhibitors prevent tumor cells from attaching to T cells, which results in T cells remaining activated. In effect, the coordinated action by cellular and soluble components combats pathogens and injuries by cancers. The modulation of immune system pathways may involve changing the expression or the functional activity of at least one component of the pathway to then modulate the response by the immune system. U.S. 2015/0250853. Examples of immune checkpoint inhibitors include PD-1 inhibitors, PD-L1 inhibitors, CTLA-4 inhibitors, LAG3 inhibitors, TIM3 inhibitors, cd47 inhibitors, VISTA inhibitors, TIGIT inhibitors, and B7-H1 inhibitors. Thus, in one embodiment, the immune checkpoint inhibitor is selected from the group consisting of a PD-1 inhibitor, a PD-L1 inhibitor, a CTLA-4 inhibitor, a LAG3 inhibitor, a TIM3 inhibitor, a VISTA inhibitor, a TIGIT inhibitor, and a cd47 inhibitor. In another embodiment, the immune checkpoint inhibitor is a CTLA-4 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{c}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

In another embodiment, the immune checkpoint inhibitor is a VISTA inhibitor. See Hernandez-Martinez et al., Journal of Thoracic Disease 10:6378-6382 (2018).

In another embodiment, the immune checkpoint inhibitor is a TIGIT inhibitor. T-cell immunoreceptor with immunoglobulin and ITIM domains (TIGIT) is an inhibitory receptor expressed on several immune cell types, including CD8⁺ T cells, natural killer, or NK, cells, T regulatory cells, or Tregs, and follicular T helper cells. TIGIT interacts with CD155 expressed on antigen-presenting cells or tumor cells to down-regulate T cell and natural killer (NK) cell functions. See, e.g., Harjunpaa, Clinical Experimental Immunology 200(2):108-19 (2020). TIGIT has been shown to be a mediator of resistance to existing checkpoint inhibitors, including anti-PD-1. TIGIT also directly suppresses the antitumor effector function on CD8 T cells. TIGIT inhibitors may include antibodies and small molecules. Non-limiting exemplary TIGIT inhibitor antibodies include vibostolimab (MK-7684), tiragolumab (RG6058), EOS_448, BMS-986207, BGB-A1217, MTIG7192A, AB154, ASP8374, and MK-7684.

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

In one embodiment, the PD-1 inhibitor is an anti-PD-1 antibody.

In one embodiment, the PD-L1 inhibitor is an anti-PD-L1 antibody.

In one embodiment, the CTLA-4 inhibitor is an anti-CTLA-4 antibody.

In one embodiment, the LAG3 inhibitor is an anti-LAG3 antibody.

In one embodiment, the TIM3 inhibitor is an anti-TIM3 antibody.

In one embodiment, the VISTA inhibitor is an anti-VISTA antibody.

In one embodiment, the TIGIT inhibitor is an anti-TIGIT antibody.

In one embodiment, the cd47 inhibitor is an anti-cd47 antibody.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053 and have the structure:

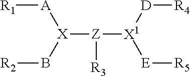

or a pharmaceutically acceptable salt thereof, wherein the compound comprises at least 5 amino acids useful as therapeutic agents capable of inhibiting the PD-1 signaling pathway.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

### III. Optional Therapeutic Agents

In certain therapeutic methods of the disclosure, a third therapeutic agent is administered to a cancer patient in combination with the vinca alkaloid N-oxide and the immune checkpoint inhibitor. The third therapeutic agent used in the therapeutic methods of the present disclosure are referred to as “optional therapeutic agents.” Such optional therapeutic agents useful in the treatment of cancer patients are known in the art.

Optional therapeutic agents are administered in an amount to provide their desired therapeutic effect. The effective dosage range for each optional therapeutic agent is known in the art, and the optional therapeutic agent is administered to an individual in need thereof within such established ranges.

A vinca alkaloid N-oxide, immune checkpoint inhibitor, and/or the optional therapeutic agent can be administered together as a single-unit dose or separately as multi-unit doses, and in any order, e.g., wherein a vinca alkaloid N-oxide is administered before the immune checkpoint inhibitor and/or the optional therapeutic agent, or vice versa. One or more doses of a vinca alkaloid N-oxide, the immune checkpoint inhibitor, and/or the optional therapeutic agent can be administered to the patient.

In one embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, the optional therapeutic agent is a chemotherapeutic agent or other anti-proliferative agent that can be administered in combination with a vinca alkaloid N-oxide to treat cancer. Examples of conventional therapies and anticancer agents that can be used in combination with a vinca alkaloid N-oxide include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Nonlimiting exemplary topoisomerase I inhibitors include topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include farnesyl transferase inhibitors, such as L-744832, DK8G557, tipifarnib, and lonafarnib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib.

Nonlimiting exemplary FMS-like tyrosine kinase inhibitors, which are compounds targeting, decreasing or inhibiting the activity of FMS-like tyrosine kinase receptors (Flt-3R) include interferon, I-β-D-arabinofuransylcytosine (ara-c), and bisulfan; and ALK inhibitors, which are compounds which target, decrease, or inhibit anaplastic lymphoma kinase.

Nonlimiting exemplary Flt-3 inhibitors include PKC412, midostaurin, a staurosporine derivative, SU11248, and MLN518.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SU1O1, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Ax1 receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C1-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a vinca alkaloid N-oxide, or a pharmaceutically acceptable salt thereof, include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors:NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, anticancer agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor. Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a cancer patient therapeutically effective amounts of a vinca alkaloid N-oxide and an immune checkpoint inhibitor and at least one additional anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, 5-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In some embodiments, methods provided herein comprise administering a vinca alkaloid N-oxide and an immune checkpoint inhibitor to a cancer patient in combination with radiation therapy. The methods provided herein are not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### IV. Therapeutic Methods

In the therapeutic methods provided herein, a vinca alkaloid N-oxide and an immune checkpoint inhibitor may be administered to a cancer patient under one or more of the following conditions: at different periodicities, at different durations, at different concentrations, by different administration routes, etc. An optional therapeutic, e.g., anticancer, agent may also be administered to the cancer patient.

In some embodiments, the vinca alkaloid N-oxide is administered prior to the immune checkpoint inhibitor, e.g., 0.5, 1, 2, 3, 4, 5, 10, 12, or 18 hours, 1, 2, 3, 4, 5, or 6 days, or 1, 2, 3, or 4 weeks prior to the administration of the immune checkpoint inhibitor.

In some embodiments, the vinca alkaloid N-oxide is administered after the immune checkpoint inhibitor, e.g., 0.5, 1, 2, 3, 4, 5, 10, 12, or 18 hours, 1, 2, 3, 4, 5, or 6 days, or 1, 2, 3, or 4 weeks after the administration of the immune checkpoint inhibitor.

In some embodiments, the vinca alkaloid N-oxide and the immune checkpoint inhibitor are administered concurrently but on different schedules, e.g., the vinca alkaloid N-oxide is administered daily while the immune checkpoint inhibitor is administered once a week, once every two weeks, once every three weeks, or once every four weeks. In other embodiments, the vinca alkaloid N-oxide is administered once a day while the immune checkpoint inhibitor is administered once a week, once every two weeks, once every three weeks, or once every four weeks.

The therapeutic methods provided herein comprise administering the vinca alkaloid N-oxide to a cancer patient in an amount which is effective to achieve its intended purpose. While individual needs vary, determination of optimal ranges of effective amounts of each component is within the skill of the art. Typically, the vinca alkaloid N-oxide may be administered in an amount from about 0.05 mg/kg to about 500 mg/kg, about 0.05 mg/kg to about 100 mg/kg, about 0.05 mg/kg to about 50 mg/kg, or about 0.05 mg/kg to about 10 mg/kg. The dosage of a composition can be at any dosage including, but not limited to, about 0.05 mg/week to about 25 mg/week. Particular doses include 0.05, 1, 2, 5, 10, 20, 500, and 100 mg/kg once weekly. In one embodiment, the vinca alkaloid N-oxide is administed once a week. These dosages are exemplary, but there can be individual instances in which higher or lower dosages are merited, and such are within the scope of this disclosure. In practice, the physician determines the actual dosing regimen that is most suitable for an individual patient, which can vary with the age, weight, and response of the particular patient.

The unit oral dose of the vinca alkaloid N-oxide may comprise from about 0.01 to about 1000 mg, e.g., about 0.01 to about 100 mg of the vinca alkaloid N-oxide. In one embodiment, the unit oral dose of the vinca alkaloid N-oxide is 0.05 mg, 1 mg, 3 mg, 5 mg, 7 mg, 9 mg, 10 mg 12 mg, 14 mg, 15 mg, 17 mg, 20 mg, 22 mg, 25 mg, 27 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. The unit dose may be administered one or more times daily, e.g., as one or more tablets or capsules. The unit does may also be administered by IV once a week. In practice, the physician determines the actual dosing regimen that is most suitable for an individual patient, which can vary with the age, weight, and response of the particular patient.

In addition to administering the vinca alkaloid N-oxide as a raw chemical, it may be administered as part of a pharmaceutical preparation or composition. In some embodiments, the pharmaceutical preparation or composition can include one or more pharmaceutically acceptable carriers, excipients, and/or auxiliaries. In some embodiments, the one or more carriers, excipients, and auxiliaries facilitate processing of the vinca alkaloid N-oxide into a preparation or composition which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the one or more carriers, excipients, and/or auxiliaries.

The pharmaceutical compositions of provided herein may be administered to any patient which may experience the beneficial effects of the vinca alkaloid N-oxide. Foremost among such patients are mammals, e.g., humans, although the methods and compositions provided herein are not intended to be so limited. Other patients include veterinary animals (cows, sheep, pigs, horses, dogs, cats and the like).

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers.

The present disclosure encompasses the use of solvates of the vinca alkaloid N-oxide. Solvates typically do not significantly alter the physiological activity or toxicity of a compound, and as such may function as pharmacological equivalents. The term “solvate” as used herein is a combination, physical association and/or solvation of a vinca alkaloid N-oxide with a solvent molecule such as, e.g., a disolvate, monosolvate or hemisolvate, where the ratio of solvent molecule to vinca alkaloid N-oxide is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. The vinca alkaloid N-oxide can be present as solvated forms with a pharmaceutically acceptable solvent, such as water, methanol, ethanol, and the like, and it is intended that the disclosure includes both solvated and unsolvated forms of the vinca alkaloid N-oxide. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate involves dissolving a vinca alkaloid N-oxide in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

Therapeutically effective amounts of the vinca alkaloid N-oxide and the immune checkpoint inhibitor formulated in accordance with standard pharmaceutical practices, are administered to a human patient in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

The vinca alkaloid N-oxide and the immune checkpoint inhibitor can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein the vinca alkaloid N-oxide and the immune checkpoint inhibitor are administered in an effective amount to achieve its intended purpose. The exact formulation, route of administration, and dosage is determined by an individual physician in view of the diagnosed condition or disease. Dosage amount and interval can be adjusted individually to provide levels of the vinca alkaloid N-oxide and the immune checkpoint inhibitor that is sufficient to maintain therapeutic effects.

Toxicity and therapeutic efficacy of the vinca alkaloid N-oxide and the immune checkpoint inhibitor can be determined by standard pharmaceutical procedures in cell cultures or experimental animals, e.g., for determining the maximum tolerated dose (MTD) of a compound, which defines as the highest dose that causes no toxicity in a patient. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of the vinca alkaloid N-oxide and the immune checkpoint inhibitor required for use in therapy varies with the nature of the condition being treated, the length of time that activity is desired, and the age and the condition of the patient, and ultimately is determined by the attendant physician. For example, dosage amounts and intervals can be adjusted individually to provide plasma levels of the vinca alkaloid N-oxide and immune checkpoint inhibitor that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, the vinca alkaloid N-oxide and immune checkpoint inhibitor can be administered at a frequency of: one dose per day; four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

The immune checkpoint inhibitor is administered in therapeutically effective amounts. When the immune checkpoint inhibitor is a monoclonal antibody, 1-20 mg/kg is administered as an intravenous infusion every 2-4 weeks. For example, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg and 2000 mg of the antibody may be administered.

For example, when the immune checkpoint inhibitor is the anti-PD-1 antibody nivolumab, 3 mg/kg may be administered by intravenous infusion over 60 minutes every two weeks. When the immune checkpoint inhibitor is the anti-PD-1 antibody pembrolizumab, 2 mg/kg may be administered by intravenous infusion over 30 minutes every two or three weeks. When the immune checkpoint inhibitor is the anti-PD-L1 antibody avelumab, 10 mg/kg may be administered by intravenous infusion as frequently as every 2 weeks. Disis et al., J. Clin Oncol. 33 (2015) (suppl; abstr 5509). When the immune checkpoint inhibitor is the anti-PD-L1 antibody MPDL3280A, 20 mg/kg may be administered by intravenous infusion every 3 weeks. Herbst et al., Nature 515:563-80 (2014). When the immune checkpoint inhibitor is the anti-CTLA-4 antibody ipilumumab, 3 mg/kg may be administered by intravenous infusion over 90 minutes every 3 weeks. When the immune checkpoint inhibitor is the anti-CTLA-4 antibody tremelimumab, 15 mg/kg may be administered by intravenous infusion every 12 weeks. Naido et al., British Journal of Cancer 111:2214-19 (2014); Drugs RD, 10:123-32 (2010). When the immune checkpoint inhibitor is the anti-LAG3 antibody GSK2831781, 1.5 to 5 mg/kg may be administered by intravenous infusion over 120 minutes every 2-4 weeks. When the immune checkpoint inhibitor is an anti-TIM3 antibody, 1-5 mg/kg may be administered by intravenous infusion over 30-90 minutes every 2-4 weeks. When an inhibitor of indoleamine 2,3-dioxygenase (IDO) pathway is inhibitor indoximod in combination with temozolomide, 18.5 mg/kg/dose BID with an escalation to 27.7 mg/kg/dose BID of indoximod with 200 mg/m² every 5 days of temozolomide.

In one embodiment, the immune checkpoint inhibitor is an antibody and 1-20 mg/kg is administered by intravenous infusion every 2-4 weeks. In another embodiment, 50-2000 mg of the antibody is administered by intravenous infusion every 2-4 weeks. In another embodiment, the vinca alkaloid N-oxide is administered prior to administration of the antibody. In another embodiment, the vinca alkaloid N-oxide is administered 3-7 days prior to the day of administration of the antibody. In another embodiment, the vinca alkaloid N-oxide is also administered the day the antibody is administered and on consecutive days thereafter until disease progression or until the vinca alkaloid N-oxide administration is no longer beneficial.

In one embodiment, the cancer patient receives 2 mg/kg pembrolizumab administered by intravenous infusion every three weeks and about 0.1 to 100 mg of the vinca alkaloid N-oxide administered for 1-7 days prior to pembrolizumab administration, optionally, on the day of pembrolizumab administration, and, optionally, thereafter until disease progression or until there is no therapeutic benefit. In another embodiment, the cancer patient has tumors with a biomarker, e.g., overexpression of HIF.

In another embodiment, the cancer patient receives 3 mg/kg nivolumab administered by intravenous infusion every 2 weeks and about 0.1 to 100 mg of the vinca alkaloid N-oxide administered for 1-7 days prior to nivolumab administration, optionally, on the day of nivolumab administration, and, optionally, thereafter until disease progression or until there is no therapeutic benefit. In another embodiment, the cancer patient has tumors with a biomarker, e.g., overexpression of HIF.

In another embodiment, the cancer patient receives 3 mg/kg nivolumab administered by intravenous infusion every 2 weeks and about 0.1 to 100 mg of the vinca alkaloid N-oxide administered for 1-7 days prior to nivolumab administration, optionally, on the day of nivolumab administration, and, optionally, thereafter until disease progression or until there is no therapeutic benefit. In another embodiment, the cancer patient has tumors with a biomarker, e.g., overexpression of HIF.

Representative dosing regimens for certain immune checkpoint inhibitors to treat certain cancers are provided in Table 6.

TABLE 6

| | Body-Weight- | | | | |
|---|---|---|---|---|---|
| Drug | Based Dose | | Flat Dose | | Clinical Applications |
| Ipilimumab | 3 | mg/kg Q3W | | | Metastatic melanoma |
| | 10 | mg/kg Q3W | | | Cutaneous melanoma |
| | | | | | Advanced renal cell carcinoma |
| Nivolumab | 3 | mg/kg Q2W | 240 | mg Q2W | Metastatic melanoma |
| | | | 480 | mg Q4W | Metastatic NSCLC |
| | | | | | Hodgkin lymphoma |
| | | | | | Advanced renal cell carcinoma |
| | | | | | Advanced or metastatic |
| | | | | | urothelial carcinoma |
| | | | | | Metastatic colorectal cancer |
| | | | | | Hepatocellular carcinoma |
| Pembrolizumab | 2 | mg/kg Q3W | 200 | mg Q3W | Melanoma |
| | | | 400 | mg Q6W | NSCLC |
| | | | | | Head and neck squamous cell |
| | | | | | cancer |
| | | | | | Classical Hodgkin lymphoma |
| | | | | | Primary mediastinal large b-cell |
| | | | | | lymphoma |
| | | | | | Urothelial carcinoma |
| | | | | | Microsatellite instability-high |
| | | | | | cancer |
| | | | | | Gastric cancer |
| | | | | | Cervical cancer |
| | | | | | Hepatocellular carcinoma |
| | | | | | Merkel cell carcinoma |
| Cemiplimab | | | 350 | mg Q3W | Metastatic CSCC |
| | | | | | Locally advanced CSCC |
| Atezolizumab | | | 840 | mg Q2W | Urothelical Carcinoma |
| | | | 1200 | mg Q3W | NSCLC |
| | | | 1680 | mg Q4W | TNBC |
| | | | | | Metastatic treatment of TNBC |
| Avelumab | 10 | mg/kg Q2W | 800 | mg Q2W | Metastatic Merkel cell |
| | | | | | carcinoma |
| | | | | | Advanced or metastatic |
| | | | | | urothelial carcinoma |
| | | | | | Advanced renal cell carcinoma |
| | | | | | (+axitinib) |
| Durvalumab | 10 | mg/kg Q2W | 750 | mg Q2W | Locally advanced or metastatic |
| | | | 1500 | mg Q4W | urothelical carcinoma |
| | | | | | Unresectable stage III NSCLC |

In one embodiment, the one or more optional immune checkpoint inhibitors is an antibody, and 1-20 mg/kg is administered to the subject by intravenous infusion every 2-4 weeks. In another embodiment, 20-2000 mg of the antibody is administered to the subject by intravenous infusion every 2-4 weeks. In another embodiment, the vinca alkaloid N-oxide is administered prior to administration of the antibody. In another embodiment, the vinca alkaloid N-oxide is administered to the subject 1, 2, 3, 4, 5, 6, or 7 days prior to the day of administration of the antibody. In another embodiment, the vinca alkaloid N-oxide is administered to the subject the day the antibody is administered. In another embodiment, the vinca alkaloid N-oxide is administered to the subject 1, 2, 3, 4, 5, 6, or 7 days after the day of administration of the antibody.

For example, the subject receives pembrolizumab administered by intravenous infusion every three weeks and vinblastine N_{b′}-oxide adminstered three times a week by intravenous or two times a week by subcutaneous infusion, wherein the first dose of vinblastine N_{b′}-oxide is administered prior to the first dose of pembrolizumab, the first dose of vinblastine N_{b′}-oxide is administered on the same day as the first dose of pembrolizumab, or the first dose of vinblastine N_{b′}-oxide is administered after to the first dose of pembrolizumab, e.g., until disease progression or until there is no therapeutic benefit.

For example, the subject receives nivolumab administered by intravenous infusion every two weeks and vinblastine N_{b′}-oxide adminstered three times a week by intravenous or two times a week by subcutaneous infusion, wherein the first dose of vinblastine N_{b′}-oxide is administered prior to the first dose of nivolumab, the first dose of vinblastine N_{b′}-oxide is administered on the same day as the first dose of nivolumab, or the first dose vinblastine N_{b′}-oxide is administered after to the first dose of nivolumab, e.g., until disease progression or until there is no therapeutic benefit.

In another embodiment, the treatment of the cancer patient with a vinca alkaloid N-oxide and an immune checkpoint inhibitor induces anti-proliferative response faster than when the immune checkpoint inhibitor is administered alone.

### V. Biomarkers

The term “biomarker” as used herein refers to any biological compound, such as a gene, a protein, a fragment of a protein, a peptide, a polypeptide, a nucleic acid, etc., that can be detected and/or quantified in a cancer patient in vivo or in a biological sample obtained from a cancer patient. A biomarker can be the entire intact molecule, or it can be a portion or fragment thereof. In one embodiment, the expression level of the biomarker is measured. The expression level of the biomarker can be measured, for example, by detecting the protein or RNA, e.g., mRNA, level of the biomarker. In some embodiments, portions or fragments of biomarkers can be detected or measured, for example, by an antibody or other specific binding agent. In some embodiments, a measurable aspect of the biomarker is associated with a given state of the patient, such as a particular stage of cancer. For biomarkers that are detected at the protein or RNA level, such measurable aspects may include, for example, the presence, absence, or concentration, i.e., expression level, of the biomarker in a cancer patient, or biological sample obtained from the cancer patient. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

Biomarkers include, but are not limited, the genes listed in Table 1 and/or Table 2. See, e.g., Le and Courter, Cancer Metastasis Rev. 27:351-362 (2008). In one embodiment, the measurable aspect of the biomarker is its expression status. In one embodiment, the measurable aspect of the biomarker is its mutation status.

TABLE 1

| Gene | Gene synonym | Gene description |
|---|---|---|
| A2M | CPAMD5, FWP007, S863-7 | Alpha-2-macroglobulin |
| ABCB1 | ABC20, CD243, CLCS, GP170, | ATP-binding cassette, sub-family B |
| | MDR1, P-gp, PGY1 | (MDR/TAP), member 1 |
| ABCC1 | GS-X, MRP, MRP1 | ATP-binding cassette, sub-family C |
| | | (CFTR/MRP), member 1 |
| ABCC2 | CMOAT, cMRP, DJS, MRP2 | ATP-binding cassette, sub-family C |
| | | (CFTR/MRP), member 2 |
| ABCC3 | cMOAT2, EST90757, MLP2, | ATP-binding cassette, sub-family C |
| | MOAT-D, MRP3 | (CFTR/MRP), member 3 |
| ABCC5 | EST277145, MOAT-C, MRP5, | ATP-binding cassette, sub-family C |
| | SMRP | (CFTR/MRP), member 5 |
| ABCC6 | ARA, EST349056, MLP1, | ATP-binding cassette, sub-family C |
| | MRP6, PXE, URG7 | (CFTR/MRP), member 6 |
| ABCG2 | ABCP, BCRP, CD338, | ATP-binding cassette, sub-family G |
| | EST157481, MXR | (WHITE), member 2 (Junior blood group) |
| ABL1 | ABL, c-ABL, JTK7, p150 | ABL proto-oncogene 1, non-receptor |
| | | tyrosine kinase |
| ABL2 | ABLL, ARG | ABL proto-oncogene 2, non-receptor |
| | | tyrosine kinase |
| ACAP1 | CENTB1, KIAA0050 | ArfGAP with coiled-coil, ankyrin repeat |
| | | and PH domains 1 |
| ACLY | ACL, ATPCL, CLATP | ATP citrate lyase |
| ACPP | ACP-3, ACP3 | Acid phosphatase, prostate |
| ACVR1B | ActRIB, ACVRLK4, ALK4, | Activin A receptor, type IB |
| | SKR2 | |
| ACVR2A | ACTRII, ACVR2 | Activin A receptor, type IIA |
| ACVR2B | ActR-IIB | Activin A receptor, type IIB |
| ADAM9 | CORD9, KIAA0021, MCMP, | ADAM metallopeptidase domain 9 |
| | MDC9, Mltng | |
| ADAMTS1 | C3-C5, KIAA1346, METH1 | ADAM metallopeptidase with |
| | | thrombospondin type 1 motif, 1 |
| ADAMTS14 | | ADAM metallopeptidase with |
| | | thrombospondin type 1 motif, 14 |
| ADAMTS18 | ADAMTS21 | ADAM metallopeptidase with |
| | | thrombospondin type 1 motif, 18 |
| ADAMTS20 | GON-1 | ADAM metallopeptidase with |
| | | thrombospondin type 1 motif, 20 |
| ADAMTS3 | ADAMTS-4, KIAA0366 | ADAM metallopeptidase with |
| | | thrombospondin type 1 motif, 3 |
| ADAMTS4 | ADAMTS-2, ADMP-1, | ADAM metallopeptidase with |
| | KIAA0688 | thrombospondin type 1 motif, 4 |
| ADAMTS5 | ADAMTS11, ADMP-2 | ADAM metallopeptidase with |
| | | thrombospondin type 1 motif, 5 |
| ADAMTS6 | ADAM-TS6 | ADAM metallopeptidase with |
| | | thrombospondin type 1 motif, 6 |
| ADAMTS8 | ADAM-TS8, FLJ41712, METH2 | ADAM metallopeptidase with |
| | | thrombospondin type 1 motif, 8 |
| ADAMTS9 | KIAA1312 | ADAM metallopeptidase with |
| | | thrombospondin type 1 motif, 9 |
| ADM | AM | Adrenomedullin |
| ADRA1B | | Adrenoceptor alpha 1B |
| AFP | FETA, HPAFP | Alpha-fetoprotein |
| AGER | RAGE | Advanced glycosylation end product- |
| | | specific receptor |
| AHR | bHLHe76 | Aryl hydrocarbon receptor |
| AHSG | A2HS, FETUA, HSGA | Alpha-2-HS-glycoprotein |
| AKAP12 | AKAP250, SSeCKS | A kinase (PRKA) anchor protein 12 |
| AKR1B1 | ALDR1, AR | Aldo-keto reductase family 1, member B1 |
| | | (aldose reductase) |
| AKT1 | AKT, PKB, PRKBA, RAC | V-akt murine thymoma viral oncogene |
| | | homolog 1 |
| AKT2 | | V-akt murine thymoma viral oncogene |
| | | homolog 2 |
| AKT3 | PKBG, PRKBG, RAC-gamma | V-akt murine thymoma viral oncogene |
| | | homolog 3 |
| ALB | | Albumin |
| ALCAM | CD166, MEMD | Activated leukocyte cell adhesion molecule |
| ALDOA | | Aldolase A, fructose-bisphosphate |
| ALDOB | | Aldolase B, fructose-bisphosphate |
| ALDOC | | Aldolase C, fructose-bisphosphate |
| ALPL | HOPS, TNSALP | Alkaline phosphatase, liver/bone/kidney |
| ALPP | | Alkaline phosphatase, placental |
| ANG | RNASE5 | Angiogenin, ribonuclease, RNase A family, 5 |
| ANGPT1 | Ang1, KIAA0003 | Angiopoietin 1 |
| ANGPT2 | Ang2 | Angiopoietin 2 |
| ANXA1 | ANX1, LPC1 | Annexin A1 |
| ANXA11 | ANX11 | Annexin A11 |
| ANXA2 | ANX2, ANX2L4, CAL1H, LIP2, | Annexin A2 |
| | LPC2D | |
| ANXA4 | ANX4 | Annexin A4 |
| ANXA7 | ANX7 | Annexin A7 |
| AOC3 | HPAO, VAP-1, VAP1 | Amine oxidase, copper containing 3 |
| AP2B1 | ADTB2, CLAPB1 | Adaptor-related protein complex 2, beta 1 |
| | | subunit |
| APAF1 | APAF-1, CED4 | Apoptotic peptidase activating factor 1 |
| APEX1 | APE, APE-1, APEN, APEX, | APEX nuclease (multifunctional DNA |
| | APX, HAP1, REF-1, REF1 | repair enzyme) 1 |
| APOA1 | | Apolipoprotein A-I |
| APOA2 | | Apolipoprotein A-II |
| APOC1 | | Apolipoprotein C-I |
| APOC3 | | Apolipoprotein C-III |
| APOD | | Apolipoprotein D |
| APOE | AD2 | Apolipoprotein E |
| APPBP2 | Hs.84084, KIAA0228, PAT1 | Amyloid beta precursor protein |
| | | (cytoplasmic tail) binding protein 2 |
| AR | AIS, DHTR, HUMARA, | Androgen receptor |
| | NR3C4, SBMA, SMAX1 | |
| AREG | AREGB, SDGF | Amphiregulin |
| ARG2 | | Arginase 2 |
| ARNT | bHLHe2, HIF-1beta | Aryl hydrocarbon receptor nuclear |
| | | translocator |
| ASPH | BAH, CASQ2BP1, HAAH, | Aspartate beta-hydroxylase |
| | JCTN | |
| ATM | ATA, ATC, ATD, ATDC, TEL1, | ATM serine/threonine kinase |
| | TELO1 | |
| ATOH1 | bHLHa14, HATH1, MATH-1, | Atonal homolog 1 (Drosophila) |
| | Math1 | |
| ATP7B | WND | ATPase, Cu++ transporting, beta |
| | | polypeptide |
| AURKA | AIK, ARK1, AurA, BTAK, | Aurora kinase A |
| | PPP1R47, STK15, STK6, STK7 | |
| AURKB | Aik2, AIM-1, ARK2, AurB, | Aurora kinase B |
| | IPL1, PPP1R48, STK12, STK5 | |
| AZGP1 | ZA2G, ZAG | Alpha-2-glycoprotein 1, zinc-binding |
| B2M | | Beta-2-microglobulin |
| BAD | BBC2, BCL2L8 | BCL2-associated agonist of cell death |
| BAG1 | | BCL2-associated athanogene |
| BAI1 | | Brain-specific angiogenesis inhibitor 1 |
| BAX | BCL2L4 | BCL2-associated X protein |
| BCL11A | BCL11A-L, BCL11A-S, | B-cell CLL/lymphoma 11A (zinc finger |
| | BCL11A-XL, CTIP1, EVI9, | protein) |
| | HBFQTL5, ZNF856 | |
| BCL2 | Bcl-2, PPP1R50 | B-cell CLL/lymphoma 2 |
| BCL2A1 | ACC-1, ACC-2, BCL2L5, BFL1, | BCL2-related protein A1 |
| | GRS, HBPA1 | |
| BCL2L1 | Bcl-X, bcl-xL, bcl-xS, BCL2L, | BCL2-like 1 |
| | BCLX, PPP1R52 | |
| BCL2L2 | BCL-W, KIAA0271, PPP1R51 | BCL2-like 2 |
| BCL2L2- | | BCL2L2-PABPN1 readthrough |
| PABPN1 | | |
| BCL3 | BCL4, D19S37 | B-cell CLL/lymphoma 3 |
| BCL6 | BCL5, BCL6A, LAZ3, ZBTB27, | B-cell CLL/lymphoma 6 |
| | ZNF51 | |
| BDNF | | Brain-derived neurotrophic factor |
| BIRC2 | API1, c-IAP1, cIAP1, hiap-2, | Baculoviral IAP repeat containing 2 |
| | MIHB, RNF48 | |
| BIRC3 | API2, c-IAP2, cIAP2, hiap-1, | Baculoviral IAP repeat containing 3 |
| | MALT2, MIHC, RNF49 | |
| BIRC5 | API4, EPR-1, survivin | Baculoviral IAP repeat containing 5 |
| BIRC6 | BRUCE | Baculoviral IAP repeat containing 6 |
| BLK | MGC10442 | BLK proto-oncogene, Src family tyrosine |
| | | kinase |
| BLMH | BH | Bleomycin hydrolase |
| BMI1 | PCGF4, RNF51 | BMI1 proto-oncogene, polycomb ring |
| | | finger |
| BMP2 | BMP2A | Bone morphogenetic protein 2 |
| BMP4 | BMP2B | Bone morphogenetic protein 4 |
| BNIP3 | Nip3 | BCL2/adenovirus E1B 19 kDa interacting |
| | | protein 3 |
| BNIP3L | BNIP3a, Nix | BCL2/adenovirus E1B 19 kDa interacting |
| | | protein 3-like |
| BRCA1 | BRCC1, PPP1R53, RNF53 | Breast cancer 1, early onset |
| BRCA2 | BRCC2, FACD, FAD, FAD1, | Breast cancer 2, early onset |
| | FANCD, FANCD1 | |
| BRMS1 | DKFZP564A063 | Breast cancer metastasis suppressor 1 |
| BTG2 | MGC126063, MGC126064, | BTG family, member 2 |
| | PC3, TIS21 | |
| C18orf8 | HsT2591, MIC-1, MIC1 | Chromosome 18 open reading frame 8 |
| C1QBP | gC1Q-R, gC1qR, HABP1, p32, | Complement component 1, q |
| | SF2p32 | subcomponent binding protein |
| C6 | | Complement component 6 |
| C7 | | Complement component 7 |
| CA8 | CALS, CARP | Carbonic anhydrase VIII |
| CALCA | CALC1 | Calcitonin-related polypeptide alpha |
| CALM1 | CALML2, CAMI, DD132, | Calmodulin 1 (phosphorylase kinase, delta) |
| | PHKD | |
| CALM2 | CAMII, PHKD | Calmodulin 2 (phosphorylase kinase, delta) |
| CALM3 | PHKD | Calmodulin 3 (phosphorylase kinase, delta) |
| CALR | cC1qR, CRT, FLJ26680, RO, | Calreticulin |
| | SSA | |
| CANX | CNX, IP90, P90 | Calnexin |
| CAPN6 | CalpM, CANPX, CAPNX | Calpain 6 |
| CASC3 | BTZ, MLN51 | Cancer susceptibility candidate 3 |
| CASP1 | ICE, IL1BC | Caspase 1, apoptosis-related cysteine |
| | | peptidase |
| CASP10 | MCH4 | Caspase 10, apoptosis-related cysteine |
| | | peptidase |
| CASP2 | ICH1, MGC2181, NEDD2, | Caspase 2, apoptosis-related cysteine |
| | PPP1R57 | peptidase |
| CASP3 | apopain, CPP32, CPP32B, Yama | Caspase 3, apoptosis-related cysteine |
| | | peptidase |
| CASP4 | ICE(rel)II, ICH-2, TX | Caspase 4, apoptosis-related cysteine |
| | | peptidase |
| CASP5 | ICE(rel)III | Caspase 5, apoptosis-related cysteine |
| | | peptidase |
| CASP6 | MCH2 | Caspase 6, apoptosis-related cysteine |
| | | peptidase |
| CASP7 | CMH-1, ICE-LAP3, MCH3 | Caspase 7, apoptosis-related cysteine |
| | | peptidase |
| CASP8 | Casp-8, FLICE, MACH, MCH5 | Caspase 8, apoptosis-related cysteine |
| | | peptidase |
| CASP9 | APAF-3, ICE-LAP6, MCH6, | Caspase 9, apoptosis-related cysteine |
| | PPP1R56 | peptidase |
| CAT | | Catalase |
| CAV1 | CAV | Caveolin 1, caveolae protein, 22 kDa |
| CBL | c-Cbl, CBL2, RNF55 | Cbl proto-oncogene, E3 ubiquitin protein |
| | | ligase |
| CCKBR | | Cholecystokinin B receptor |
| CCL11 | eotaxin, MGC22554, SCYA11 | Chemokine (C-C motif) ligand 11 |
| CCL13 | CKb10, MCP-4, MGC17134, | Chemokine (C-C motif) ligand 13 |
| | NCC-1, SCYA13, SCYL1 | |
| CCL14 | CKb1, HCC-1, HCC-3, MCIF, | Chemokine (C-C motif) ligand 14 |
| | NCC-2, SCYA14, SCYL2 | |
| CCL16 | CKb12, HCC-4, LCC-1, LEC, | Chemokine (C-C motif) ligand 16 |
| | LMC, Mtn-1, NCC-4, SCYA16, | |
| | SCYL4 | |
| CCL18 | AMAC-1, CKb7, DC-CK1, | Chemokine (C-C motif) ligand 18 |
| | DCCK1, MIP-4, PARC, | (pulmonary and activation-regulated) |
| | SCYA18 | |
| CCL19 | CKb11, ELC, exodus-3, MIP-3b, | Chemokine (C-C motif) ligand 19 |
| | SCYA19 | |
| CCL2 | GDCF-2, HC11, MCAF, MCP-1, | Chemokine (C-C motif) ligand 2 |
| | MCP1, MGC9434, SCYA2, | |
| | SMC-CF | |
| CCL21 | 6Ckine, CKb9, ECL, exodus-2, | Chemokine (C-C motif) ligand 21 |
| | SCYA21, SLC, TCA4 | |
| CCL23 | Ckb-8, CKb8, MIP-3, MPIF-1, | Chemokine (C-C motif) ligand 23 |
| | SCYA23 | |
| CCL3 | G0S19-1, LD78ALPHA, MIP-1- | Chemokine (C-C motif) ligand 3 |
| | alpha, SCYA3 | |
| CCL4 | Act-2, AT744.1, LAG1, MIP-1- | Chemokine (C-C motif) ligand 4 |
| | beta, SCYA4 | |
| CCL5 | D17S136E, MGC17164, | Chemokine (C-C motif) ligand 5 |
| | RANTES, SCYA5, SISd, | |
| | TCP228 | |
| CCL7 | FIC, MARC, MCP-3, MCP3, | Chemokine (C-C motif) ligand 7 |
| | NC28, SCYA6, SCYA7 | |
| CCL8 | HC14, MCP-2, SCYA8 | Chemokine (C-C motif) ligand 8 |
| CCNA1 | CT146 | Cyclin A1 |
| CCNA2 | CCN1, CCNA | Cyclin A2 |
| CCNB1 | CCNB | Cyclin B1 |
| CCNB2 | HsT17299 | Cyclin B2 |
| CCND1 | BCL1, D11S287E, PRAD1, | Cyclin D1 |
| | U21B31 | |
| CCND2 | | Cyclin D2 |
| CCNE1 | CCNE | Cyclin E1 |
| CCNE2 | CYCE2 | Cyclin E2 |
| CCNG1 | CCNG | Cyclin G1 |
| CCNG2 | | Cyclin G2 |
| CCNH | CycH, p34, p37 | Cyclin H |
| CCR10 | GPR2 | Chemokine (C-C motif) receptor 10 |
| CCR7 | BLR2, CD197, CDw197, | Chemokine (C-C motif) receptor 7 |
| | CMKBR7, EBI1 | |
| CD14 | | CD14 molecule |
| CD27 | S152, TNFRSF7, Tp55 | CD27 molecule |
| CD36 | FAT, GP3B, GP4, GPIV, | CD36 molecule (thrombospondin receptor) |
| | SCARB3 | |
| CD38 | | CD38 molecule |
| CD40 | Bp50, p50, TNFRSF5 | CD40 molecule, TNF receptor superfamily |
| | | member 5 |
| CD40LG | CD154, CD40L, gp39, hCD40L, | CD40 ligand |
| | HIGM1, IMD3, TNFSF5, TRAP | |
| CD44 | CD44R, CSPG8, HCELL, IN, | CD44 molecule (Indian blood group) |
| | MC56, MDU2, MDU3, MIC4, | |
| | Pgp1 | |
| CD46 | MCP, MGC26544, MIC10, | CD46 molecule, complement regulatory |
| | TLX, TRA2.10 | protein |
| CD52 | CDW52 | CD52 molecule |
| CD59 | 16.3A5, EJ16, EJ30, EL32, | CD59 molecule, complement regulatory |
| | G344, MIC11, MIN1, MIN2, | protein |
| | MIN3, MSK21, p18-20 | |
| CD70 | CD27L, CD27LG, TNFSF7 | CD70 molecule |
| CD74 | DHLAG | CD74 molecule, major histocompatibility |
| | | complex, class II invariant chain |
| CD82 | IA4, KAI1, R2, ST6, TSPAN27 | CD82 molecule |
| CD9 | BA2, MIC3, MRP-1, P24, | CD9 molecule |
| | TSPAN29 | |
| CDC16 | ANAPC6, APC6, CUT9 | Cell division cycle 16 |
| CDC20 | CDC20A, p55CDC | Cell division cycle 20 |
| CDC25A | | Cell division cycle 25A |
| CDC25B | | Cell division cycle 25B |
| CDC25C | CDC25, PPP1R60 | Cell division cycle 25C |
| CDC34 | E2-CDC34, UBC3, UBE2R1 | Cell division cycle 34 |
| CDC37 | P50CDC37 | Cell division cycle 37 |
| CDC6 | CDC18L | Cell division cycle 6 |
| CDH1 | CD324, UVO, uvomorulin | Cadherin 1, type 1, E-cadherin (epithelial) |
| CDH17 | cadherin, HPT-1 | Cadherin 17, LI cadherin (liver-intestine) |
| CDH5 | 7B4, CD144 | Cadherin 5, type 2 (vascular endothelium) |
| CDK1 | CDC2, CDC28A | Cyclin-dependent kinase 1 |
| CDK2 | | Cyclin-dependent kinase 2 |
| CDK4 | PSK-J3 | Cyclin-dependent kinase 4 |
| CDK6 | PLSTIRE | Cyclin-dependent kinase 6 |
| CDK7 | CAK, CAK1, CDKN7, MO15, | Cyclin-dependent kinase 7 |
| | STK1 | |
| CDKN1A | CAP20, CDKN1, CIP1, P21, | Cyclin-dependent kinase inhibitor 1A (p21, |
| | p21CIP1, p21Cip1/Waf1, SDI1, | Cip1) |
| | WAF1 | |
| CDKN1C | BWCR, BWS, KIP2, P57 | Cyclin-dependent kinase inhibitor 1C (p57, |
| | | Kip2) |
| CDKN2A | ARF, CDK4I, CDKN2, CMM2, | Cyclin-dependent kinase inhibitor 2A |
| | INK4, INK4a, MLM, MTS1, | |
| | p14, p14ARF, p16, p16INK4a, | |
| | p19, p19Arf | |
| CEACAM5 | CD66e, CEA | Carcinoembryonic antigen-related cell |
| | | adhesion molecule 5 |
| CEACAM6 | CD66c, NCA | Carcinoembryonic antigen-related cell |
| | | adhesion molecule 6 (non-specific cross |
| | | reacting antigen) |
| CENPF | hcp-1 | Centromere protein F, 350/400 kDa |
| CFHR1 | CFHL, CFHL1, CFHL1P, | Complement factor H-related 1 |
| | CFHR1P, FHR1, H36-1, H36-2, | |
| | HFL1, HFL2 | |
| CFLAR | c-FLIP, CASH, CASP8AP1, | CASP8 and FADD-like apoptosis regulator |
| | Casper, CLARP, FLAME, FLIP, | |
| | I-FLICE, MRIT | |
| CFTR | ABC35, ABCC7, CF, | Cystic fibrosis transmembrane conductance |
| | CFTR/MRP, dJ760C5.1, MRP7, | regulator (ATP-binding cassette sub-family |
| | TNR-CFTR | C, member 7) |
| CGA | FSHA, GPHa, GPHA1, HCG, | Glycoprotein hormones, alpha polypeptide |
| | LHA, TSHA | |
| CGB | CGB3 | Chorionic gonadotropin, beta polypeptide |
| CGB5 | HCG | Chorionic gonadotropin, beta polypeptide 5 |
| CGB7 | CG-beta-a | Chorionic gonadotropin, beta polypeptide 7 |
| CGB8 | | Chorionic gonadotropin, beta polypeptide 8 |
| CHD7 | CRG, FLJ20357, FLJ20361, | Chromodomain helicase DNA binding |
| | KIAA1416 | protein 7 |
| CHEK1 | CHK1 | Checkpoint kinase 1 |
| CHEK2 | bA444G7, CDS1, CHK2, | Checkpoint kinase 2 |
| | HuCds1, PP1425, RAD53 | |
| CHFR | FLJ10796, RNF196 | Checkpoint with forkhead and ring finger |
| | | domains, E3 ubiquitin protein ligase |
| CHGA | | Chromogranin A (parathyroid secretory |
| | | protein 1) |
| CHI3L1 | GP39, YKL40 | Chitinase 3-like 1 (cartilage glycoprotein-39) |
| CHP2 | | Calcineurin-like EF-hand protein 2 |
| CIB2 | DFNB48, KIP2, USH1J | Calcium and integrin binding family |
| | | member 2 |
| CKB | CKBB | Creatine kinase, brain |
| CKS1B | CKS1, ckshs1 | CDC28 protein kinase regulatory subunit 1B |
| CKS2 | | CDC28 protein kinase regulatory subunit 2 |
| CLDN3 | C7orf1, CPE-R2, CPETR2, | Claudin 3 |
| | HRVP1, RVP1 | |
| CLDN4 | CPE-R, CPETR, CPETR1, | Claudin 4 |
| | hCPE-R, WBSCR8 | |
| CLDN7 | CEPTRL2, CPETRL2, Hs.84359 | Claudin 7 |
| CLEC3B | TN, TNA | C-type lectin domain family 3, member B |
| CLIC1 | NCC27, p64CLCP | Chloride intracellular channel 1 |
| CLIP1 | CLIP, CLIP-170, CLIP170, | CAP-GLY domain containing linker |
| | CYLN1, RSN | protein 1 |
| CLSTN1 | CDHR12, CSTN1, KIAA0911 | Calsyntenin 1 |
| CLU | APOJ, CLI, CLU1, CLU2, | Clusterin |
| | KUB1, SGP-2, SP-40, TRPM-2 | |
| CNN1 | Sm-Calp, SMCC | Calponin 1, basic, smooth muscle |
| CNTF | HCNTF | Ciliary neurotrophic factor |
| COL11A1 | CO11A1, COLL6, STL2 | Collagen, type XI, alpha 1 |
| COL17A1 | BP180, BPAG2 | Collagen, type XVII, alpha 1 |
| COL18A1 | KNO, KNO1, KS | Collagen, type XVIII, alpha 1 |
| COL1A1 | OI4 | Collagen, type I, alpha 1 |
| COL1A2 | OI4 | Collagen, type I, alpha 2 |
| COL4A2 | DKFZp686I14213, FLJ22259 | Collagen, type IV, alpha 2 |
| COL4A3 | | Collagen, type IV, alpha 3 (Goodpasture |
| | | antigen) |
| COL4A4 | CA44 | Collagen, type IV, alpha 4 |
| COL4A5 | ASLN, ATS | Collagen, type IV, alpha 5 |
| COL6A1 | | Collagen, type VI, alpha 1 |
| COX17 | | COX17 cytochrome c oxidase copper |
| | | chaperone |
| CP | | Ceruloplasmin (ferroxidase) |
| CRABP1 | CRABP, CRABP-I, CRABPI, | Cellular retinoic acid binding protein 1 |
| | RBP5 | |
| CRADD | RAIDD | CASP2 and RIPK1 domain containing |
| | | adaptor with death domain |
| CREBBP | CBP, KAT3A, RSTS, RTS | CREB binding protein |
| CRP | PTX1 | C-reactive protein, pentraxin-related |
| CRYAB | CRYA2, HSPB5 | Crystallin, alpha B |
| CSE1L | CAS, CSE1, XPO2 | CSE1 chromosome segregation 1-like |
| | | (yeast) |
| CSF1 | M-CSF, MCSF, MGC31930 | Colony stimulating factor 1 (macrophage) |
| CSF1R | C-FMS, CD115, CSFR, FMS | Colony stimulating factor 1 receptor |
| CSF2 | GM-CSF, GMCSF | Colony stimulating factor 2 (granulocyte- |
| | | macrophage) |
| CSF2RA | CD116, CSF2R | Colony stimulating factor 2 receptor, alpha, |
| | | low-affinity (granulocyte-macrophage) |
| CSF3 | C17orf33, G-CSF, GCSF, | Colony stimulating factor 3 (granulocyte) |
| | MGC45931 | |
| CSN1S1 | CASA, CSN1 | Casein alpha s1 |
| CSNK1E | CKIE, CKIepsilon, HCKIE | Casein kinase 1, epsilon |
| CSNK2A1 | | Casein kinase 2, alpha 1 polypeptide |
| CSNK2A2 | CSNK2A1 | Casein kinase 2, alpha prime polypeptide |
| CSNK2B | | Casein kinase 2, beta polypeptide |
| CST3 | | Cystatin C |
| CST6 | | Cystatin E/M |
| CSTA | STF1, STFA | Cystatin A (stefin A) |
| CSTB | CST6, EPM1, PME, STFB | Cystatin B (stefin B) |
| CTAG1A | ESO1, LAGE2A | Cancer/testis antigen 1A |
| CTAG1B | CT6.1, CTAG, CTAG1, ESO1, | Cancer/testis antigen 1B |
| | LAGE2A, LAGE2B, NY-ESO-1 | |
| CTAG2 | CAMEL, CT6.2a, CT6.2b, | Cancer/testis antigen 2 |
| | ESO2, LAGE-1, LAGE-1a, | |
| | LAGE-1b, LAGE1, | |
| | MGC138724, MGC3803 | |
| CTGF | CCN2, IGFBP8 | Connective tissue growth factor |
| CTNNB1 | armadillo, beta-catenin, CTNNB | Catenin (cadherin-associated protein), beta |
| | | 1, 88 kDa |
| CTNNBL1 | C20orf33, FLJ21108, NAP, | Catenin, beta like 1 |
| | NYD-SP19, P14, P14L | |
| CTSB | | Cathepsin B |
| CTSD | CLN10, CPSD | Cathepsin D |
| CTSH | ACC-4, ACC-5, CPSB | Cathepsin H |
| CTSL | CTSL1, FLJ31037 | Cathepsin L |
| CUL2 | | Cullin 2 |
| CUL5 | VACM-1 | Cullin 5 |
| CXCL1 | FSP, GRO1, GROa, MGSA, | Chemokine (C-X-C motif) ligand 1 |
| | MGSA-a, NAP-3, SCYB1 | (melanoma growth stimulating activity, |
| | | alpha) |
| CXCL10 | C7, crg-2, gIP-10, IFI10, INP10, | Chemokine (C-X-C motif) ligand 10 |
| | IP-10, mob-1, SCYB10 | |
| CXCL13 | ANGIE, ANGIE2, BCA-1, BLC, | Chemokine (C-X-C motif) ligand 13 |
| | BLR1L, SCYB13 | |
| CXCL2 | CINC-2a, GRO2, GROb, | Chemokine (C-X-C motif) ligand 2 |
| | MGSA-b, MIP-2a, SCYB2 | |
| CXCL5 | ENA-78, SCYB5 | Chemokine (C-X-C motif) ligand 5 |
| CXCL8 | 3-10C, AMCF-I, b-ENAP, GCP- | Chemokine (C-X-C motif) ligand 8 |
| | 1, GCP1, IL-8, IL8, K60, LECT, | |
| | LUCT, LYNAP, MDNCF, | |
| | MONAP, NAF, NAP-1, NAP1, | |
| | SCYB8, TSG-1 | |
| CXCL9 | CMK, crg-10, Humig, MIG, | Chemokine (C-X-C motif) ligand 9 |
| | SCYB9 | |
| CXCR1 | CD181, CDw128a, CKR-1, | Chemokine (C-X-C motif) receptor 1 |
| | CMKAR1, IL8RA | |
| CXCR2 | CD182, CMKAR2, IL8RB | Chemokine (C-X-C motif) receptor 2 |
| CXCR4 | CD184, D2S201E, fusin, HM89, | Chemokine (C-X-C motif) receptor 4 |
| | HSY3RR, LESTR, NPY3R, | |
| | NPYR, NPYY3R | |
| CYB5R3 | DIA1 | Cytochrome b5 reductase 3 |
| CYP19A1 | ARO, ARO1, aromatase, CPV1, | Cytochrome P450, family 19, subfamily A, |
| | CYAR, CYP19, P-450AROM | polypeptide 1 |
| CYP1A2 | CP12, P3-450 | Cytochrome P450, family 1, subfamily A, |
| | | polypeptide 2 |
| CYP2C19 | CPCJ, CYP2C, P450IIC19 | Cytochrome P450, family 2, subfamily C, |
| | | polypeptide 19 |
| CYP2E1 | CYP2E | Cytochrome P450, family 2, subfamily E, |
| | | polypeptide 1 |
| CYP3A4 | CYP3A3 | Cytochrome P450, family 3, subfamily A, |
| | | polypeptide 4 |
| CYP3A5 | CP35, P450PCN3, PCN3 | Cytochrome P450, family 3, subfamily A, |
| | | polypeptide 5 |
| DAD1 | OST2 | Defender against cell death 1 |
| DAPK1 | DAPK | Death-associated protein kinase 1 |
| DAXX | DAP6 | Death-domain associated protein |
| DBI | ACBD1, ACBP | Diazepam binding inhibitor (GABA |
| | | receptor modulator, acyl-CoA binding |
| | | protein) |
| DCC | IGDCC1, NTN1R1 | DCC netrin 1 receptor |
| DCDC1 | | Doublecortin domain containing 1 |
| DCN | DSPG2, SLRR1B | Decorin |
| DDB2 | DDBB, FLJ34321, UV-DDB2 | Damage-specific DNA binding protein 2, |
| | | 48 kDa |
| DDIT3 | CHOP, CHOP10, GADD153 | DNA-damage-inducible transcript 3 |
| DEFA1 | DEF1, DEFA2, HNP-1, MRS | Defensin, alpha 1 |
| DEFA1B | | Defensin, alpha 1B |
| DEFA3 | DEF3, HNP-3 | Defensin, alpha 3, neutrophil-specific |
| DEK | D6S231E | DEK proto-oncogene |
| DES | CMD1I, CSM1, CSM2 | Desmin |
| DHFR | | Dihydrofolate reductase |
| DIAPH3 | AN, AUNA1, DRF3, FLJ34705, | Diaphanous-related formin 3 |
| | NSDAN | |
| DLC1 | ARHGAP7, DLC-1, HP, p122- | DLC1 Rho GTPase activating protein |
| | RhoGAP, STARD12 | |
| DNAJC2 | MPHOSPH11, MPP11, ZRF1, | DnaJ (Hsp40) homolog, subfamily C, |
| | ZUO1, zuotin | member 2 |
| DST | BP240, BPA, BPAG1, CATX- | Dystonin |
| | 15, FLJ13425, FLJ21489, | |
| | FLJ30627, FLJ32235, | |
| | KIAA0728, MACF2 | |
| DUSP1 | CL100, HVH1, MKP-1, PTPN10 | Dual specificity phosphatase 1 |
| DUSP14 | MKP-L, MKP6 | Dual specificity phosphatase 14 |
| DUSP4 | HVH2, MKP-2, TYP | Dual specificity phosphatase 4 |
| DVL3 | KIAA0208 | Dishevelled segment polarity protein 3 |
| DYNLL1 | DLC1, DLC8, DNCL1, hdlc1, | Dynein, light chain, LC8-type 1 |
| | LC8, PIN | |
| DYRK2 | | Dual-specificity tyrosine-(Y)- |
| | | phosphorylation regulated kinase 2 |
| E2F1 | RBBP3, RBP3 | E2F transcription factor 1 |
| E2F3 | | E2F transcription factor 3 |
| E2F5 | | E2F transcription factor 5, p130-binding |
| EBAG9 | EB9, RCAS1 | Estrogen receptor binding site associated, |
| | | antigen, 9 |
| EDN1 | ET1 | Endothelin 1 |
| EEF2 | EEF-2, EF2 | Eukaryotic translation elongation factor 2 |
| EFNA1 | ECKLG, EPLG1, LERK1, | Ephrin-A1 |
| | TNFAIP4 | |
| EFNA2 | ELF-1, EPLG6, LERK6 | Ephrin-A2 |
| EFNA5 | AF1, EPLG7, LERK7 | Ephrin-A5 |
| EFNB1 | CFNS, Elk-L, EPLG2, LERK2 | Ephrin-B1 |
| EFNB2 | EPLG5, Htk-L, HTKL, LERK5, | Ephrin-B2 |
| | MGC126226, MGC126227, | |
| | MGC126228 | |
| EFNB3 | EPLG8, LERK-8 | Ephrin-B3 |
| EGF | | Epidermal growth factor |
| EGFR | ERBB, ERBB1 | Epidermal growth factor receptor |
| EGR1 | AT225, G0S30, KROX-24, | Early growth response 1 |
| | NGFI-A, TIS8, ZIF-268, | |
| | ZNF225 | |
| EI24 | EPG4, PIG8, TP53I8 | Etoposide induced 2.4 |
| EIF3H | eIF3-gamma, eIF3-p40, eIF3h, | Eukaryotic translation initiation factor 3, |
| | EIF3S3 | subunit H |
| EIF4E | EIF4E1, EIF4EL1, EIF4F | Eukaryotic translation initiation factor 4E |
| EIF4EBP1 | 4E-BP1, PHAS-I | Eukaryotic translation initiation factor 4E |
| | | binding protein 1 |
| EIF4G1 | EIF4F, EIF4G, p220, PARK18 | Eukaryotic translation initiation factor 4 |
| | | gamma, 1 |
| EIF4H | KIAA0038, WBSCR1, WSCR1 | Eukaryotic translation initiation factor 4H |
| EIF5A | EIF-5A, EIF5A1, MGC104255, | Eukaryotic translation initiation factor 5A |
| | MGC99547 | |
| ELANE | ELA2, HLE, HNE, NE | Elastase, neutrophil expressed |
| ELK3 | ERP, NET, SAP2 | ELK3, ETS-domain protein (SRF |
| | | accessory protein 2) |
| ENC1 | ENC-1, KLHL37, NRPB, | Ectodermal-neural cortex 1 (with BTB |
| | PIG10, TP53I10 | domain) |
| ENG | CD105, END, HHT1, ORW, | Endoglin |
| | ORW1 | |
| ENO1 | ENO1L1, MBP-1, MPB1, PPH | Enolase 1, (alpha) |
| ENO2 | | Enolase 2 (gamma, neuronal) |
| ENPP2 | ATX, PD-IALPHA, PDNP2 | Ectonucleotide |
| | | pyrophosphatase/phosphodiesterase 2 |
| EPAS1 | bHLHe73, HIF2A, HLF, MOP2, | Endothelial PAS domain protein 1 |
| | PASD2 | |
| EPCAM | 17-1A, 323/A3, CD326, CO- | Epithelial cell adhesion molecule |
| | 17A, EGP-2, EGP34, EGP40, | |
| | Ep-CAM, ESA, GA733-2, | |
| | HEA125, KS1/4, KSA, Ly74, | |
| | M4S1, MH99, MIC18, MK-1, | |
| | MOC31, TACST-1, TACSTD1, | |
| | TROP1 | |
| EPHA1 | EPH, EPHT, EPHT1 | EPH receptor A1 |
| EPHA2 | ECK | EPH receptor A2 |
| EPHA3 | ETK, ETK1, HEK, HEK4, | EPH receptor A3 |
| | TYRO4 | |
| EPHA4 | Hek8, TYRO1 | EPH receptor A4 |
| EPHA7 | Hek11 | EPH receptor A7 |
| EPHA8 | EEK, Hek3 | EPH receptor A8 |
| EPHB2 | DRT, EPHT3, ERK, Hek5, | EPH receptor B2 |
| | Tyro5 | |
| EPHB3 | ETK2, Hek2, Tyro6 | EPH receptor B3 |
| EPHB4 | HTK, Tyro11 | EPH receptor B4 |
| EPHX1 | EPHX | Epoxide hydrolase 1, microsomal |
| | | (xenobiotic) |
| EPO | EP | Erythropoietin |
| EPOR | | Erythropoietin receptor |
| ERBB2 | CD340, HER-2, HER2, NEU, | V-erb-b2 avian erythroblastic leukemia |
| | NGL | viral oncogene homolog 2 |
| ERBB3 | HER3, LCCS2 | V-erb-b2 avian erythroblastic leukemia |
| | | viral oncogene homolog 3 |
| ERBB4 | ALS19 | V-erb-b2 avian erythroblastic leukemia |
| | | viral oncogene homolog 4 |
| ERCC1 | RAD10 | Excision repair cross-complementation |
| | | group 1 |
| ERCC2 | EM9, MAG, MGC102762, | Excision repair cross-complementation |
| | MGC126218, MGC126219, | group 2 |
| | TFIIH, XPD | |
| ERCC3 | BTF2, GTF2H, RAD25, TFIIH, | Excision repair cross-complementation |
| | XPB | group 3 |
| ERCC4 | FANCQ, RAD1, XPF | Excision repair cross-complementation |
| | | group 4 |
| ERCC5 | ERCM2, XPGC | Excision repair cross-complementation |
| | | group 5 |
| ERCC6 | ARMD5, CKN2, CSB, RAD26 | Excision repair cross-complementation |
| | | group 6 |
| ESR1 | Era, ESR, NR3A1 | Estrogen receptor 1 |
| ESR2 | Erb, NR3A2 | Estrogen receptor 2 (ER beta) |
| ETHE1 | HSCO, YF13H12 | Ethylmalonic encephalopathy 1 |
| ETV4 | E1A-F, E1AF, PEA3 | Ets variant 4 |
| ETV5 | ERM | Ets variant 5 |
| EXT1 | LGCR, LGS, ttv | Exostosin glycosyltransferase 1 |
| EZH2 | ENX-1, EZH1, KMT6, KMT6A | Enhancer of zeste 2 polycomb repressive |
| | | complex 2 subunit |
| EZR | VIL2 | Ezrin |
| F13A1 | F13A | Coagulation factor XIII, A1 polypeptide |
| F13B | FXIIIB | Coagulation factor XIII, B polypeptide |
| F2 | | Coagulation factor II (thrombin) |
| F3 | CD142 | Coagulation factor III (thromboplastin, |
| | | tissue factor) |
| FABP1 | L-FABP | Fatty acid binding protein 1, liver |
| FABP2 | I-FABP | Fatty acid binding protein 2, intestinal |
| FABP4 | A-FABP, aP2 | Fatty acid binding protein 4, adipocyte |
| FABP5 | E-FABP, KFABP, PA-FABP | Fatty acid binding protein 5 (psoriasis- |
| | | associated) |
| FADD | GIG3, MORT1 | Fas (TNFRSF6)-associated via death |
| | | domain |
| FAF1 | CGI-03, hFAF1, HFAF1s, | Fas (TNFRSF6) associated factor 1 |
| | UBXD12, UBXN3A | |
| FAM129A | C1orf24, GIG39, NIBAN | Family with sequence similarity 129, |
| | | member A |
| FAP | DPPIV | Fibroblast activation protein, alpha |
| FAS | APO-1, APT1, CD95, FAS1, | Fas cell surface death receptor |
| | TNFRSF6 | |
| FASLG | APT1LG1, CD178, FasL, | Fas ligand (TNF superfamily, member 6) |
| | TNFSF6 | |
| FASN | FAS, SDR27X1 | Fatty acid synthase |
| FBXO6 | FBG2, FBS2, FBX6, Fbx6b | F-box protein 6 |
| FCER2 | CD23, CD23A, CLEC4J, FCE2 | Fc fragment of IgE, low affinity II, receptor |
| | | for (CD23) |
| FEN1 | FEN-1, MF1, RAD2 | Flap structure-specific endonuclease 1 |
| FES | FPS | FES proto-oncogene, tyrosine kinase |
| FGA | | Fibrinogen alpha chain |
| FGB | | Fibrinogen beta chain |
| FGF1 | AFGF, ECGF, ECGF-beta, | Fibroblast growth factor 1 (acidic) |
| | ECGFA, ECGFB, FGF-alpha, | |
| | FGFA, GLIO703, HBGF1 | |
| FGF17 | FGF-13 | Fibroblast growth factor 17 |
| FGF18 | FGF-18, ZFGF5 | Fibroblast growth factor 18 |
| FGF19 | | Fibroblast growth factor 19 |
| FGF2 | FGFB | Fibroblast growth factor 2 (basic) |
| FGF23 | | Fibroblast growth factor 23 |
| FGF3 | HBGF-3, INT2 | Fibroblast growth factor 3 |
| FGF4 | HBGF-4, HST, HST-1, HSTF1, | Fibroblast growth factor 4 |
| | K-FGF, KFGF | |
| FGF6 | | Fibroblast growth factor 6 |
| FGF7 | KGF | Fibroblast growth factor 7 |
| FGF8 | AIGF | Fibroblast growth factor 8 (androgen- |
| | | induced) |
| FGF9 | | Fibroblast growth factor 9 |
| FGFR1 | BFGFR, CD331, CEK, FLG, | Fibroblast growth factor receptor 1 |
| | FLT2, H2, H3, H4, H5, KAL2, | |
| | N-SAM | |
| FGFR2 | BEK, CD332, CEK3, CFD1, | Fibroblast growth factor receptor 2 |
| | ECT1, JWS, K-SAM, KGFR, | |
| | TK14, TK25 | |
| FGFR3 | ACH, CD333, CEK2, JTK4 | Fibroblast growth factor receptor 3 |
| FGFR4 | CD334, JTK2 | Fibroblast growth factor receptor 4 |
| FGG | | Fibrinogen gamma chain |
| FHIT | AP3Aase, FRA3B | Fragile histidine triad |
| FIGF | VEGF-D, VEGFD | C-fos induced growth factor (vascular |
| | | endothelial growth factor D) |
| FKBP5 | FKBP51, FKBP54, P54, PPIase, | FK506 binding protein 5 |
| | Ptg-10 | |
| FKBP8 | FKBP38, FKBPr38 | FK506 binding protein 8, 38 kDa |
| FLT1 | FLT, VEGFR1 | Fms-related tyrosine kinase 1 |
| FLT4 | PCL, VEGFR3 | Fms-related tyrosine kinase 4 |
| FMO5 | | Flavin containing monooxygenase 5 |
| FN1 | CIG, FINC, GFND2, LETS, | Fibronectin 1 |
| | MSF | |
| FOLH1 | FOLH, GCP2, GCPII, | Folate hydrolase (prostate-specific |
| | NAALAD1, NAALAdase, PSM, | membrane antigen) 1 |
| | PSMA | |
| FOS | AP-1, c-fos | FBJ murine osteosarcoma viral oncogene |
| | | homolog |
| FOSL1 | fra-1 | FOS-like antigen 1 |
| FOXJ1 | FKHL13, HFH-4, HFH4 | Forkhead box J1 |
| FOXM1 | FKHL16, HFH-11, HNF-3, INS- | Forkhead box M1 |
| | 1, MPHOSPH2, MPP2, TGT3, | |
| | trident | |
| FOXO1 | FKH1, FKHR, FOXO1A | Forkhead box O1 |
| FOXO3 | AF6q21, FKHRL1, FOXO2, | Forkhead box O3 |
| | FOXO3A | |
| FOXQ1 | HFH1 | Forkhead box Q1 |
| FSCN1 | FLJ38511, p55, SNL | Fascin actin-bundling protein 1 |
| FSHB | | Follicle stimulating hormone, beta |
| | | polypeptide |
| FST | FS | Follistatin |
| FTH1 | FHC, FTH, FTHL6, PIG15, | Ferritin, heavy polypeptide 1 |
| | PLIF | |
| FTL | MGC71996, NBIA3 | Ferritin, light polypeptide |
| FZD1 | DKFZp564G072 | Frizzled class receptor 1 |
| FZD2 | | Frizzled class receptor 2 |
| G6PD | G6PD1 | Glucose-6-phosphate dehydrogenase |
| GADD45A | DDIT1, GADD45 | Growth arrest and DNA-damage-inducible, |
| | | alpha |
| GADD45G | CR6, DDIT2, GADD45gamma, | Growth arrest and DNA-damage-inducible, |
| | GRP17 | gamma |
| GAS1 | | Growth arrest-specific 1 |
| GAST | GAS | Gastrin |
| GATA3 | HDR | GATA binding protein 3 |
| GCLM | GLCLR | Glutamate-cysteine ligase, modifier subunit |
| GDF15 | MIC-1, MIC1, NAG-1, PDF, | Growth differentiation factor 15 |
| | PLAB, PTGFB | |
| GDNF | ATF1, ATF2, HFB1-GDNF | Glial cell derived neurotrophic factor |
| GH1 | GH, GH-N, GHN, hGH-N | Growth hormone 1 |
| GH2 | GH-V, GH2, GHL, GHV, hGH- | Growth hormone 2 |
| | V | |
| GJA1 | CX43, GJAL, ODD, ODDD, | Gap junction protein, alpha 1, 43 kDa |
| | ODOD, SDTY3 | |
| GJB5 | CX31.1 | Gap junction protein, beta 5, 31.1 kDa |
| GLO1 | GLOD1 | Glyoxalase I |
| GMNN | Gem | Geminin, DNA replication inhibitor |
| GNAS | GNAS1, GNASXL, GPSA, | GNAS complex locus |
| | NESP, NESP55, SCG6 | |
| GPA33 | A33 | Glycoprotein A33 (transmembrane) |
| GPC3 | DGSX, OCI-5, SDYS, SGB, | Glypican 3 |
| | SGBS, SGBS1 | |
| GPI | AMF, NLK | Glucose-6-phosphate isomerase |
| GPX1 | | Glutathione peroxidase 1 |
| GPX2 | GSHPX-GI | Glutathione peroxidase 2 (gastrointestinal) |
| GRB10 | | Growth factor receptor-bound protein 10 |
| GRB2 | NCKAP2 | Growth factor receptor-bound protein 2 |
| GRB7 | | Growth factor receptor-bound protein 7 |
| GSK3A | | Glycogen synthase kinase 3 alpha |
| GSN | DKFZp313L0718 | Gelsolin |
| GSR | | Glutathione reductase |
| GSTM1 | GST1, H-B, MU | Glutathione S-transferase mu 1 |
| GSTM3 | GST5 | Glutathione S-transferase mu 3 (brain) |
| GSTP1 | FAEES3, GST3, GSTP | Glutathione S-transferase pi 1 |
| HDAC10 | DKFZP761B039 | Histone deacetylase 10 |
| HDAC2 | RPD3, YAF1 | Histone deacetylase 2 |
| HDAC5 | FLJ90614, KIAA0600, NY-CO-9 | Histone deacetylase 5 |
| HGF | DFNB39, F-TCF, HGFB, HPTA, | Hepatocyte growth factor (hepapoietin A; |
| | SF | scatter factor) |
| HGFAC | HGFA, HGFAP | HGF activator |
| HIF1A | bHLHe78, HIF-1alpha, HIF1, | Hypoxia inducible factor 1, alpha subunit |
| | MOP1, PASD8 | (basic helix-loop-helix transcription factor) |
| HIP1R | FLJ14000, HIP12, HIP3, | Huntingtin interacting protein 1 related |
| | ILWEQ, KIAA0655 | |
| HIST1H2AC | H2AFL | Histone cluster 1, H2ac |
| HK1 | | Hexokinase 1 |
| HK2 | | Hexokinase 2 |
| HLA-G | | Major histocompatibility complex, class I, |
| | | G |
| HMGA1 | HMGIY | High mobility group AT-hook 1 |
| HMGA2 | BABL, HMGIC, LIPO | High mobility group AT-hook 2 |
| HMOX1 | bK286B10, HO-1 | Heme oxygenase (decycling) 1 |
| HOXA5 | HOX1, HOX1C | Homeobox A5 |
| HOXA9 | HOX1, HOX1G | Homeobox A9 |
| HP | | Haptoglobin |
| HPGD | SDR36C1 | Hydroxyprostaglandin dehydrogenase 15- |
| | | (NAD) |
| HPN | TMPRSS1 | Hepsin |
| HRAS | HRAS1 | Harvey rat sarcoma viral oncogene |
| | | homolog |
| HSF1 | HSTF1 | Heat shock transcription factor 1 |
| HSP90AA1 | FLJ31884, Hsp89, Hsp90, | Heat shock protein 90 kDa alpha |
| | HSP90N, HSPC1, HSPCA | (cytosolic), class A member 1 |
| HSP90AB1 | HSPC2, HSPCB | Heat shock protein 90 kDa alpha |
| | | (cytosolic), class B member 1 |
| HSP90B1 | GP96, GRP94, TRA1 | Heat shock protein 90 kDa beta |
| | | (Grp94), member 1 |
| HSPA1A | HSP70-1, HSPA1 | Heat shock 70 kDa protein 1A |
| HSPA1B | HSP70-2 | Heat shock 70 kDa protein 1B |
| HSPA1L | HSP70-HOM, hum70t | Heat shock 70 kDa protein 1-like |
| HSPA2 | | Heat shock 70 kDa protein 2 |
| HSPA4 | HS24/P52, HSPH2 | Heat shock 70 kDa protein 4 |
| HSPA8 | HSC70, HSC71, HSP73, | Heat shock 70 kDa protein 8 |
| | HSPA10 | |
| HSPB1 | Hs.76067, Hsp25, HSP27, | Heat shock 27 kDa protein 1 |
| | HSP28 | |
| HSPD1 | GROEL, HSP60, SPG13 | Heat shock 60 kDa protein 1 (chaperonin) |
| HSPE1 | CPN10, GROES | Heat shock 10 kDa protein 1 |
| HSPH1 | HSP105A, HSP105B, | Heat shock 105 kDa/110 kDa protein 1 |
| | KIAA0201, NY-CO-25 | |
| IBSP | BSP, BSP-II, SP-II | Integrin-binding sialoprotein |
| ICAM1 | BB2, CD54 | Intercellular adhesion molecule 1 |
| ID1 | bHLHb24, dJ857M17.1.2 | Inhibitor of DNA binding 1, dominant |
| | | negative helix-loop-helix protein |
| ID2 | bHLHb26, GIG8 | Inhibitor of DNA binding 2, dominant |
| | | negative helix-loop-helix protein |
| ID3 | bHLHb25, HEIR-1 | Inhibitor of DNA binding 3, dominant |
| | | negative helix-loop-helix protein |
| IDO1 | IDO, INDO | Indoleamine 2,3-dioxygenase 1 |
| IFNA1 | IFL, IFN, IFN-ALPHA, IFN- | Interferon, alpha 1 |
| | alphaD, IFNA13, IFNA@ | |
| IFNA13 | | Interferon, alpha 13 |
| IFNAR1 | IFNAR, IFRC | Interferon (alpha, beta and omega) receptor 1 |
| IFNAR2 | IFNABR | Interferon (alpha, beta and omega) receptor 2 |
| IFNB1 | IFB, IFF, IFNB | Interferon, beta 1, fibroblast |
| IFNG | | Interferon, gamma |
| IGF1 | IGF-I, IGF1A, IGFI | Insulin-like growth factor 1 (somatomedin C) |
| IGF1R | CD221, IGFIR, IGFR, JTK13, | Insulin-like growth factor 1 receptor |
| | MGC18216 | |
| IGF2 | C11orf43, FLJ44734, IGF-II | Insulin-like growth factor 2 |
| IGF2R | CD222, CIMPR, M6P-R, MPR1, | Insulin-like growth factor 2 receptor |
| | MPRI | |
| IGFBP2 | IBP2 | Insulin-like growth factor binding protein |
| | | 2, 36 kDa |
| IGFBP3 | BP-53, IBP3 | Insulin-like growth factor binding protein 3 |
| IL10 | CSIF, IL-10, IL10A, TGIF | Interleukin 10 |
| IL11 | AGIF, IL-11 | Interleukin 11 |
| IL12A | CLMF, IL-12A, NFSK, NKSF1, | Interleukin 12A |
| | p35 | |
| IL13 | ALRH, BHR1, IL-13, | Interleukin 13 |
| | MGC116786, MGC116788, | |
| | MGC116789, P600 | |
| IL13RA2 | CD213a2, CT19, IL-13R, | Interleukin 13 receptor, alpha 2 |
| | IL13BP | |
| IL15 | IL-15, MGC9721 | Interleukin 15 |
| IL16 | FLJ16806, FLJ42735, | Interleukin 16 |
| | HsT19289, IL-16, LCF, prIL-16 | |
| IL17A | CTLA8, IL-17, IL-17A, IL17 | Interleukin 17A |
| IL17B | IL-17B, IL-20, MGC138900, | Interleukin 17B |
| | MGC138901, NIRF, ZCYTO7 | |
| IL18 | IGIF, IL-18, IL-1g, IL1F4 | Interleukin 18 |
| IL1A | IL-1A, IL1, IL1-ALPHA, IL1F1 | Interleukin 1, alpha |
| IL1B | IL-1B, IL1-BETA, IL1F2 | Interleukin 1, beta |
| IL1R1 | CD121A, D2S1473, IL1R, | Interleukin 1 receptor, type I |
| | IL1RA | |
| IL1R2 | CD121b, IL1RB | Interleukin 1 receptor, type II |
| IL1RN | ICIL-1RA, IL-1RN, IL1F3, | Interleukin 1 receptor antagonist |
| | IL1RA, IRAP, MGC10430 | |
| IL2 | IL-2, TCGF | Interleukin 2 |
| IL24 | C49A, FISP, IL-24, IL10B, mda- | Interleukin 24 |
| | 7, Mob-5, ST16 | |
| IL2RA | CD25, IDDM10, IL2R | Interleukin 2 receptor, alpha |
| IL2RB | CD122, IL15RB | Interleukin 2 receptor, beta |
| IL2RG | CD132, CIDX, IMD4, SCIDX1 | Interleukin 2 receptor, gamma |
| IL4 | BCGF-1, BCGF1, BSF1, IL-4, | Interleukin 4 |
| | MGC79402 | |
| IL4R | CD124 | Interleukin 4 receptor |
| IL5 | EDF, IL-5, TRF | Interleukin 5 |
| IL6 | BSF2, HGF, HSF, IFNB2, IL-6 | Interleukin 6 |
| IL6R | CD126 | Interleukin 6 receptor |
| IL6ST | CD130, GP130 | Interleukin 6 signal transducer |
| IL7 | IL-7 | Interleukin 7 |
| IL9 | HP40, IL-9, P40 | Interleukin 9 |
| ILF3 | DRBP76, MPHOSPH4, MPP4, | Interleukin enhancer binding factor 3, |
| | NF90, NFAR-1 | 90 kDa |
| ILK | | Integrin-linked kinase |
| INHBA | | Inhibin, beta A |
| INHBB | | Inhibin, beta B |
| INS | IDDM1, IDDM2 | Insulin |
| IRF1 | MAR | Interferon regulatory factor 1 |
| IRF4 | LSIRF, MUM1 | Interferon regulatory factor 4 |
| ITGA1 | CD49a, VLA1 | Integrin, alpha 1 |
| ITGA2 | CD49B | Integrin, alpha 2 (CD49B, alpha 2 subunit |
| | | of VLA-2 receptor) |
| ITGA2B | CD41, CD41B, GP2B, PPP1R93 | Integrin, alpha 2b (platelet glycoprotein IIb |
| | | of IIb/IIIa complex, antigen CD41) |
| ITGA3 | CD49c, GAP-B3, MSK18, VCA- | Integrin, alpha 3 (antigen CD49C, alpha 3 |
| | 2, VLA3a | subunit of VLA-3 receptor) |
| ITGA4 | CD49D | Integrin, alpha 4 (antigen CD49D, alpha 4 |
| | | subunit of VLA-4 receptor) |
| ITGA5 | CD49e, FNRA | Integrin, alpha 5 (fibronectin receptor, |
| | | alpha polypeptide) |
| ITGA6 | CD49f | Integrin, alpha 6 |
| ITGAM | CD11B, CR3A, MAC-1 | Integrin, alpha M (complement component |
| | | 3 receptor 3 subunit) |
| ITGAV | CD51, MSK8, VNRA, VTNR | Integrin, alpha V |
| ITGB1 | CD29, FNRB, GPIIA, MDF2, | Integrin, beta 1 (fibronectin receptor, beta |
| | MSK12 | polypeptide, antigen CD29 includes MDF2, |
| | | MSK12) |
| ITGB3 | CD61, GP3A, GPIIIa | Integrin, beta 3 (platelet glycoprotein IIIa, |
| | | antigen CD61) |
| ITGB4 | CD104 | Integrin, beta 4 |
| ITGB5 | | Integrin, beta 5 |
| ITGB6 | | Integrin, beta 6 |
| ITGB8 | | Integrin, beta 8 |
| ITIH4 | H4P, IHRP, ITIHL1 | Inter-alpha-trypsin inhibitor heavy chain |
| | | family, member 4 |
| JKAMP | C14orf100, CDA06, HSPC213, | JNK1/MAPK8-associated membrane |
| | HSPC327, JAMP | protein |
| JTB | hJT | Jumping translocation breakpoint |
| JUN | AP-1, c-Jun | Jun proto-oncogene |
| JUND | AP-1 | Jun D proto-oncogene |
| JUP | CTNNG, DP3, DPIII, PDGB, | Junction plakoglobin |
| | PKGB | |
| KAT2B | GCN5, GCN5L, P/CAF, PCAF | K(lysine) acetyltransferase 2B |
| KDR | CD309, FLK1, VEGFR, | Kinase insert domain receptor (a type III |
| | VEGFR2 | receptor tyrosine kinase) |
| KIF2A | HK2, KIF2 | Kinesin heavy chain member 2A |
| KIF2C | CT139, KNSL6, MCAK | Kinesin family member 2C |
| KISS1 | | KiSS-1 metastasis-suppressor |
| KIT | C-Kit, CD117, PBT, SCFR | V-kit Hardy-Zuckerman 4 feline sarcoma |
| | | viral oncogene homolog |
| KITLG | FPH2, Kitl, KL-1, MGF, SCF, | KIT ligand |
| | SF | |
| KLF4 | EZF, GKLF | Kruppel-like factor 4 (gut) |
| KLF5 | BTEB2, CKLF, IKLF | Kruppel-like factor 5 (intestinal) |
| KLK10 | NES1, PRSSL1 | Kallikrein-related peptidase 10 |
| KLK11 | PRSS20, TLSP | Kallikrein-related peptidase 11 |
| KLK13 | KLK-L4 | Kallikrein-related peptidase 13 |
| KLK14 | KLK-L6 | Kallikrein-related peptidase 14 |
| KLK15 | ACO, HSRNASPH, prostinogen | Kallikrein-related peptidase 15 |
| KLK2 | | Kallikrein-related peptidase 2 |
| KLK3 | APS, PSA | Kallikrein-related peptidase 3 |
| KLK4 | EMSP, EMSP1, KLK-L1, | Kallikrein-related peptidase 4 |
| | PRSS17, PSTS | |
| KLK5 | KLK-L2, SCTE | Kallikrein-related peptidase 5 |
| KLK6 | Bssp, Klk7, neurosin, PRSS18, | Kallikrein-related peptidase 6 |
| | PRSS9 | |
| KLK7 | PRSS6, SCCE | Kallikrein-related peptidase 7 |
| KLK8 | HNP, neuropsin, ovasin, | Kallikrein-related peptidase 8 |
| | PRSS19, TADG14 | |
| KLRK1 | CD314, D12S2489E, KLR, | Killer cell lectin-like receptor subfamily K, |
| | NKG2-D, NKG2D | member 1 |
| KRAS | KRAS1, KRAS2 | Kirsten rat sarcoma viral oncogene |
| | | homolog |
| KRT13 | CK13, K13, MGC161462, | Keratin 13 |
| | MGC3781 | |
| KRT14 | EBS3, EBS4 | Keratin 14 |
| KRT15 | CK15, K15, K1CO | Keratin 15 |
| KRT17 | PCHC1 | Keratin 17 |
| KRT18 | | Keratin 18 |
| KRT19 | CK19, K19, K1CS, MGC15366 | Keratin 19 |
| KRT4 | CK4, CYK4, K4 | Keratin 4 |
| KRT8 | CARD2, CK8, CYK8, K2C8, | Keratin 8 |
| | K8, KO | |
| LALBA | LYZL7 | Lactalbumin, alpha- |
| LAMB1 | CLM | Laminin, beta 1 |
| LAMC1 | LAMB2 | Laminin, gamma 1 (formerly LAMB2) |
| LCN1 | MGC71975, PMFA, TLC, TP, | Lipocalin 1 |
| | VEGP | |
| LDHA | | Lactate dehydrogenase A |
| LEP | OB, OBS | Leptin |
| LGALS3 | GALIG, LGALS2, MAC-2 | Lectin, galactoside-binding, soluble, 3 |
| LGALS3BP | 90K, BTBD17B, CyCAP, gp90, | Lectin, galactoside-binding, soluble, 3 |
| | M2BP, MAC-2-BP, | binding protein |
| | TANGO10B | |
| LGALS4 | GAL4 | Lectin, galactoside-binding, soluble, 4 |
| LGI1 | EPITEMPIN, EPT, ETL1, | Leucine-rich, glioma inactivated 1 |
| | IB1099 | |
| LGMN | LGMN1, PRSC1 | Legumain |
| LHB | CGB4, hLHB, LSH-B | Luteinizing hormone beta polypeptide |
| LHX1 | LIM-1, LIM1 | LIM homeobox 1 |
| LIF | CDF, DIA, HILDA | Leukemia inhibitory factor |
| LIG4 | | Ligase IV, DNA, ATP-dependent |
| LIMK1 | LIMK | LIM domain kinase 1 |
| LMNA | CMD1A, HGPS, LGMD1B, | Lamin A/C |
| | LMN1, LMNL1, PRO1 | |
| LRP1B | LRP-DIT, LRPDIT | Low density lipoprotein receptor-related |
| | | protein 1B |
| LRP6 | ADCAD2 | Low density lipoprotein receptor-related |
| | | protein 6 |
| LTA | LT, TNFB, TNFSF1 | Lymphotoxin alpha |
| LTA4H | | Leukotriene A4 hydrolase |
| LTB | p33, TNFC, TNFSF3 | Lymphotoxin beta (TNF superfamily, |
| | | member 3) |
| LTBR | D12S370, TNF-R-III, TNFCR, | Lymphotoxin beta receptor (TNFR |
| | TNFR-RP, TNFR2-RP, | superfamily, member 3) |
| | TNFRSF3 | |
| LTF | HLF2 | Lactotransferrin |
| MAD2L1 | HSMAD2, MAD2 | MAD2 mitotic arrest deficient-like 1 |
| | | (yeast) |
| MAD2L2 | MAD2B, POLZ2, REV7 | MAD2 mitotic arrest deficient-like 2 |
| | | (yeast) |
| MAGEA3 | CT1.3, HIP8, HYPD, MAGE3, | Melanoma antigen family A, 3 |
| | MGC14613 | |
| MAGEA4 | CT1.4, MAGE-41, MAGE-X2, | Melanoma antigen family A, 4 |
| | MAGE4, MAGE4A, MAGE4B, | |
| | MGC21336 | |
| MAGEA6 | CT1.6, MAGE6 | Melanoma antigen family A, 6 |
| MAGEB5 | CT3.3, MAGE-B5 | Melanoma antigen family B, 5 |
| MAGEB6 | CT3.4, FLJ40242, MAGE-B6, | Melanoma antigen family B, 6 |
| | MAGEB6A | |
| MAGEC1 | CT7, CT7.1, MAGE-C1, | Melanoma antigen family C, 1 |
| | MGC39366 | |
| MAGEC2 | CT10, MAGE-C2, MAGEE1 | Melanoma antigen family C, 2 |
| MAGEC3 | CT7.2, HCA2, MAGE-C3 | Melanoma antigen family C, 3 |
| MAGED1 | DLXIN-1, NRAGE | Melanoma antigen family D, 1 |
| MAGED2 | 11B6, BCG1, HCA10, JCL-1, | Melanoma antigen family D, 2 |
| | MAGE-D2, MAGED, MGC8386 | |
| MAGI1 | AIP3, BAIAP1, BAP1, MAGI-1, | Membrane associated guanylate kinase, |
| | TNRC19, WWP3 | WW and PDZ domain containing 1 |
| MAP2K1 | MAPKK1, MEK1, PRKMK1 | Mitogen-activated protein kinase kinase 1 |
| MAP2K2 | MEK2, PRKMK2 | Mitogen-activated protein kinase kinase 2 |
| MAP2K4 | JNKK1, MEK4, MKK4, | Mitogen-activated protein kinase kinase 4 |
| | PRKMK4, SERK1 | |
| MAPK1 | ERK, ERK2, MAPK2, p41mapk, | Mitogen-activated protein kinase 1 |
| | PRKM1, PRKM2 | |
| MAPK14 | CSBP1, CSBP2, CSPB1, Mxi2, | Mitogen-activated protein kinase 14 |
| | p38, PRKM14, PRKM15 | |
| MAPK3 | ERK1, p44erk1, p44mapk, | Mitogen-activated protein kinase 3 |
| | PRKM3 | |
| MAPK7 | BMK1, ERK5, PRKM7 | Mitogen-activated protein kinase 7 |
| MAPK8 | JNK, JNK1, PRKM8, SAPK1 | Mitogen-activated protein kinase 8 |
| MAPKAPK2 | | Mitogen-activated protein kinase-activated |
| | | protein kinase 2 |
| MBD1 | CXXC3, PCM1 | Methyl-CpG binding domain protein 1 |
| MBD2 | | Methyl-CpG binding domain protein 2 |
| MBD4 | MED1 | Methyl-CpG binding domain protein 4 |
| MCL1 | BCL2L3, Mcl-1 | Myeloid cell leukemia 1 |
| MCM2 | BM28, CCNL1, cdc19, CDCL1, | Minichromosome maintenance complex |
| | D3S3194, KIAA0030 | component 2 |
| MCM3 | | Minichromosome maintenance complex |
| | | component 3 |
| MCM5 | CDC46 | Minichromosome maintenance complex |
| | | component 5 |
| MCM7 | CDC47, MCM2, PPP1R104 | Minichromosome maintenance complex |
| | | component 7 |
| MDH1 | | Malate dehydrogenase 1, NAD (soluble) |
| MDK | FLJ27379, MK, NEGF2 | Midkine (neurite growth-promoting factor 2) |
| MDM2 | HDM2, MGC5370 | MDM2 proto-oncogene, E3 ubiquitin |
| | | protein ligase |
| MECP2 | MRX16, MRX79, RTT | Methyl CpG binding protein 2 |
| MED1 | CRSP1, CRSP200, DRIP230, | Mediator complex subunit 1 |
| | PBP, PPARBP, PPARGBP, | |
| | RB18A, TRAP220, TRIP2 | |
| MET | HGFR, RCCP2 | MET proto-oncogene, receptor tyrosine |
| | | kinase |
| MFGE8 | BA46, EDIL1, hP47, HsT19888, | Milk fat globule-EGF factor 8 protein |
| | MFG-E8, OAcGD3S, SED1, | |
| | SPAG10 | |
| MGMT | | O-6-methylguanine-DNA |
| | | methyltransferase |
| MIA | CD-RAP | Melanoma inhibitory activity |
| MIF | GIF, GLIF | Macrophage migration inhibitory factor |
| | | (glycosylation-inhibiting factor) |
| MKI67 | MIB-, PPP1R105 | Marker of proliferation Ki-67 |
| MLH1 | COCA2, FCC2, HNPCC, | MutL homolog 1 |
| | HNPCC2 | |
| MLLT11 | AF1Q | Myeloid/lymphoid or mixed-lineage |
| | | leukemia (trithorax homolog, Drosophila); |
| | | translocated to, 11 |
| MME | CALLA, CD10, NEP | Membrane metallo-endopeptidase |
| MMP1 | CLG | Matrix metallopeptidase 1 (interstitial |
| | | collagenase) |
| MMP10 | STMY2 | Matrix metallopeptidase 10 (stromelysin 2) |
| MMP11 | STMY3 | Matrix metallopeptidase 11 (stromelysin 3) |
| MMP12 | HME | Matrix metallopeptidase 12 (macrophage |
| | | elastase) |
| MMP13 | CLG3 | Matrix metallopeptidase 13 (collagenase 3) |
| MMP14 | MT1-MMP | Matrix metallopeptidase 14 (membrane- |
| | | inserted) |
| MMP15 | MT2-MMP, MTMMP2, SMCP-2 | Matrix metallopeptidase 15 (membrane- |
| | | inserted) |
| MMP16 | C8orf57, DKFZp761D112, | Matrix metallopeptidase 16 (membrane- |
| | MT3-MMP | inserted) |
| MMP2 | CLG4, CLG4A, TBE-1 | Matrix metallopeptidase 2 (gelatinase A, |
| | | 72 kDa gelatinase, 72 kDa type IV |
| | | collagenase) |
| MMP3 | STMY, STMY1 | Matrix metallopeptidase 3 (stromelysin 1, |
| | | progelatinase) |
| MMP7 | MPSL1, PUMP-1 | Matrix metallopeptidase 7 (matrilysin, |
| | | uterine) |
| MMP8 | CLG1 | Matrix metallopeptidase 8 (neutrophil |
| | | collagenase) |
| MMP9 | CLG4B | Matrix metallopeptidase 9 (gelatinase B, |
| | | 92 kDa gelatinase, 92 kDa type IV |
| | | collagenase) |
| MPO | | Myeloperoxidase |
| MRE11A | ATLD, MRE11 | MRE11 meiotic recombination 11 homolog |
| | | A (S. cerevisiae) |
| MSH6 | GTBP | MutS homolog 6 |
| MSLN | CAK1, MPF | Mesothelin |
| MSMB | IGBF, MSP, MSPB, PN44, | Microseminoprotein, beta- |
| | PRPS, PSP, PSP-94, PSP57, | |
| | PSP94 | |
| MSR1 | CD204, SCARA1 | Macrophage scavenger receptor 1 |
| MT1A | MT1, MT1S | Metallothionein 1A |
| MT1G | MT1, MT1K | Metallothionein 1G |
| MTA1 | | Metastasis associated 1 |
| MUC1 | ADMCKD, ADMCKD1, | Mucin 1, cell surface associated |
| | CD227, MCD, MCKD, MCKD1, | |
| | PEM, PUM | |
| MUTYH | MYH | MutY homolog |
| MVP | LRP, VAULT1 | Major vault protein |
| MXI1 | bHLHc11, MAD2, MXD2, MXI | MAX interactor 1, dimerization protein |
| MYBL2 | B-MYB, BMYB | V-myb avian myeloblastosis viral oncogene |
| | | homolog-like 2 |
| MYC | bHLHe39, c-Myc, MYCC | V-myc avian myelocytomatosis viral |
| | | oncogene homolog |
| MYOCD | MYCD | Myocardin |
| MYOD1 | bHLHc1, MYF3, MYOD, PUM | Myogenic differentiation 1 |
| MYOG | bHLHc3, MYF4 | Myogenin (myogenic factor 4) |
| NAGA | D22S674 | N-acetylgalactosaminidase, alpha- |
| NAIP | BIRC1, NLRB1 | NLR family, apoptosis inhibitory protein |
| NAMPT | PBEF, PBEF1 | Nicotinamide phosphoribosyltransferase |
| NAT2 | AAC2 | N-acetyltransferase 2 (arylamine N- |
| | | acetyltransferase) |
| NCAM1 | CD56, NCAM | Neural cell adhesion molecule 1 |
| NCOA3 | ACTR, AIB1, bHLHe42, | Nuclear receptor coactivator 3 |
| | CAGH16, KAT13B, p/CIP, | |
| | RAC3, SRC-3, SRC3, TNRC16, | |
| | TRAM-1 | |
| NDRG1 | CAP43, DRG1, NDR1, RTP, | N-myc downstream regulated 1 |
| | TDD5 | |
| NEDD8 | Nedd-8 | Neural precursor cell expressed, |
| | | developmentally down-regulated 8 |
| NEO1 | HsT17534, IGDCC2, NGN, | Neogenin 1 |
| | NTN1R2 | |
| NFKB1 | KBF1, NF-kappaB, NF-kB1, | Nuclear factor of kappa light polypeptide |
| | NFkappaB, NFKB-p50, p105, | gene enhancer in B-cells 1 |
| | p50 | |
| NFKB2 | LYT-10, NF-kB2, p105, p52 | Nuclear factor of kappa light polypeptide |
| | | gene enhancer in B-cells 2 (p49/p100) |
| NFKBIA | IkappaBalpha, IKBA, MAD-3, | Nuclear factor of kappa light polypeptide |
| | NFKBI | gene enhancer in B-cells inhibitor, alpha |
| NFKBIE | IKBE | Nuclear factor of kappa light polypeptide |
| | | gene enhancer in B-cells inhibitor, epsilon |
| NGF | NGFB | Nerve growth factor (beta polypeptide) |
| NGFR | CD271, p75NTR, TNFRSF16 | Nerve growth factor receptor |
| NKX3-1 | BAPX2, NKX3.1, NKX3A | NK3 homeobox 1 |
| NME1 | NDPKA, NM23, NM23-H1 | NME/NM23 nucleoside diphosphate kinase 1 |
| NME2 | NDPKB, NM23-H2 | NME/NM23 nucleoside diphosphate kinase 2 |
| NOS1 | nNOS, NOS | Nitric oxide synthase 1 (neuronal) |
| NOS2 | HEP-NOS, iNOS, NOS, NOS2A | Nitric oxide synthase 2, inducible |
| NOS3 | ECNOS, eNOS | Nitric oxide synthase 3 (endothelial cell) |
| NOTCH1 | TAN1 | Notch 1 |
| NOTCH2 | | Notch 2 |
| NOTCH3 | CADASIL, CASIL | Notch 3 |
| NQO1 | DHQU, DIA4, DTD, NMOR1, | NAD(P)H dehydrogenase, quinone 1 |
| | QR1 | |
| NR0B1 | AHC, AHCH, DAX1, DSS, | Nuclear receptor subfamily 0, group B, |
| | NR0B1 | member 1 |
| NRG1 | GGF, HGL, HRG, NDF, NRG1- | Neuregulin 1 |
| | IT2 | |
| NRG2 | Don-1, HRG2, NTAK | Neuregulin 2 |
| NRG3 | | Neuregulin 3 |
| NRP1 | CD304, NRP, VEGF165R | Neuropilin 1 |
| NRP2 | VEGF165R2 | Neuropilin 2 |
| NTF3 | NGF2 | Neurotrophin 3 |
| NTF4 | GLC1O, NT-4/5, NTF5 | Neurotrophin 4 |
| NTHL1 | NTH1, OCTS3 | Nth endonuclease III-like 1 (E. coli) |
| NTN1 | NTN1L | Netrin 1 |
| NTRK1 | MTC, TRK, TRKA | Neurotrophic tyrosine kinase, receptor, type 1 |
| NTRK2 | TRKB | Neurotrophic tyrosine kinase, receptor, type 2 |
| NTRK3 | TRKC | Neurotrophic tyrosine kinase, receptor, type 3 |
| NUDT1 | MTH1 | Nudix (nucleoside diphosphate linked |
| | | moiety X)-type motif 1 |
| NUMB | C14orf41 | Numb homolog (Drosophila) |
| OGG1 | HMMH, HOGG1, MUTM, | 8-oxoguanine DNA glycosylase |
| | OGH1 | |
| OR51E2 | PSGR | Olfactory receptor, family 51, subfamily E, |
| | | member 2 |
| ORM1 | | Orosomucoid 1 |
| OSM | MGC20461 | Oncostatin M |
| PAGE4 | CT16.7, GAGEC1, PAGE-4 | P antigen family, member 4 (prostate |
| | | associated) |
| PAPPA | ASBABP2, DIPLA1, IGFBP- | Pregnancy-associated plasma protein A, |
| | 4ase, PAPA, PAPP-A, PAPPA1 | pappalysin 1 |
| PARP1 | ADPRT, PARP, PPOL | Poly (ADP-ribose) polymerase 1 |
| PARVB | CGI-56 | Parvin, beta |
| PAX5 | BSAP | Paired box 5 |
| PAX8 | | Paired box 8 |
| PCNA | | Proliferating cell nuclear antigen |
| PDGFA | PDGF-A, PDGF1 | Platelet-derived growth factor alpha |
| | | polypeptide |
| PDGFB | SIS, SSV | Platelet-derived growth factor beta |
| | | polypeptide |
| PDGFRA | CD140a, PDGFR2 | Platelet-derived growth factor receptor, |
| | | alpha polypeptide |
| PDGFRB | CD140b, JTK12, PDGFR, | Platelet-derived growth factor receptor, |
| | PDGFR1 | beta polypeptide |
| PDZD4 | FLJ34125, KIAA1444, LU1, | PDZ domain containing 4 |
| | PDZK4, PDZRN4L | |
| PF4 | CXCL4, SCYB4 | Platelet factor 4 |
| PGC | | Progastricsin (pepsinogen C) |
| PGF | D12S1900, PGFL, PLGF, PlGF- | Placental growth factor |
| | 2, SHGC-10760 | |
| PGR | NR3C3, PR | Progesterone receptor |
| PHF20 | C20orf104, dJ1121G12.1, | PHD finger protein 20 |
| | TDRD20A | |
| PIGR | | Polymeric immunoglobulin receptor |
| PIK3CA | PI3K | Phosphatidylinositol-4,5-bisphosphate 3- |
| | | kinase, catalytic subunit alpha |
| PIK3R1 | GRB1, p85, p85-ALPHA | Phosphoinositide-3-kinase, regulatory |
| | | subunit 1 (alpha) |
| PIK3R2 | p85, P85B | Phosphoinositide-3-kinase, regulatory |
| | | subunit 2 (beta) |
| PIK3R3 | p55 | Phosphoinositide-3-kinase, regulatory |
| | | subunit 3 (gamma) |
| PIM1 | PIM | Pim-1 proto-oncogene, serine/threonine |
| | | kinase |
| PIM2 | | Pim-2 proto-oncogene, serine/threonine |
| | | kinase |
| PIM3 | | Pim-3 proto-oncogene, serine/threonine |
| | | kinase |
| PIN1 | dod | Peptidylprolyl cis/trans isomerase, NIMA- |
| | | interacting 1 |
| PIP4K2B | PIP5K2B, PIP5KIIB, | Phosphatidylinositol-5-phosphate 4-kinase, |
| | PIP5KIIbeta | type II, beta |
| PKM | OIP3, PK3, PKM2, THBP1 | Pyruvate kinase, muscle |
| PLAT | | Plasminogen activator, tissue |
| PLAU | UPA, URK | Plasminogen activator, urokinase |
| PLAUR | CD87, UPAR, URKR | Plasminogen activator, urokinase receptor |
| PLG | | Plasminogen |
| PLK1 | PLK | Polo-like kinase 1 |
| PLP1 | GPM6C, PLP, SPG2 | Proteolipid protein 1 |
| PMEPA1 | STAG1, TMEPAI | Prostate transmembrane protein, androgen |
| | | induced 1 |
| PML | MYL, RNF71, TRIM19 | Promyelocytic leukemia |
| PMP22 | GAS-3, HNPP, Sp110 | Peripheral myelin protein 22 |
| PNMT | PENT | Phenylethanolamine N-methyltransferase |
| POMC | ACTH, CLIP, LPH, MSH, NPP, | Proopiomelanocortin |
| | POC | |
| PON1 | ESA, PON | Paraoxonase 1 |
| POSTN | OSF-2, periostin, PN | Periostin, osteoblast specific factor |
| POU2F2 | OCT2, OTF2 | POU class 2 homeobox 2 |
| PPA2 | FLJ20459 | Pyrophosphatase (inorganic) 2 |
| PPARG | NR1C3, PPARG1, PPARG2, | Peroxisome proliferator-activated receptor |
| | PPARgamma | gamma |
| PPARGC1A | PGC1, PGC1A, PPARGC1 | Peroxisome proliferator-activated receptor |
| | | gamma, coactivator 1 alpha |
| PPM1D | PP2C-DELTA, Wip1 | Protein phosphatase, Mg2+/Mn2+ |
| | | dependent, 1D |
| PPP1R15A | GADD34 | Protein phosphatase 1, regulatory subunit |
| | | 15A |
| PPY | PNP | Pancreatic polypeptide |
| PRDM13 | | PR domain containing 13 |
| PRDM16 | KIAA1675, MEL1, | PR domain containing 16 |
| | MGC166915, PFM13 | |
| PRDX2 | MGC4104, NKEFB, PRP, | Peroxiredoxin 2 |
| | PRX2, PRXII, TDPX1, TSA | |
| PRDX4 | AOE37-2 | Peroxiredoxin 4 |
| PRKCA | PKCA | Protein kinase C, alpha |
| PRKCB | PKCB, PRKCB1, PRKCB2 | Protein kinase C, beta |
| PRKCE | | Protein kinase C, epsilon |
| PRKCH | PKC-L, PKCL, PRKCL | Protein kinase C, eta |
| PRKCI | DXS1179E, PKCI | Protein kinase C, iota |
| PRKCQ | | Protein kinase C, theta |
| PRKDC | DNA-PKcs, DNAPK, DNPK1, | Protein kinase, DNA-activated, catalytic |
| | HYRC, HYRC1, p350, XRCC7 | polypeptide |
| PRL | | Prolactin |
| PROC | | Protein C (inactivator of coagulation |
| | | factors Va and VIIIa) |
| PRSS1 | TRY1 | Protease, serine, 1 (trypsin 1) |
| PSCA | | Prostate stem cell antigen |
| PSMD4 | AF, AF-1, Rpn10, S5A | Proteasome (prosome, macropain) 26S |
| | | subunit, non-ATPase, 4 |
| PTCH1 | BCNS, NBCCS, PTCH | Patched 1 |
| PTCH2 | | Patched 2 |
| PTGS1 | COX1, PGHS-1, PTGHS | Prostaglandin-endoperoxide synthase 1 |
| | | (prostaglandin G/H synthase and |
| | | cyclooxygenase) |
| PTGS2 | COX2 | Prostaglandin-endoperoxide synthase 2 |
| | | (prostaglandin G/H synthase and |
| | | cyclooxygenase) |
| PTH | PTH1 | Parathyroid hormone |
| PTHLH | HHM, PLP, PTHR, PTHRP | Parathyroid hormone-like hormone |
| PTK2 | FADK, FAK, FAK1, PPP1R71 | Protein tyrosine kinase 2 |
| PTN | HBGF8, HBNF, NEGF1 | Pleiotrophin |
| PTPRO | GLEPP1, NPHS6, PTP-oc, PTP- | Protein tyrosine phosphatase, receptor |
| | U2, PTPU2 | type, O |
| PTTG1 | EAP1, HPTTG, PTTG, securin, | Pituitary tumor-transforming 1 |
| | TUTR1 | |
| PURA | PUR-ALPHA, PUR1, | Purine-rich element binding protein A |
| | PURALPHA | |
| PZP | CPAMD6 | Pregnancy-zone protein |
| RAB11FIP3 | eferin, KIAA0665, Rab11-FIP3 | RAB11 family interacting protein 3 (class II) |
| RAB18 | | RAB18, member RAS oncogene family |
| RAB25 | CATX-8 | RAB25, member RAS oncogene family |
| RAC1 | p21-Rac1, Rac-1, TC-25 | Ras-related C3 botulinum toxin substrate 1 |
| | | (rho family, small GTP binding protein |
| | | Rac1) |
| RAD23A | HHR23A, MGC111083 | RAD23 homolog A (S. cerevisiae) |
| RAD23B | HHR23B, HR23B, P58 | RAD23 homolog B (S. cerevisiae) |
| RAD51 | BRCC5, HsRad51, HsT16930, | RAD51 recombinase |
| | RAD51A, RECA | |
| RAD51D | HsTRAD, R51H3, RAD51L3, | RAD51 paralog D |
| | Trad | |
| RAD52 | | RAD52 homolog (S. cerevisiae) |
| RAD54B | RDH54 | RAD54 homolog B (S. cerevisiae) |
| RAF1 | c-Raf, CRAF, Raf-1 | Raf-1 proto-oncogene, serine/threonine |
| | | kinase |
| RARA | NR1B1, RAR | Retinoic acid receptor, alpha |
| RARB | HAP, NR1B2, RRB2 | Retinoic acid receptor, beta |
| RARG | NR1B3, RARC | Retinoic acid receptor, gamma |
| RASA1 | CM-AVM, GAP, p120GAP, | RAS p21 protein activator (GTPase |
| | p120RASGAP, RASA | activating protein) 1 |
| RB1 | OSRC, PPP1R130, RB | Retinoblastoma 1 |
| RBBP4 | lin-53, NURF55, RbAp48 | Retinoblastoma binding protein 4 |
| RBL1 | cp107, p107, PRB1 | Retinoblastoma-like 1 |
| RBL2 | p130, Rb2 | Retinoblastoma-like 2 |
| RBM6 | 3G2, DEF-3, DEF3, g16, NY- | RNA binding motif protein 6 |
| | LU-12 | |
| RBP4 | | Retinol binding protein 4, plasma |
| REL | c-Rel, I-Rel | V-rel avian reticuloendotheliosis viral |
| | | oncogene homolog |
| RELA | NFKB3, p65 | V-rel avian reticuloendotheliosis viral |
| | | oncogene homolog A |
| RELB | REL-B | V-rel avian reticuloendotheliosis viral |
| | | oncogene homolog B |
| RET | CDHF12, CDHR16, HSCR1, | Ret proto-oncogene |
| | MEN2A, MEN2B, MTC1, PTC, | |
| | RET51 | |
| RHOA | ARH12, ARHA, Rho12, RhoA, | Ras homolog family member A |
| | RHOH12 | |
| RHOB | ARH6, ARHB, MST081, RhoB, | Ras homolog family member B |
| | RHOH6 | |
| RHOC | ARH9, ARHC, RhoC | Ras homolog family member C |
| RPA2 | | Replication protein A2, 32 kDa |
| RPL27 | L27 | Ribosomal protein L27 |
| RPS3 | FLJ26283, FLJ27450, | Ribosomal protein S3 |
| | MGC87870, S3 | |
| RPS6KA1 | HU-1, RSK, RSK1 | Ribosomal protein S6 kinase, 90 kDa, |
| | | polypeptide 1 |
| RPS6KA3 | CLS, HU-3, MRX19, RSK, | Ribosomal protein S6 kinase, 90 kDa, |
| | RSK2 | polypeptide 3 |
| RXRA | NR2B1 | Retinoid X receptor, alpha |
| RXRB | H-2RIIBP, NR2B2, RCoR-1 | Retinoid X receptor, beta |
| RXRG | NR2B3 | Retinoid X receptor, gamma |
| S100A1 | S100-alpha, S100A | S100 calcium binding protein A1 |
| S100A2 | CAN19, S100L | S100 calcium binding protein A2 |
| S100A4 | 18A2, 42A, CAPL, FSP1, | S100 calcium binding protein A4 |
| | MTS1, P9KA, PEL98 | |
| S100A6 | 2A9, CABP, CACY, PRA | S100 calcium binding protein A6 |
| S100A7 | PSOR1, S100A7c | S100 calcium binding protein A7 |
| S100A8 | 60B8AG, CAGA, CFAG, | S100 calcium binding protein A8 |
| | CGLA, MRP8, P8 | |
| S100A9 | 60B8AG, CAGB, CFAG, | S100 calcium binding protein A9 |
| | CGLB, LIAG, MAC387, MIF, | |
| | MRP14, NIF, P14 | |
| S100B | S100beta | S100 calcium binding protein B |
| S1PR1 | CD363, D1S3362, edg-1, EDG1 | Sphingosine-1-phosphate receptor 1 |
| SAA1 | PIG4, SAA, TP53I4 | Serum amyloid A1 |
| SAA2 | | Serum amyloid A2 |
| SART1 | Ara1, SNRNP110, Snu66 | Squamous cell carcinoma antigen |
| | | recognized by T cells |
| SCGB1A1 | CC10, CC16, CCSP, UGB | Secretoglobin, family 1A, member 1 |
| | | (uteroglobin) |
| SCGB1D2 | LIPB, LPHB | Secretoglobin, family 1D, member 2 |
| SCGB2A1 | LPHC, MGB2, MGC71973, | Secretoglobin, family 2A, member 1 |
| | UGB3 | |
| SCGB2A2 | MGB1, MGC71974, UGB2 | Secretoglobin, family 2A, member 2 |
| SDC1 | CD138, SDC, SYND1, syndecan | Syndecan 1 |
| SELE | CD62E, ELAM, ELAM1, ESEL | Selectin E |
| SELL | CD62L, hLHRc, LAM-1, | Selectin L |
| | LAM1, Leu-8, LNHR, LSEL, | |
| | Lyam-1, LYAM1, PLNHR | |
| SELP | CD62, CD62P, GMP140, | Selectin P (granule membrane protein |
| | GRMP, PADGEM, PSEL | 140 kDa, antigen CD62) |
| SEMA3B | LUCA-1, SemA, sema5, | Sema domain, immunoglobulin domain |
| | SEMAA, semaV | (Ig), short basic domain, secreted, |
| | | (semaphorin) 3B |
| 2-Sep | DIFF6, hNedd5, KIAA0158, | Septin 2 |
| | NEDD5, Pnutl3 | |
| SERPINA1 | A1A, A1AT, AAT, alpha-1- | Serpin peptidase inhibitor, clade A (alpha-1 |
| | antitrypsin, alpha1AT, PI, PI1 | antiproteinase, antitrypsin), member 1 |
| SERPINA3 | AACT, ACT, alpha-1- | Serpin peptidase inhibitor, clade A (alpha-1 |
| | antichymotrypsin | antiproteinase, antitrypsin), member 3 |
| SERPINA5 | PAI3, PCI, PLANH3, PROCI | Serpin peptidase inhibitor, clade A (alpha-1 |
| | | antiproteinase, antitrypsin), member 5 |
| SERPINB2 | HsT1201, PAI2, PLANH2 | Serpin peptidase inhibitor, clade B |
| | | (ovalbumin), member 2 |
| SERPINB3 | HsT1196, SCC, SCCA1, T4-A | Serpin peptidase inhibitor, clade B |
| | | (ovalbumin), member 3 |
| SERPINB4 | LEUPIN, PI11, SCCA-2, | Serpin peptidase inhibitor, clade B |
| | SCCA1, SCCA2 | (ovalbumin), member 4 |
| SERPINE1 | PAI, PAI1, PLANH1 | Serpin peptidase inhibitor, clade E (nexin, |
| | | plasminogen activator inhibitor type 1), |
| | | member 1 |
| SERPINF1 | EPC-1, PEDF, PIG35 | Serpin peptidase inhibitor, clade F (alpha-2 |
| | | antiplasmin, pigment epithelium derived |
| | | factor), member 1 |
| SFN | YWHAS | Stratifin |
| SHBG | ABP, MGC126834, | Sex hormone-binding globulin |
| | MGC138391, TEBG | |
| SIRT2 | SIR2L | Sirtuin 2 |
| SKP2 | FBL1, FBXL1, p45 | S-phase kinase-associated protein 2, E3 |
| | | ubiquitin protein ligase |
| SLC19A1 | FOLT | Solute carrier family 19 (folate transporter), |
| | | member 1 |
| SLC2A1 | DYT18, GLUT, GLUT1, | Solute carrier family 2 (facilitated glucose |
| | HTLVR | transporter), member 1 |
| SLC3A2 | 4F2, 4F2HC, 4T2HC, CD98, | Solute carrier family 3 (amino acid |
| | CD98HC, MDU1, NACAE | transporter heavy chain), member 2 |
| SLPI | ALK1, ALP, BLPI, HUSI, | Secretory leukocyte peptidase inhibitor |
| | HUSI-I, WAP4, WFDC4 | |
| SMAD1 | JV4-1, MADH1, MADR1 | SMAD family member 1 |
| SMAD2 | JV18-1, MADH2, MADR2 | SMAD family member 2 |
| SMAD3 | HsT17436, JV15-2, MADH3 | SMAD family member 3 |
| SMAD4 | DPC4, MADH4 | SMAD family member 4 |
| SMYD3 | KMT3E, ZMYND1, ZNFN3A1 | SET and MYND domain containing 3 |
| SOD1 | ALS, ALS1, IPOA | Superoxide dismutase 1, soluble |
| SOD2 | | Superoxide dismutase 2, mitochondrial |
| SOX1 | | SRY (sex determining region Y)-box 1 |
| SOX9 | CMD1, CMPD1, SRA1 | SRY (sex determining region Y)-box 9 |
| SP1 | | Sp1 transcription factor |
| SPARC | ON | Secreted protein, acidic, cysteine-rich |
| | | (osteonectin) |
| SPARCL1 | MAST9 | SPARC-like 1 (hevin) |
| SPINK1 | PCTT, PSTI, Spink3, TATI | Serine peptidase inhibitor, Kazal type 1 |
| SPINT1 | HAI, MANSC2 | Serine peptidase inhibitor, Kunitz type 1 |
| SPINT2 | HAI-2, Kop | Serine peptidase inhibitor, Kunitz type, 2 |
| SPP1 | BNSP, BSPI, ETA-1, OPN | Secreted phosphoprotein 1 |
| SPRR1B | GADD33, SPRR1 | Small proline-rich protein 1B |
| SPRR3 | | Small proline-rich protein 3 |
| SPRY1 | hSPRY1 | Sprouty homolog 1, antagonist of FGF |
| | | signaling (Drosophila) |
| SRC | ASV, c-src, SRC1 | SRC proto-oncogene, non-receptor tyrosine |
| | | kinase |
| SRD5A1 | | Steroid-5-alpha-reductase, alpha |
| | | polypeptide 1 (3-oxo-5 alpha-steroid delta |
| | | 4-dehydrogenase alpha 1) |
| SRD5A2 | | Steroid-5-alpha-reductase, alpha |
| | | polypeptide 2 (3-oxo-5 alpha-steroid delta |
| | | 4-dehydrogenase alpha 2) |
| SST | SMST | Somatostatin |
| SSX2 | CT5.2a, HD21, HOM-MEL-40, | Synovial sarcoma, X breakpoint 2 |
| | MGC119055, MGC15364, | |
| | MGC3884, SSX | |
| SSX2B | CT5.2b | Synovial sarcoma, X breakpoint 2B |
| ST14 | HAI, MT-SP1, PRSS14, SNC19, | Suppression of tumorigenicity 14 (colon |
| | TMPRSS14 | carcinoma) |
| STARD3 | es64, MLN64 | StAR-related lipid transfer (START) |
| | | domain containing 3 |
| STAT4 | | Signal transducer and activator of |
| | | transcription 4 |
| STAT5A | MGF, STAT5 | Signal transducer and activator of |
| | | transcription 5A |
| STEAP1 | PRSS24, STEAP | Six transmembrane epithelial antigen of the |
| | | prostate 1 |
| STMN1 | C1orf215, FLJ32206, Lag, | Stathmin 1 |
| | LAP18, OP18, PP17, PP19, | |
| | PR22, SMN | |
| STRAP | MAWD, pt-wd, UNRIP | Serine/threonine kinase receptor associated |
| | | protein |
| STT3A | ITM1, MGC9042, STT3-A, | STT3A, subunit of the |
| | TMC | oligosaccharyltransferase complex |
| | | (catalytic) |
| SULT1E1 | EST, STE | Sulfotransferase family 1E, estrogen- |
| | | preferring, member 1 |
| TAGLN | DKFZp686P11128, SM22, | Transgelin |
| | SMCC, TAGLN1, WS3-10 | |
| TDRD6 | bA446F17.4, CT41.2, NY-CO- | Tudor domain containing 6 |
| | 45, SPATA36 | |
| TEK | CD202b, TIE-2, TIE2, VMCM, | TEK tyrosine kinase, endothelial |
| | VMCM1 | |
| TERT | EST2, hEST2, TCS1, TP2, TRT | Telomerase reverse transcriptase |
| TF | PRO1557, PRO2086 | Transferrin |
| TFAP2B | AP2-B | Transcription factor AP-2 beta (activating |
| | | enhancer binding protein 2 beta) |
| TFDP1 | Dp-1, DP1, DRTF1 | Transcription factor Dp-1 |
| TFDP2 | Dp-2 | Transcription factor Dp-2 (E2F |
| | | dimerization partner 2) |
| TFF1 | BCEI, D21S21, HP1.A, HPS2, | Trefoil factor 1 |
| | pNR-2, pS2 | |
| TFF2 | SML1 | Trefoil factor 2 |
| TFF3 | HITF, ITF | Trefoil factor 3 (intestinal) |
| TFRC | CD71, p90, TFR1 | Transferrin receptor |
| TG | AITD3, TGN | Thyroglobulin |
| TGFA | | Transforming growth factor, alpha |
| TGFB1 | CED, DPD1, TGFB, TGFbeta | Transforming growth factor, beta 1 |
| TGFB2 | | Transforming growth factor, beta 2 |
| TGFB3 | ARVD, ARVD1 | Transforming growth factor, beta 3 |
| TGFBR3 | betaglycan, BGCAN | Transforming growth factor, beta receptor |
| | | III |
| TGM4 | TGP | Transglutaminase 4 |
| TGM7 | TGMZ | Transglutaminase 7 |
| THBS1 | THBS, THBS-1, TSP, TSP-1, | Thrombospondin 1 |
| | TSP1 | |
| THBS2 | TSP2 | Thrombospondin 2 |
| THBS4 | | Thrombospondin 4 |
| THPO | MGDF, MPLLG, TPO | Thrombopoietin |
| THRA | AR7, EAR-7.1/EAR-7.2, ERBA, | Thyroid hormone receptor, alpha |
| | ERBA1, NR1A1, THRA1, | |
| | THRA2, THRA3 | |
| THRB | ERBA-BETA, ERBA2, GRTH, | Thyroid hormone receptor, beta |
| | NR1A2, PRTH, THR1, THRB1, | |
| | THRB2 | |
| TIE1 | JTK14, TIE | Tyrosine kinase with immunoglobulin-like |
| | | and EGF-like domains 1 |
| TIMP1 | CLGI, EPO, TIMP | TIMP metallopeptidase inhibitor 1 |
| TIMP2 | CSC-21K | TIMP metallopeptidase inhibitor 2 |
| TIMP3 | SFD | TIMP metallopeptidase inhibitor 3 |
| TK1 | | Thymidine kinase 1, soluble |
| TMF1 | ARA160, TMF | TATA element modulatory factor 1 |
| TMPRSS2 | PRSS10 | Transmembrane protease, serine 2 |
| TMPRSS3 | DFNB10, DFNB8 | Transmembrane protease, serine 3 |
| TNC | DFNA56, HXB, MGC167029, | Tenascin C |
| | TN | |
| TNF | DIF, TNF-alpha, TNFA, | Tumor necrosis factor |
| | TNFSF2 | |
| TNFAIP2 | B94, EXOC3L3 | Tumor necrosis factor, alpha-induced |
| | | protein 2 |
| TNFAIP3 | A20, OTUD7C | Tumor necrosis factor, alpha-induced |
| | | protein 3 |
| TNFRSF10A | Apo2, CD261, DR4, TRAILR-1 | Tumor necrosis factor receptor |
| | | superfamily, member 10a |
| TNFRSF10B | CD262, DR5, KILLER, TRAIL- | Tumor necrosis factor receptor |
| | R2, TRICK2A, TRICKB | superfamily, member 10b |
| TNFRSF10C | CD263, DcR1, LIT, TRAILR3, | Tumor necrosis factor receptor |
| | TRID | superfamily, member 10c, decoy without |
| | | an intracellular domain |
| TNFRSF10D | CD264, DcR2, TRAILR4, | Tumor necrosis factor receptor |
| | TRUNDD | superfamily, member 10d, decoy with |
| | | truncated death domain |
| TNFRSF11B | OCIF, OPG, TR1 | Tumor necrosis factor receptor |
| | | superfamily, member 11b |
| TNFRSF12A | CD266, FN14, TweakR | Tumor necrosis factor receptor |
| | | superfamily, member 12A |
| TNFRSF14 | ATAR, CD270, HVEA, HVEM, | Tumor necrosis factor receptor |
| | LIGHTR, TR2 | superfamily, member 14 |
| TNFRSF1A | CD120a, TNF-R, TNF-R-I, | Tumor necrosis factor receptor |
| | TNF-R55, TNFAR, TNFR1, | superfamily, member 1A |
| | TNFR60 | |
| TNFRSF1B | CD120b, p75, TNF-R-II, TNF- | Tumor necrosis factor receptor |
| | R75, TNFBR, TNFR2, TNFR80 | superfamily, member 1B |
| TNFRSF4 | ACT35, CD134, OX40, | Tumor necrosis factor receptor |
| | TXGP1L | superfamily, member 4 |
| TNFRSF8 | CD30, D1S166E, KI-1 | Tumor necrosis factor receptor |
| | | superfamily, member 8 |
| TNFRSF9 | 4-1BB, CD137, ILA | Tumor necrosis factor receptor |
| | | superfamily, member 9 |
| TNFSF10 | Apo-2L, CD253, TL2, TRAIL | Tumor necrosis factor (ligand) superfamily, |
| | | member 10 |
| TNFSF11 | CD254, ODF, OPGL, RANKL, | Tumor necrosis factor (ligand) superfamily, |
| | TRANCE | member 11 |
| TNFSF13 | APRIL, CD256 | Tumor necrosis factor (ligand) superfamily, |
| | | member 13 |
| TNFSF13B | BAFF, BLYS, CD257, TALL-1, | Tumor necrosis factor (ligand) superfamily, |
| | TALL1, THANK, TNFSF20 | member 13b |
| TNFSF4 | CD252, gp34, OX-40L, TXGP1 | Tumor necrosis factor (ligand) superfamily, |
| | | member 4 |
| TNFSF8 | CD153, CD30LG | Tumor necrosis factor (ligand) superfamily, |
| | | member 8 |
| TNK2 | ACK, ACK1, p21cdc42Hs | Tyrosine kinase, non-receptor, 2 |
| TOP2A | TOP2 | Topoisomerase (DNA) II alpha 170 kDa |
| TP53 | LFS1, p53 | Tumor protein p53 |
| TP53BP2 | 53BP2, ASPP2, PPP1R13A | Tumor protein p53 binding protein 2 |
| TPD52 | D52, hD52, N8L | Tumor protein D52 |
| TPI1 | | Triosephosphate isomerase 1 |
| TPM1 | C15orf13, CMH3 | Tropomyosin 1 (alpha) |
| TPM2 | AMCD1, DA1, NEM4 | Tropomyosin 2 (beta) |
| TPX2 | C20orf1, C20orf2, DIL-2, p100 | TPX2, microtubule-associated |
| TRAF1 | EBI6 | TNF receptor-associated factor 1 |
| TRAF2 | TRAP3 | TNF receptor-associated factor 2 |
| TRAF4 | CART1, MLN62, RNF83 | TNF receptor-associated factor 4 |
| TRIM25 | EFP, RNF147, ZNF147 | Tripartite motif containing 25 |
| TRIP4 | HsT17391, ZC2HC5 | Thyroid hormone receptor interactor 4 |
| TRO | KIAA1114, MAGE-D3, | Trophinin |
| | MAGED3 | |
| TSG101 | TSG10, VPS23 | Tumor susceptibility 101 |
| TSPAN8 | CO-029, TM4SF3 | Tetraspanin 8 |
| TSPO | BZRP, DBI, IBP, MBR, mDRC, | Translocator protein (18 kDa) |
| | PBR, pk18, PKBS | |
| TTR | CTS, CTS1, HsT2651, PALB | Transthyretin |
| TUSC2 | C3orf11, FUS1, PAP, PDAP2 | Tumor suppressor candidate 2 |
| TWIST1 | ACS3, bHLHa38, BPES2, | Twist family bHLH transcription factor 1 |
| | BPES3, CRS, CRS1, H-twist, | |
| | SCS, TWIST | |
| TXLNA | DKFZp451J0118 | Taxilin alpha |
| TYMP | ECGF1, MNGIE | Thymidine phosphorylase |
| TYMS | HsT422, TMS, TS, Tsase | Thymidylate synthetase |
| TYRO3 | Brt, Dtk, RSE, Sky, Tif | TYRO3 protein tyrosine kinase |
| UBA1 | A1S9T, CFAP124, GXP1, | Ubiquitin-like modifier activating enzyme 1 |
| | POC20, UBE1, UBE1X | |
| UBE2C | UBCH10 | Ubiquitin-conjugating enzyme E2C |
| UBE2I | UBC9 | Ubiquitin-conjugating enzyme E2I |
| UBE2N | MGC8489, UBC13, UbcH-ben | Ubiquitin-conjugating enzyme E2N |
| UGT1A10 | UGT1J | UDP glucuronosyltransferase 1 family, |
| | | polypeptide A10 |
| UGT1A3 | UGT1C | UDP glucuronosyltransferase 1 family, |
| | | polypeptide A3 |
| UGT1A4 | HUG-BR2, UGT1D | UDP glucuronosyltransferase 1 family, |
| | | polypeptide A4 |
| UGT1A8 | UGT1H | UDP glucuronosyltransferase 1 family, |
| | | polypeptide A8 |
| UGT1A9 | HLUGP4, LUGP4, UGT1AI | UDP glucuronosyltransferase 1 family, |
| | | polypeptide A9 |
| USH1C | AIE-75, DFNB18, harmonin, | Usher syndrome 1C (autosomal recessive, |
| | NY-CO-37, NY-CO-38, PDZ- | severe) |
| | 73, PDZ73, PDZD7C | |
| VAMP3 | CEB | Vesicle-associated membrane protein 3 |
| VCAM1 | CD106 | Vascular cell adhesion molecule 1 |
| VEGFA | VEGF, VEGF-A, VPF | Vascular endothelial growth factor A |
| VEGFB | VEGFL, VRF | Vascular endothelial growth factor B |
| VEGFC | VRP | Vascular endothelial growth factor C |
| VHL | VHL1 | Von Hippel-Lindau tumor suppressor, E3 |
| | | ubiquitin protein ligase |
| VIL1 | D2S1471, VIL | Villin 1 |
| VIP | | Vasoactive intestinal peptide |
| VTN | VN | Vitronectin |
| VWF | F8VWF | Von Willebrand factor |
| WEE1 | | WEE1 G2 checkpoint kinase |
| WFDC2 | dJ461P17.6, EDDM4, HE4, | WAP four-disulfide core domain 2 |
| | WAP5 | |
| WISP1 | CCN4 | WNT1 inducible signaling pathway protein 1 |
| WNT1 | INT1 | Wingless-type MMTV integration site |
| | | family, member 1 |
| WNT2 | INT1L1, IRP | Wingless-type MMTV integration site |
| | | family member 2 |
| WRN | RECQ3, RECQL2 | Werner syndrome, RecQ helicase-like |
| WT1 | AWT1, GUD, WAGR, WIT-2 | Wilms tumor 1 |
| XBP1 | XBP2 | X-box binding protein 1 |
| XIAP | API3, BIRC4, hILP | X-linked inhibitor of apoptosis |
| XPA | XP1, XPAC | Xeroderma pigmentosum, |
| | | complementation group A |
| XPC | RAD4, XPCC | Xeroderma pigmentosum, |
| | | complementation group C |
| XRCC2 | | X-ray repair complementing defective |
| | | repair in Chinese hamster cells 2 |
| XRCC3 | | X-ray repair complementing defective |
| | | repair in Chinese hamster cells 3 |
| XRCC4 | | X-ray repair complementing defective |
| | | repair in Chinese hamster cells 4 |
| XRCC5 | KARP-1, KU80, Ku86, KUB2 | X-ray repair complementing defective |
| | | repair in Chinese hamster cells 5 (double- |
| | | strand-break rejoining) |
| XRCC6 | D22S671, D22S731, G22P1, | X-ray repair complementing defective |
| | KU70, ML8 | repair in Chinese hamster cells 6 |
| YBX1 | BP-8, CSDA2, CSDB, DBPB, | Y box binding protein 1 |
| | MDR-NF1, NSEP-1, NSEP1, | |
| | YB-1, YB1 | |
| YWHAB | YWHAA | Tyrosine 3-monooxygenase/tryptophan 5- |
| | | monooxygenase activation protein, beta |
| YWHAE | FLJ45465 | Tyrosine 3-monooxygenase/tryptophan 5- |
| | | monooxygenase activation protein, epsilon |
| YWHAH | YWHA1 | Tyrosine 3-monooxygenase/tryptophan 5- |
| | | monooxygenase activation protein, eta |
| ZBTB16 | PLZF, ZNF145 | Zinc finger and BTB domain containing 16 |
| ZMAT3 | FLJ12296, MGC10613, | Zinc finger, matrin-type 3 |
| | PAG608, WIG-1, WIG1 | |

TABLE 2

| Gene | Gene synonym | Gene description |
|---|---|---|
| HIF1A | HIF-1α | hypoxia-inducible factor 1, alpha subunit |
| ARNT | HIF-1β | aryl hydrocarbon receptor nuclear translocator |
| EPAS1 | HIF-2α | endothelial PAS domain protein 1 |
| ARNT2 | HIF-2β | aryl-hydrocarbon receptor nuclear translocator 2 |
| HIF3A | HIF-3α | hypoxia inducible factor 3, alpha subunit |
| ARNTL | HIF-3β | aryl-hydrocarbon receptor nuclear translocator 3 |
| CA9 | CA IX | Carbonic anhydrase 9 |
| SLC2A1 | GLUT-1, GLUT | Solute Carrier Family 2 Member 1 |

In one embodiment, the biomarker is a molecular marker for tumor hypoxia. In one embodiment, the molecular marker for tumor hypoxia is a hypoxia-inducible factor (HIF). In one embodiment, the measurable aspect of HIF is its expression status. In one embodiment, the biomarker is overexpression of HIF.

Thus, in certain aspects of the disclosure, the biomarker is HIF-1α which is differentially present in a subject of one phenotypic status, e.g., a patient having cancer, e.g., colon cancer, breast cancer, pancreatic cancer, kidney cancer, prostate cancer, brain cancer, bladder cancer, cervical cancer, non-small-cell lung carcinoma, oligodendroglioma, oropharyngeal cancer, ovarian cancer, endometrial cancer, esophageal cancer, head and neck cancer, and stomach cancer, as compared with another phenotypic status, e.g., a normal undiseased subject or a patient having cancer without overexpression HIF-1α. In one embodiment, the biomarker is overexpression of HIF-1α.

Biomarker standards can be predetermined, determined concurrently, or determined after a biological sample is obtained from the subject. Biomarker standards for use with the methods described herein can, for example, include data from samples from subjects without cancer; data from samples from subjects with cancer, e.g., breast cancer, that is not metastatic; and data from samples from subjects with cancer, e.g., breast cancer, that metastatic. Comparisons can be made to establish predetermined threshold biomarker standards for different classes of subjects, e.g., diseased vs. non-diseased subjects. The standards can be run in the same assay or can be known standards from a previous assay.

A biomarker is differentially present between different phenotypic status groups if the mean or median expression or mutation levels of the biomarker is calculated to be different, i.e., higher or lower, between the groups. Thus, biomarkers provide an indication that a subject, e.g., a cancer patient, belongs to one phenotypic status or another.

In addition to individual biological compounds, e.g., HIF-1α or HIF-2α, the term “biomarker” as used herein is meant to include groups, sets, or arrays of multiple biological compounds. For example, the combination of HIF-1α and HIF-1α may comprise a biomarker. The term “biomarker” may comprise one, two, three, four, five, six, seven, eight, nine, ten, fifteen, twenty, twenty five, thirty, or more, biological compounds.

The determination of the expression level or mutation status of a biomarker in a patient can be performed using any of the many methods known in the art. Any method known in the art for quantitating specific proteins and/or detecting HIF expression, or the expression or mutation levels of any other biomarker in a patient or a biological sample may be used in the methods of the disclosure. Examples include, but are not limited to, PCR (polymerase chain reaction), or RT-PCR, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998). Certain embodiments of the disclosure include methods wherein biomarker RNA expression (transcription) is determined. Other embodiments of the disclosure include methods wherein protein expression in the biological sample is determined. See, for example, Harlow et al., Antibodies: A Laboratory Manual, Cold Spring Harbor Laboratory, Cold Spring Harbor, NY, (1988) and Ausubel et al., Current Protocols in Molecular Biology, John Wiley & Sons, New York 3rd Edition, (1995). For northern blot or RT-PCR analysis, RNA is isolated from the tumor tissue sample using RNAse free techniques. Such techniques are commonly known in the art.

In one embodiment of the disclosure, a biological sample is obtained from the patient and cells in the biopsy are assayed for determination of biomarker expression or mutation status.

In one embodiment of the disclosure, PET imaging is used to determine biomarker expression.

In another embodiment of the disclosure, Northern blot analysis of biomarker transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabled probes and their use are well known in the art. The presence and or relative levels of expression of the biomarker being assayed can be quantified using, for example, densitometry.

In another embodiment of the disclosure, biomarker expression and/or mutation status is determined using RT-PCR. RT-PCR allows detection of the progress of a PCR amplification of a target gene in real time. Design of the primers and probes required to detect expression and/or mutation status of a biomarker of the disclosure is within the skill of a practitioner of ordinary skill in the art. RT-PCR can be used to determine the level of RNA encoding a biomarker of the disclosure in a tumor tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5, end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In another embodiment of the disclosure, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In another embodiment of the disclosure, the expression of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In another embodiment of the disclosure, the expression of a biomarker is evaluated by use of a gene chip or microarray. Such techniques are within ordinary skill held in the art.

### VI. Definitions

The vinca alkaloid N-oxides of the present disclosure may exist as pharmaceutically acceptable salts. Nonlimiting examples of salts of vinca alkaloid N-oxides include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts.

The term “biological sample” as used herein refers any tissue or fluid from a patient that is suitable for detecting a biomarker, such as HIF-1α expression status. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarker expression and/or mutation using any technique known in the art and can be obtained using techniques that are well within the scope of ordinary knowledge of a clinical practioner. In one embodiment of the disclosure, the biological sample comprises blood cells.

The term hypoxia-inducible factor or “HIF” as used herein refers to proteins that sense and respond to oxygen deficiency by acting as transcription factors. The HIF signaling cascade mediates the effects of hypoxia, the state of low oxygen concentration, on the cell. Wilkins et al., ChemMedChem 11:773-786 (2016). The following are memberes of the human HIF family:

| member | gene | protein |
|---|---|---|
| HIF-1α | HIF1A | hypoxia-inducible factor 1, alpha subunit |
| HIF-1β | ARNT | aryl hydrocarbon receptor nuclear translocator |
| HIF-2α | EPAS1 | endothelial PAS domain protein 1 |
| HIF-2β | ARNT2 | aryl-hydrocarbon receptor nuclear translocator 2 |
| HIF-3α | HIF3A | hypoxia inducible factor 3, alpha subunit |
| HIF-3β | ARNTL | aryl-hydrocarbon receptor nuclear translocator 3 |

HIF proteins are overexpressed in many human cancers. Zhong et al., Cancer Research 59:5830-5835 (1999). Talks et al., The American Journal of Pathology. 157:411-21 (2000). Wigerup et al., Pharmacology & Therapeutics 164:152-169 (2016). HIF overexpression is implicated in promoting tumor growth and metastasis through its role in initiating angiogenesis and regulating cellular metabolism to overcome hypoxia. Hypoxia promotes apoptosis in both normal and tumor cells. But hypoxic conditions in cancer tumors, along with accumulation of genetic alternations, often contribute to HIF overexpression. Semenza, Nature Reviews. Cancer 3:721-32 (2003).

Significant HIF expression has been noted in most solid tumors including cancers of the colon, breast, pancreas, kidneys, prostate, ovary, brain, and bladder. Clinically, elevated HIF levels in a number of cancers, including cervical cancer, non-small-cell lung carcinoma, breast cancer (LV-positive and negative), oligodendroglioma, oropharyngeal cancer, ovarian cancer, endometrial cancer, esophageal cancer, head and neck cancer, and stomach cancer, have been associated with aggressive tumor progression, and thus has been implicated as a predictive and prognostic marker for resistance to radiation treatment, chemotherapy, and increased mortality.

HIFIA (or HIF-1α) expression may also regulate breast tumor progression. Bos et al., Journal of the National Cancer Institute 93:309-14 (2001). Elevated HIFIA levels may be detected in early cancer development, and have been found in early ductal carcinoma in situ, a pre-invasive stage in breast cancer development, and is also associated with increased microvasculature density in tumor lesions. Moreover, despite histologically-determined low-grade, lymph-node negative breast tumor in a subset of patients examined, detection of significant HIF1A expression was able to independently predict poor response to therapy. Bos et al., Cancer 97:1573-81 (2003). Similar findings have been reported in brain cancer and ovarian cancer studies as well, and suggest at regulatory role of HIF-1α in initiating angiogenesis through interactions with pro-angiogenic factors such as VEGF. Studies of glioblastoma multiforme show striking similarity between HIF1A expression pattern and that of VEGF gene transcription level. In addition, high-grade glioblastoma multiform tumors with high VEGF expression pattern, similar to breast cancer with HIF1A overexpression, display significant signs of tumor neovascularization. This further suggests the regulatory role of HIF-1α in promoting tumor progression, likely through hypoxia-induced VEGF expression pathways. Powis and Kirkpatrick, Molecular Cancer Therapeutics 3:647-54 (2004).

HIF1A overexpression in tumors may also occur in a hypoxia-independent pathway. In hemagioblastoma, HIF1A expression is found in most cells sampled from the well-vascularized tumor. Although in both renal carcinoma and hemagioblastoma, the von Hippel-Lindau gene is inactivated, HIF1A is still expressed at high levels. In addition to VEGF overexpression in response elevated HIF1A levels, the PI3K/AKT pathway is also involved in tumor growth. In prostate cancers, the commonly occurring PTEN mutation is associated with tumor progression toward aggressive stage, increased vascular density and angiogenesis.

During hypoxia, tumor suppressor p53 overexpression may be associated with HIF1A-dependent pathway to initiate apoptosis. Moreover, p53-independent pathway may also induce apoptosis through the Bcl-2 pathway. However, overexpression of HIF1A is cancer- and individual-specific, and depends on the accompanying genetic alternations and levels of pro- and anti-apoptotic factors present. One study on epithelial ovarian cancer shows HIF1A and nonfunctional tumor suppressor p53 is correlated with low levels of tumor cell apoptosis and poor prognosis. Further, early-stage esophageal cancer patients with demonstrated overexpression of HIF1 and absence of BCL2 expression also failed photodynamic therapy. Studies of glioblastoma multiforme show striking similarity between HIF1A protein expression pattern and that of VEGF gene transcription level.

The term “liposome” refers to microscopic lipid vesicles composed of a bilayer of phospholipids or any similar amphipathic lipids encapsulating an internal aqueous medium. Bozzuto and Molinari, International Journal of Nanomedicine 10:975-999 (2015). Liposomes of the present disclosure can be unilamellar vesicles such as small unilamellar vesicles (SUVs) and large unilamellar vesicles (LUVs), and smaller multilamellar vesicles (MLV), typically varying in size, e.g., from 50 nm to 500 nm. No particular limitation is imposed on the liposomal membrane structure in the present disclosure. The term liposomal membrane refers to the bilayer of phospholipids separating the internal aqueous medium from the external aqueous medium.

Exemplary liposomal membranes useful in the current disclosure may be formed from a variety of vesicle-forming lipids, typically including dialiphatic chain lipids, such as phospholipids, diglycerides, dialiphatic glycolipids, egg sphingomyelin and glycosphingolipid, cholesterol, and derivatives thereof, and combinations thereof. Phospholipids are amphiphilic agents having hydrophobic groups formed of long-chain alkyl chains, and a hydrophilic group containing a phosphate moiety. The group of phospholipids includes phosphatidic acid, phosphatidyl glycerols, phosphatidylcholines, phosphatidylethanolamines, phosphatidylinositols, phosphatidylserines, and mixtures thereof. In some embodiments, the phospholipids are chosen from egg yolk phosphatidylcholine (EYPC), soy phosphatidylcholine (SPC), palmitoyl-oleoyl phosphatidylcholine, dioleyl phosphatidylcholine, 1,2-dipalmitoyl-sn-glycero-3-phosphocholine (DPPC), 1,2-dimyristoyl-sn-phosphatidylcholine (DMPC), hydrogenated soy phosphatidylcholine (HSPC), distearoyl phosphatidylcholine (DSPC), or hydrogenated egg yolk phosphatidylcholine (HEPC), egg phosphatidylglycerol, distearoylphosphatidylglycerol (DSPG), sterol modified lipids, cationic lipids and zwitterlipids.

Liposomes can be prepared by any of the techniques known in the art. See, e.g., Shah et al., Journal of Controlled Release 253:37-45 (2017). For example, the liposomes can be formed by the conventional technique for preparing multilamellar lipid vesicles (MLVs), that is, by depositing one or more selected lipids on the inside walls of a suitable vessel by dissolving the lipids in chloroform and then evaporating the chloroform, and by then adding the aqueous solution which is to be encapsulated to the vessel, allowing the aqueous solution to hydrate the lipid, and swirling or vortexing the resulting lipid suspension. This process engenders a mixture including the desired liposomes. Alternatively, techniques used for producing large unilamellar lipid vesicles (LUVs), such as reverse-phase evaporation, infusion procedures, and detergent dilution, can be used to produce the liposomes. A review of these and other methods for producing lipid vesicles can be found in: Liposome Technology: Liposome preparation and related Techniques, 3^{rd} addition, 2006, G. Gregoriadis, ed.). For example, the lipid-containing particles can be in the form of steroidal lipid vesicles, stable plurilamellar lipid vesicles (SPLVs), monophasic vesicles (MPVs), or lipid matrix carriers (LMCs). In the case of MLVs, if desired, the liposomes can be subjected to multiple (five or more) freeze-thaw cycles to enhance their trapped volumes and trapping efficiencies and to provide a more uniform interlamellar distribution of solute.

Following liposome preparation, the liposomes are optionally sized to achieve a desired size range and relatively narrow distribution of liposome sizes. A size range of from about 30 to about 200 nanometers allows the liposome suspension to be sterilized by filtration through a conventional sterile filter, typically a 0.22 micron or 0.4 micron filter. The filter sterilization method can be carried out on a high throughput basis if the liposomes have been sized down to about 20-300 nanometers. Several techniques are available for sizing liposomes to a desired size. Sonicating a liposome suspension either by bath or probe sonication produces a progressive size reduction down to small unilamellar vesicles less than about 50 nanometer in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 50 and 500 nanometers, are observed. In both methods, the particle size distribution can be monitored by conventional laser-beam particle size determination. Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. Other useful sizing methods such as sonication, solvent vaporization or reverse phase evaporation are known to those of skill in the art.

Exemplary liposomes for use in various embodiments of the disclosure have a size of from about 30 nm to about 300 nm, e.g., from about 50 nm to about 250 nm.

The internal aqueous medium, as referred to herein, typically is the original medium in which the liposomes were prepared and which initially becomes encapsulated upon formation of the liposome. In accordance with the present disclosure, freshly prepared liposomes encapsulating the original aqueous medium can be used directly for active loading. Embodiments are also envisaged however wherein the liposomes, after preparation, are dehydrated, e.g. for storage. In such embodiments the present process may involve addition of the dehydrated liposomes directly to the external aqueous medium used to create the transmembrane gradients. However it is also possible to hydrate the liposomes in another external medium first, as will be understood by those skilled in the art. Liposomes are optionally dehydrated under reduced pressure using standard freeze-drying equipment or equivalent apparatus. In various embodiments, the liposomes and their surrounding medium are frozen in liquid nitrogen before being dehydrated and placed under reduced pressure. To ensure that the liposomes will survive the dehydration process without losing a substantial portion of their internal contents, one or more protective sugars are typically employed to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Typically, one or more sugars are included as part of either the internal or external media of the lipid vesicles. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the lipid vesicles during the liposome formation process. In addition to the sugars, a co-lyophilization agent such as glycine, betaine or carnitine, can be included to further increase the stability of the lyophilized liposome chelators. In these embodiments the external medium used during the active loading process should also preferably include one or more of the protective sugars.

As is generally known to those skilled in the art, polyethylene glycol (PEG)-lipid conjugates have been used extensively to improve circulation times for liposome-encapsulated functional compounds, to avoid or reduce premature leakage of the functional compound from the liposomal composition and to avoid detection of liposomes by the body's immune system. Attachment of PEG-derived lipids onto liposomes is called PEGylation. Hence, in one embodiment of the disclosure, the liposomes are PEGylated liposomes. Suitable PEG-derived lipids according to the disclosure, include conjugates of DSPE-PEG, functionalized with one of carboxylic acids, glutathione (GSH), maleimides (MAL), 3-(2-pyridyldithio) propionic acid (PDP), cyanur, azides, amines, biotin or folate, in which the molecular weight of PEG is between 2000 and 5000 g/mol. Other suitable PEG-derived lipids are mPEGs conjugated with ceramide, having either C₈- or C₁₆-tails, in which the molecular weight of mPEG is between 750 and 5000 daltons. Still other appropriate ligands are mPEGs or functionalized PEGs conjugated with glycerophospholipds like 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE) and 1,2-distearoyl-sn-glycero-3-phosphoethanolamine (DSPE), and the like. PEGylation of liposomes is a technique generally known by those skilled in the art.

In various embodiments, the liposomes are PEGylated with DSPE-mPEG conjugates (wherein the molecular weight of PEG is typically within the range of 750-5000 daltons, e.g. 2000 daltons). The phospholipid composition of an exemplary PEGylated lipsome of the disclosure may comprise up to, e.g., 0.8-20 mol % of PEG-lipid conjugates.

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. However, in one embodiment, administration of avinca alkaloid N-oxide and an immune checkpoint inhibitor leads to remission of the cancer.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase patient survival time by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, the vinca alkaloid N-oxide can be administered at the same time or sequentially in any order at different points in time as the immune checkpoint inhibitor. The vinca alkaloid N-oxide and the immune checkpoint inhibitor can be administered separately, in any appropriate form and by any suitable route, e.g., by IV injection. When the vinca alkaloid N-oxide and the immune checkpoint inhibitor are not administered concurrently, it is understood that they can be administered in any order to a patient in need thereof. For example, the vinca alkaloid N-oxide can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the immune checkpoint inhibitor. In various embodiments, vinca alkaloid N-oxide and the immune checkpoint inhibitor are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, no more than 48 hours apart, no more than 3 days apart, or no more than 1 week apart. In one embodiment, the vinca alkaloid N-oxide is administered 1-14 days prior to the day the immune checkpoint inhibitor is administered. In one embodiment, the vinca alkaloid N-oxide is administered 1-7 days prior to the day the immune checkpoint inhibitor is administered. In another embodiment, the vinca alkaloid N-oxide is also administered on the day the immune checkpoint inhibitor is administered.

### VII. Particular Embodiments

The disclosure provides the following particular embodiments.

Embodiment 1. A method of treating a patient having cancer, the method comprising administering to the patient in need thereof a therapeutically effective amount of:
(a) a vinca alkaloid N-oxide, or a pharmaceutically acceptable salt or solvate thereof; and
(b) an immune checkpoint inhibitor.

Embodiment 2. The method of Embodiment 1, wherein the vinca alkaloid N-oxide is a N_{b′}-oxide, or a pharmaceutically acceptable salt or solvate thereof.

Embodiment 3. The method of Embodiment 2, wherein the vinca alkaloid N-oxide is represented by a compound having Formula I:

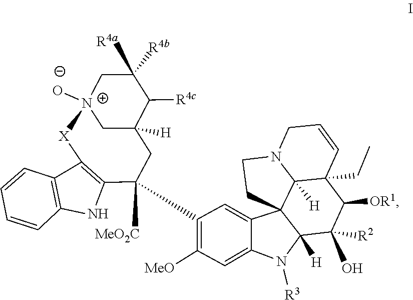

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 4. The method of Embodiment 3, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 5. The method of any one of Embodiments 1-4, wherein the vinca alkaloid N-oxide, or a pharmaceutically acceptable salt or solvate thereof, is administered to the patient encapsulated in a liposome.

Embodiment 6. The method of Embodiment 5, wherein the liposome comprises sphingomyelin and cholesterol.

Embodiment 7. The method of Embodiment 5, wherein the liposome comprises sphingomyelin, cholesterol, and 1,2-distearoyl-sn-glycero-3-phosphoethanolamine-N-[methoxy(polyethylene glycerol)-2000].

Embodiment 8. The method of any one of Embodiments 1-7, wherein immune checkpoint inhibitor is selected from the group consisting of a PD-1 inhibitor, a PD-L1 inhibitor, a CTLA-4 inhibitor, a LAG3 inhibitor, a TIGTI inhibitor, and a TIM3 inhibitor.

Embodiment 9. The method of Embodiment 8, wherein the immune checkpoint inhibitor is a PD-1 inhibitor.

Embodiment 10. The method of Embodiment 9, wherein the PD-1 inhibitor is an anti-PD-1 antibody.

Embodiment 11. The method of Embodiment 10, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 12. The method of Embodiment 8, wherein the immune checkpoint inhibitor is a PD-L1 inhibitor.

Embodiment 13. The method of Embodiment 12, wherein the PD-L1 inhibitor is an anti-PD-L1 antibody.

Embodiment 14. The method of Embodiment 13, wherein the anti-PD-L1 antibody is selected from the group consisting of avelumab, atezolizumab, durvalumab, and STI-A1014.

Embodiment 15. The method of Embodiment 8, wherein the immune checkpoint inhibitor is an anti-CTLA-4 inhibitor.

Embodiment 16. The method of Embodiment 15, wherein the anti-CTLA-4 inhibitor is an anti-CTLA-4 antibody.

Embodiment 17. The method of Embodiment 16, wherein the anti-CTLA-4 antibody is selected from the group consisting of ipilimumab and tremelimumab.

Embodiment 18. The method of Embodiment 8, wherein the immune checkpoint inhibitor is a LAG3 inhibitor.

Embodiment 19. The method of Embodiment 18, wherein the LAG3 inhibitor is an anti-LAG3 antibody.

Embodiment 20. The method of Embodiment 19, wherein the anti-LAG3 antibody is GSK2831781.

Embodiment 21. The method of Embodiment 8, wherein the immune checkpoint inhibitor is a TIM3 inhibitor.

Embodiment 22. The method of Embodiment 21, wherein the TIM3 inhibitor is an anti-TIM3 antibody.

Embodiment 23. The method of any one of Embodiments 1-22, wherein the vinca alkaloid N-oxide is administered to the patient before the immune checkpoint inhibitor.

Embodiment 24. The method of any one of Embodiments 1-22, wherein the vinca alkaloid N-oxide is administered to the patient after the immune checkpoint inhibitor.

Embodiment 25. The method of any one of Embodiments 1-22, wherein the vinca alkaloid N-oxide is administered to the patient at the same time as the immune checkpoint inhibitor.

Embodiment 26. The method of any one of Embodiments 1-25, wherein the cancer is a solid tumor.

Embodiment 27. The method of any one of Embodiments 1-25, wherein the cancer is a hematological malignancy.

Embodiment 28. The method of any one of Embodiments 1-25, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

Embodiment 29. The method of Embodiment 28, wherein the cancer is selected from the group consisting of hepatocellular carcinoma, glioblastoma, lung cancer, breast cancer, head and neck cancer, prostate cancer, melanoma, and colorectal cancer.

Embodiment 30. The method of Embodiment 28, wherein the cancer is selected from the group consisting of non-small cell lung cancer, bladder cancer, head and neck cancer, ovarian cancer, and triple negative breast cancer.

Embodiment 31. The method of any one of Embodiments 1-30, wherein the cancer has become resistant to one or more conventional cancer treatments selected from the group consisting of radiotherapy, chemotherapy, hormonal therapy, or biologic therapy.

Embodiment 32. The method of Embodiment 31, wherein the cancer has become resistant to two or more conventional cancer treatments selected from the group consisting of radiotherapy, chemotherapy, hormonal therapy, or biologic therapy.

Embodiment 33. The method of Embodiments 31 or 32, wherein the cancer has become resistant to treatment with at least one immune checkpoint inhibitor.

Embodiment 34. The method of any one of Embodiments 1-33, wherein one or more of the biomarkers listed in Table 1 or Table 2 is differentially present in a biological sample taken from the patient as compared with a biological sample taken from a subject of another phenotypic status.

Embodiment 35. The method of Embodiment 34, wherein one or more of the biomarkers listed in Table 2 is differentially present in a biological sample taken from the patient as compared with a biological sample taken from a subject of another phenotypic status.

Embodiment 36. The method of Embodiment 35, wherein HIF-1α expression is differentially present in a sample taken from the patient as compared with a biological sample taken from a subject of another phenotypic status.

Embodiment 37. A kit comprising a vinca alkaloid N-oxide, or a pharmaceutically acceptable salt or solvate thereof, and instructions for administering the vinca alkaloid N-oxide, or a pharmaceutically acceptable salt or solvate thereof, together with an immune checkpoint inhibitor to a patient having cancer.

Embodiment 38. The kit of Embodiment 37, wherein the vinca alkaloid N-oxide is a N_{b′}-oxide, or a pharmaceutically acceptable salt or solvate thereof.

Embodiment 39. The kit of Embodiment 38, wherein the vinca alkaloid N-oxide is represented by a compound having Formula I:

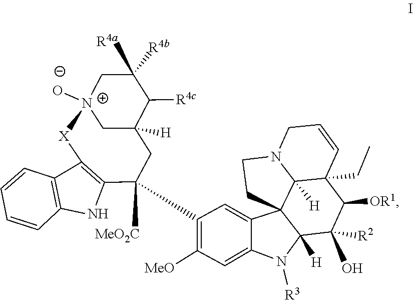

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 40. The kit of Embodiment 39, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 41. A lyophilized pharmaceutical composition comprising a vinca alkaloid N-oxide, or a pharmaceutically acceptable salt or solvate thereof, encapsulated in a liposome.

Embodiment 42. The lyophilized pharmaceutical composition of Embodiment 41, wherein the vinca alkaloid N-oxide is a N_{b′}-oxide, or a pharmaceutically acceptable salt or solvate thereof.

Embodiment 43. The lyophilized pharmaceutical composition of Embodiment 42, wherein the vinca alkaloid N-oxide is represented by a compound having Formula I:

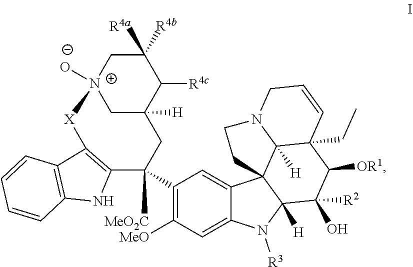

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 44. The lyophilized pharmaceutical composition of Embodiment 43, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 45. The lyophilized pharmaceutical composition of any one of Embodiments 41-44, wherein the liposome comprises sphingomyelin and cholesterol.

Embodiment 46. The lyophilized pharmaceutical composition of any one of Embodiments 41-44, wherein the liposome formulation comprises sphingomyelin, cholesterol, and 1,2-distearoyl-sn-glycero-3-phosphoethanolamine-N-[methoxy(polyethylene glycerol)-2000].

Embodiment 47. The lyophilized pharmaceutical composition of any one of Embodiments 41-46, wherein the composition is reconstituted in a sterile aqueous solution for parenteral administration to a patient.

Embodiment 48. The lyophilized pharmaceutical composition of Embodiment 47, wherein the sterile aqueous solution is water, saline, or 5% dextrose in water.

Embodiment 49. A kit comprising the lyophilized pharmaceutical composition of any one of Embodiments 41-46, and instructions for reconstituting the lyophilized pharmaceutical composition in a sterile aqueous solution for parenteral administration together with an immune checkpoint inhibitor to a patient having cancer.

Embodiment 50. The method of any one of claims 1-36, wherein the vinca alkaloid N-oxide and the immune checkpoint inhibitor are administered to the patient as separate pharmaceutical compositions.

Embodiment 51. A vinca alkaloid N-oxide, or a pharmaceutically acceptable salt thereof, for use in the treatment of cancer, in combination with an immune checkpoint inhibitor.

Embodiment 52. The vinca alkaloid N-oxide for use of Embodiment 51, wherein the vinca alkaloid N-oxide is a N_{b′}-oxide, or a pharmaceutically acceptable salt or solvate thereof.

Embodiment 53. The vinca alkaloid N-oxide for use of Embodiment 52, wherein the vinca alkaloid N-oxide is represented by a compound having Formula I:

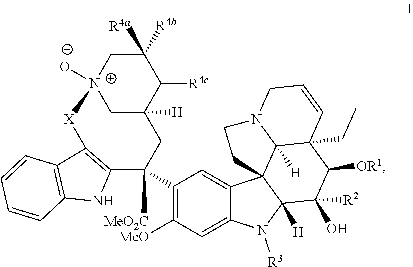

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 54. The vinca alkaloid N-oxide for use of Embodiment 53, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 55. The vinca alkaloid N-oxide for use of any one of Embodiments 51-54, wherein the vinca alkaloid N-oxide, or a pharmaceutically acceptable salt or solvate thereof, is administered to the patient encapsulated in a liposome.

Embodiment 56. The vinca alkaloid N-oxide for use of Embodiment 55, wherein the liposome comprises sphingomyelin and cholesterol.

Embodiment 57. The vinca alkaloid N-oxide for use of Embodiment 55, wherein the liposome comprises sphingomyelin, cholesterol, and 1,2-distearoyl-sn-glycero-3-phosphoethanolamine-N-[methoxy(polyethylene glycerol)-2000].

Embodiment 58. The vinca alkaloid N-oxide for use of any one of Embodiments 51-57, wherein immune checkpoint inhibitor is selected from the group consisting of a PD-1 inhibitor, a PD-L1 inhibitor, a CTLA-4 inhibitor, a LAG3 inhibitor, a TIGIT inhibitor, and a TIM3 inhibitor.

Embodiment 59. The vinca alkaloid N-oxide for use of Embodiment 58, wherein the immune checkpoint inhibitor is a PD-1 inhibitor.

Embodiment 60. The vinca alkaloid N-oxide for use of Embodiment 59, wherein the PD-1 inhibitor is an anti-PD-1 antibody.

Embodiment 61. The vinca alkaloid N-oxide for use of Embodiment 60, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 62. The vinca alkaloid N-oxide for use of Embodiment 58, wherein the immune checkpoint inhibitor is a PD-L1 inhibitor.

Embodiment 63. The vinca alkaloid N-oxide for use of Embodiment 62, wherein the PD-L1 inhibitor is an anti-PD-L1 antibody.

Embodiment 64. The vinca alkaloid N-oxide for use of Embodiment 63, wherein the anti-PD-L1 antibody is selected from the group consisting of avelumab, atezolizumab, durvalumab, and STI-A1014.

Embodiment 65. The vinca alkaloid N-oxide for use of Embodiment 58, wherein the immune checkpoint inhibitor is an anti-CTLA-4 inhibitor.

Embodiment 66. The vinca alkaloid N-oxide for use of Embodiment 65, wherein the anti-CTLA-4 inhibitor is an anti-CTLA-4 antibody.

Embodiment 67. The vinca alkaloid N-oxide for use of Embodiment 66, wherein the anti-CTLA-4 antibody is selected from the group consisting of ipilimumab and tremelimumab.

Embodiment 68. The vinca alkaloid N-oxide for use of Embodiment 58, wherein the immune checkpoint inhibitor is a LAG3 inhibitor.

Embodiment 69. The vinca alkaloid N-oxide for use of Embodiment 68, wherein the LAG3 inhibitor is an anti-LAG3 antibody.

Embodiment 70. The vinca alkaloid N-oxide for use of Embodiment 69, wherein the anti-LAG3 antibody is GSK2831781.

Embodiment 71. The vinca alkaloid N-oxide for use of Embodiment 58, wherein the immune checkpoint inhibitor is a TIM3 inhibitor.

Embodiment 72. The vinca alkaloid N-oxide for use of Embodiment 71, wherein the TIM3 inhibitor is an anti-TIM3 antibody.

Embodiment 73. The vinca alkaloid N-oxide for use of any one of Embodiments 51-72, wherein the vinca alkaloid N-oxide is administered to the patient before the immune checkpoint inhibitor.

Embodiment 74. The vinca alkaloid N-oxide for use of any one of Embodiment 51-72, wherein the vinca alkaloid N-oxide is administered to the patient after the immune checkpoint inhibitor.

Embodiment 75. The vinca alkaloid N-oxide for use of any one of Embodiments 51-72, wherein the vinca alkaloid N-oxide is administered to the patient at the same time as the immune checkpoint inhibitor.

Embodiment 76. The vinca alkaloid N-oxide for use of any one of Embodiments 51-75, wherein the cancer is a solid tumor.

Embodiment 77. The vinca alkaloid N-oxide for use of any one of Embodiments 51-75, wherein the cancer is a hematological malignancy.

Embodiment 78. The vinca alkaloid N-oxide for use of any one of Embodiments 51-75, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

Embodiment 79. The vinca alkaloid N-oxide for use of Embodiment 78, wherein the cancer is selected from the group consisting of hepatocellular carcinoma, glioblastoma, lung cancer, breast cancer, head and neck cancer, prostate cancer, melanoma, and colorectal cancer.

Embodiment 80. The vinca alkaloid N-oxide for use of Embodiment 78, wherein the cancer is selected from the group consisting of non-small cell lung cancer, bladder cancer, head and neck cancer, ovarian cancer, and triple negative breast cancer.

Embodiment 81. The vinca alkaloid N-oxide for use of any one of Embodiments 51-80, wherein the cancer has become resistant to one or more conventional cancer treatments selected from the group consisting of radiotherapy, chemotherapy, hormonal therapy, or biologic therapy.

Embodiment 82. The vinca alkaloid N-oxide for use of Embodiment 81, wherein the cancer has become resistant to two or more conventional cancer treatments selected from the group consisting of radiotherapy, chemotherapy, hormonal therapy, or biologic therapy.

Embodiment 83. The vinca alkaloid N-oxide for use of Embodiments 81 or 82, wherein the cancer has become resistant to treatment with at least one immune checkpoint inhibitor.

Embodiment 84. The vinca alkaloid N-oxide for use of any one of Embodiments 51-83, wherein one or more of the biomarkers listed in Table 1 or Table 2 is differentially present in a biological sample taken from the patient as compared with a biological sample taken from a subject of another phenotypic status.

Embodiment 85. The vinca alkaloid N-oxide for use of Embodiment 84, wherein one or more of the biomarkers listed in Table 2 is differentially present in a biological sample taken from the patient as compared with a biological sample taken from a subject of another phenotypic status.

Embodiment 86. The vinca alkaloid N-oxide for use of Embodiment 85, wherein HIF-1α expression is differentially present in a sample taken from the patient as compared with a biological sample taken from a subject of another phenotypic status.

Embodiment 87. Use of a vinca alkaloid N-oxide, or a pharmaceutically acceptable salt thereof, for the manufacture of medicament for use in combination therapy for treating cancer, wherein the medicament is to be administered in combination with an immune checkpoint inhibitor.

Embodiment 88. The use of Embodiment 87, wherein the vinca alkaloid N-oxide is a N_{b′}-oxide, or a pharmaceutically acceptable salt or solvate thereof.

Embodiment 89. The use of Embodiment 88, wherein the vinca alkaloid N-oxide is represented by a compound having Formula I:

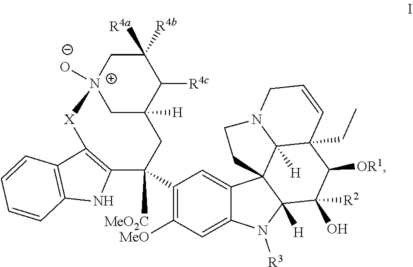

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 90. The use of Embodiment 89, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 91. The use of any one of Embodiments 87-90, wherein the vinca alkaloid N-oxide, or a pharmaceutically acceptable salt or solvate thereof, is administered to the patient encapsulated in a liposome.

Embodiment 92. The use of Embodiment 91, wherein the liposome comprises sphingomyelin and cholesterol.

Embodiment 93. The use of Embodiment 91, wherein the liposome comprises sphingomyelin, cholesterol, and 1,2-distearoyl-sn-glycero-3-phosphoethanolamine-N-[methoxy(polyethylene glycerol)-2000].

Embodiment 94. The use of any one of Embodiments 87-93, wherein immune checkpoint inhibitor is selected from the group consisting of a PD-1 inhibitor, a PD-L1 inhibitor, a CTLA-4 inhibitor, a LAG3 inhibitor, a TIGIT inhibitor, and a TIM3 inhibitor.

Embodiment 95. The use of Embodiment 94, wherein the immune checkpoint inhibitor is a PD-1 inhibitor.

Embodiment 96. The use of Embodiment 95, wherein the PD-1 inhibitor is an anti-PD-1 antibody.

Embodiment 97. The use of Embodiment 96, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 98. The use of Embodiment 94, wherein the immune checkpoint inhibitor is a PD-L1 inhibitor.

Embodiment 99. The use of Embodiment 98, wherein the PD-L1 inhibitor is an anti-PD-L1 antibody.

Embodiment 100. The use of Embodiment 99, wherein the anti-PD-L1 antibody is selected from the group consisting of avelumab, atezolizumab, durvalumab, and STI-A1014.

Embodiment 101. The use of Embodiment 94, wherein the immune checkpoint inhibitor is an anti-CTLA-4 inhibitor.

Embodiment 102. The use of Embodiment 101, wherein the anti-CTLA-4 inhibitor is an anti-CTLA-4 antibody.

Embodiment 103. The use of Embodiment 102, wherein the anti-CTLA-4 antibody is selected from the group consisting of ipilimumab and tremelimumab.

Embodiment 104. The use of Embodiment 94, wherein the immune checkpoint inhibitor is a LAG3 inhibitor.

Embodiment 105. The use of Embodiment 104, wherein the LAG3 inhibitor is an anti-LAG3 antibody.

Embodiment 106. The use of Embodiment 105, wherein the anti-LAG3 antibody is GSK2831781.

Embodiment 107. The use of Embodiment 94, wherein the immune checkpoint inhibitor is a TIM3 inhibitor.

Embodiment 108. The use of Embodiment 107, wherein the TIM3 inhibitor is an anti-TIM3 antibody.

Embodiment 109. The use of any one of Embodiments 87-108, wherein the vinca alkaloid N-oxide is administered to the patient before the immune checkpoint inhibitor.

Embodiment 110. The use of any one of Embodiment 87-108, wherein the vinca alkaloid N-oxide is administered to the patient after the immune checkpoint inhibitor.

Embodiment 111. The use of any one of Embodiments 87-108, wherein the vinca alkaloid N-oxide is administered to the patient at the same time as the immune checkpoint inhibitor.

Embodiment 112. The use of any one of Embodiments 87-111, wherein the cancer is a solid tumor.

Embodiment 113. The use of any one of Embodiments 87-111, wherein the cancer is a hematological malignancy.

Embodiment 114. The use of any one of Embodiments 87-111, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

Embodiment 115. The use of Embodiment 114, wherein the cancer is selected from the group consisting of hepatocellular carcinoma, glioblastoma, lung cancer, breast cancer, head and neck cancer, prostate cancer, melanoma, and colorectal cancer.

Embodiment 116. The use of Embodiment 114, wherein the cancer is selected from the group consisting of non-small cell lung cancer, bladder cancer, head and neck cancer, ovarian cancer, and triple negative breast cancer.

Embodiment 117. The use of any one of Embodiments 87-116, wherein the cancer has become resistant to one or more conventional cancer treatments selected from the group consisting of radiotherapy, chemotherapy, hormonal therapy, or biologic therapy.

Embodiment 118. The use of Embodiment 117, wherein the cancer has become resistant to two or more conventional cancer treatments selected from the group consisting of radiotherapy, chemotherapy, hormonal therapy, or biologic therapy.

Embodiment 119. The use of Embodiments 117 or 118, wherein the cancer has become resistant to treatment with at least one immune checkpoint inhibitor.

Embodiment 120. The use of any one of Embodiments 87-119, wherein one or more of the biomarkers listed in Table 1 or Table 2 is differentially present in a biological sample taken from the patient as compared with a biological sample taken from a subject of another phenotypic status.

Embodiment 121. The use of Embodiment 120, wherein one or more of the biomarkers listed in Table 2 is differentially present in a biological sample taken from the patient as compared with a biological sample taken from a subject of another phenotypic status.

Embodiment 122. The use of Embodiment 121, wherein HIF-1α expression is differentially present in a sample taken from the patient as compared with a biological sample taken from a subject of another phenotypic status.

Embodiment 123. A pharmaceutical composition comprising a vinca alkaloid N-oxide, or a pharmaceutically acceptable salt thereof, for the treatment of cancer in a patient, wherein the pharmaceutical composition is to be administered to the patient in combination with an immune checkpoint inhibitor.

Embodiment 124. The pharmaceutical composition of Embodiment 123, wherein the vinca alkaloid N-oxide is a N_{b′}-oxide, or a pharmaceutically acceptable salt or solvate thereof.

Embodiment 125. The pharmaceutical composition of Embodiment 124, wherein the vinca alkaloid N-oxide is represented by a compound having Formula I:

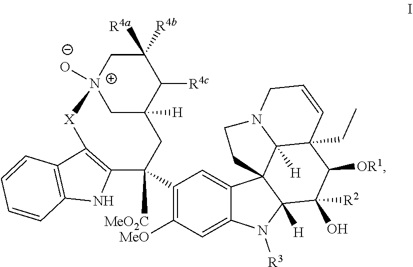

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 126. The pharmaceutical composition of Embodiment 125, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 127. The pharmaceutical composition of any one of Embodiments 123-126, wherein the vinca alkaloid N-oxide, or a pharmaceutically acceptable salt or solvate thereof, is administered to the patient encapsulated in a liposome.

Embodiment 128. The pharmaceutical composition of Embodiment 127, wherein the liposome comprises sphingomyelin and cholesterol.

Embodiment 129. The pharmaceutical composition of Embodiment 127, wherein the liposome comprises sphingomyelin, cholesterol, and 1,2-distearoyl-sn-glycero-3-phosphoethanolamine-N-[methoxy(polyethylene glycerol)-2000].

Embodiment 130. The pharmaceutical composition of any one of Embodiments 123-129, wherein immune checkpoint inhibitor is selected from the group consisting of a PD-1 inhibitor, a PD-L1 inhibitor, a CTLA-4 inhibitor, a LAG3 inhibitor, a TIGIT inhibitor, and a TIM3 inhibitor.

Embodiment 131. The pharmaceutical composition of Embodiment 130, wherein the immune checkpoint inhibitor is a PD-1 inhibitor.

Embodiment 132. The pharmaceutical composition of Embodiment 131, wherein the PD-1 inhibitor is an anti-PD-1 antibody.

Embodiment 133. The pharmaceutical composition of Embodiment 132, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment 134. The pharmaceutical composition of Embodiment 130, wherein the immune checkpoint inhibitor is a PD-L1 inhibitor.

Embodiment 135. The pharmaceutical composition of Embodiment 134, wherein the PD-L1 inhibitor is an anti-PD-L1 antibody.

Embodiment 136. The pharmaceutical composition of Embodiment 135, wherein the anti-PD-L1 antibody is selected from the group consisting of avelumab, atezolizumab, durvalumab, and STI-A1014.

Embodiment 137. The pharmaceutical composition of Embodiment 136, wherein the immune checkpoint inhibitor is an anti-CTLA-4 inhibitor.

Embodiment 138. The pharmaceutical composition of Embodiment 137, wherein the anti-CTLA-4 inhibitor is an anti-CTLA-4 antibody.

Embodiment 139. The pharmaceutical composition of Embodiment 138, wherein the anti-CTLA-4 antibody is selected from the group consisting of ipilimumab and tremelimumab.

Embodiment 140. The pharmaceutical composition of Embodiment 130, wherein the immune checkpoint inhibitor is a LAG3 inhibitor.

Embodiment 141. The pharmaceutical composition of Embodiment 140, wherein the LAG3 inhibitor is an anti-LAG3 antibody.

Embodiment 142. The pharmaceutical composition of Embodiment 141, wherein the anti-LAG3 antibody is GSK2831781.

Embodiment 143. The pharmaceutical composition of Embodiment 130, wherein the immune checkpoint inhibitor is a TIM3 inhibitor.

Embodiment 144. The pharmaceutical composition of Embodiment 143, wherein the TIM3 inhibitor is an anti-TIM3 antibody.

Embodiment 145. The pharmaceutical composition of any one of Embodiments 123-144, wherein the vinca alkaloid N-oxide is administered to the patient before the immune checkpoint inhibitor.

Embodiment 146. The pharmaceutical composition of any one of Embodiment 123-144, wherein the vinca alkaloid N-oxide is administered to the patient after the immune checkpoint inhibitor.

Embodiment 147. The pharmaceutical composition of any one of Embodiments 123-144, wherein the vinca alkaloid N-oxide is administered to the patient at the same time as the immune checkpoint inhibitor.

Embodiment 148. The pharmaceutical composition of any one of Embodiments 123-147, wherein the cancer is a solid tumor.

Embodiment 149. The pharmaceutical composition of any one of Embodiments 123-147, wherein the cancer is a hematological malignancy.

Embodiment 150. The pharmaceutical composition of any one of Embodiments 123-147, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

Embodiment 151. The pharmaceutical composition of Embodiment 150, wherein the cancer is selected from the group consisting of hepatocellular carcinoma, glioblastoma, lung cancer, breast cancer, head and neck cancer, prostate cancer, melanoma, and colorectal cancer.

Embodiment 152. The pharmaceutical composition of Embodiment 150, wherein the cancer is selected from the group consisting of non-small cell lung cancer, bladder cancer, head and neck cancer, ovarian cancer, and triple negative breast cancer.

Embodiment 153. The pharmaceutical composition of any one of Embodiments 123-152, wherein the cancer has become resistant to one or more conventional cancer treatments selected from the group consisting of radiotherapy, chemotherapy, hormonal therapy, or biologic therapy.

Embodiment 154. The pharmaceutical composition of Embodiment 153, wherein the cancer has become resistant to two or more conventional cancer treatments selected from the group consisting of radiotherapy, chemotherapy, hormonal therapy, or biologic therapy.

Embodiment 155. The pharmaceutical composition of Embodiments 153 or 154, wherein the cancer has become resistant to treatment with at least one immune checkpoint inhibitor.

Embodiment 156. The pharmaceutical composition of any one of Embodiments 123-155, wherein one or more of the biomarkers listed in Table 1 or Table 2 is differentially present in a biological sample taken from the patient as compared with a biological sample taken from a subject of another phenotypic status.

Embodiment 157. The pharmaceutical composition of Embodiment 156, wherein one or more of the biomarkers listed in Table 2 is differentially present in a biological sample taken from the patient as compared with a biological sample taken from a subject of another phenotypic status.

Embodiment 158. The pharmaceutical composition of Embodiment 157, wherein HIF-1α expression is differentially present in a sample taken from the patient as compared with a biological sample taken from a subject of another phenotypic status.

Embodiment 159. A method of treating a patient having cancer, the method comprising administering to the patient in need thereof a therapeutically effective amount of a vinca alkaloid N-oxide, or a pharmaceutically acceptable salt or solvate thereof, wherein one or more of the biomarkers listed in Table 2 is differentially present in a biological sample taken from the patient as compared with a biological sample taken from a subject of another phenotypic status.

Embodiment 160. The method of Embodiment 159, wherein the vinca alkaloid N-oxide is a N_{b′}-oxide, or a pharmaceutically acceptable salt or solvate thereof.

Embodiment 161. The method of Embodiment 160, wherein the vinca alkaloid N-oxide is represented by a compound having Formula I:

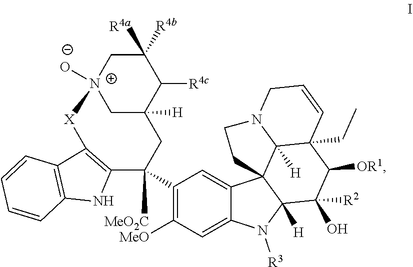

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment 162. The method of Embodiment 161, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment 163. The method of any one of Embodiments 159-162, wherein HIF-1α expression is differentially present in a sample taken from the patient as compared with a biological sample taken from a subject of another phenotypic status.

The disclosure provides the following particular embodiments.

Embodiment A 1. A method of treating a patient having cancer, the method comprising administering to the patient in need thereof a therapeutically effective amount of:
(a) a vinca alkaloid N-oxide, or a pharmaceutically acceptable salt or solvate thereof; and
(b) an immune checkpoint inhibitor.

Embodiment A 2. The method of Embodiment A 1, wherein the vinca alkaloid N-oxide is a N_{b′}-oxide, or a pharmaceutically acceptable salt or solvate thereof.

Embodiment A 3. The method of Embodiment A 2, wherein the vinca alkaloid N-oxide is represented by a compound having Formula I:

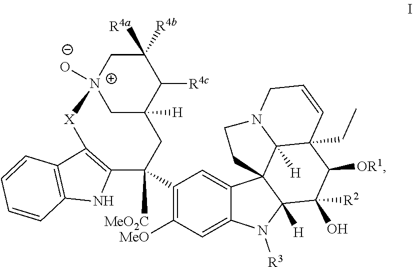

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment A 4. The method of Embodiment A 3, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment A 5. The method of any one of Embodiments A 1-A 4, wherein the vinca alkaloid N-oxide, or a pharmaceutically acceptable salt or solvate thereof, is administered to the patient encapsulated in a liposome.

Embodiment A 6. The method of Embodiment A 5, wherein the liposome comprises sphingomyelin and cholesterol.

Embodiment A 7. The method of Embodiment A 5, wherein the liposome comprises sphingomyelin, cholesterol, and 1,2-distearoyl-sn-glycero-3-phosphoethanolamine-N-[methoxy(polyethylene glycerol)-2000].

Embodiment A 8. The method of any one of Embodiments A 1-A 7, wherein immune checkpoint inhibitor is selected from the group consisting of a PD-1 inhibitor, a PD-L1 inhibitor, a CTLA-4 inhibitor, a LAG3 inhibitor, a TIM3 inhibitor, a VISTA inhibitor, a TIGIT inhibitor, and a cd47 inhibitor.

Embodiment A 9. The method of Embodiment A 8, wherein the immune checkpoint inhibitor is a PD-1 inhibitor.

Embodiment A 10. The method of Embodiment A 9, wherein the PD-1 inhibitor is an anti-PD-1 antibody.

Embodiment A 11. The method of Embodiment A 10, wherein the anti-PD-1 antibody is selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

Embodiment A 12. The method of Embodiment A 8, wherein the immune checkpoint inhibitor is a PD-L1 inhibitor.

Embodiment A 13. The method of Embodiment A 12, wherein the PD-L1 inhibitor is an anti-PD-L1 antibody.

Embodiment A 14. The method of Embodiment A 13, wherein the anti-PD-L1 antibody is selected from the group consisting of avelumab, atezolizumab, durvalumab, and STI-A1014.

Embodiment A 15. The method of Embodiment A 8, wherein the immune checkpoint inhibitor is an anti-CTLA-4 inhibitor.

Embodiment A 16. The method of Embodiment A 15, wherein the anti-CTLA-4 inhibitor is an anti-CTLA-4 antibody.

Embodiment A 17. The method of Embodiment A 16, wherein the anti-CTLA-4 antibody is selected from the group consisting of ipilimumab and tremelimumab.

Embodiment A 18. The method of Embodiment A 8, wherein the immune checkpoint inhibitor is a LAG3 inhibitor.

Embodiment A 19. The method of Embodiment A 18, wherein the LAG3 inhibitor is an anti-LAG3 antibody.

Embodiment A 20. The method of Embodiment A 19, wherein the anti-LAG3 antibody is GSK2831781.

Embodiment A 21. The method of Embodiment A 8, wherein the immune checkpoint inhibitor is a TIM3 inhibitor.

Embodiment A 22. The method of Embodiment A 21, wherein the TIM3 inhibitor is an anti-TIM3 antibody.

Embodiment A 23. The method of Embodiment A 8, wherein the immune checkpoint inhibitor is a VISTA inhibitor.

Embodiment A 24. The method of Embodiment A 23, wherein the VISTA inhibitor is an anti-VISTA antibody.

Embodiment A 25. The method of Embodiment A 8, wherein the immune checkpoint inhibitor is a cd47 inhibitor.

Embodiment A 26. The method of Embodiment A 25, wherein the cd47 inhibitor is an anti-cd47 antibody.

Embodiment A 27. The method of Embodiment A 8, wherein the immune checkpoint inhibitor is a TIGIT inhibitor.

Embodiment A 28. The method of Embodiment A 27, wherein the TIGIT inhibitor is an anti-TIGIT antibody.

Embodiment A 29. The method of any one of Embodiments A 1-A 28, wherein the vinca alkaloid N-oxide is administered to the patient before the immune checkpoint inhibitor.

Embodiment A 30. The method of any one of Embodiments A 1-A 28, wherein the vinca alkaloid N-oxide is administered to the patient after the immune checkpoint inhibitor.

Embodiment A 31. The method of any one of Embodiments A 1-A 28, wherein the vinca alkaloid N-oxide is administered to the patient at the same time as the immune checkpoint inhibitor.

Embodiment A 32. The method of any one of Embodiments A 1-A 31, wherein the cancer is a solid tumor.

Embodiment A 33. The method of any one of Embodiments A 1-A 31, wherein the cancer is a hematological malignancy.

Embodiment A 34. The method of any one of Embodiments A 1-A 31, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

Embodiment A 35. The method of Embodiment A 34, wherein the cancer is selected from the group consisting of hepatocellular carcinoma, glioblastoma, lung cancer, breast cancer, head and neck cancer, prostate cancer, melanoma, and colorectal cancer.

Embodiment A 36. The method of Embodiment A 34, wherein the cancer is selected from the group consisting of non-small cell lung cancer, bladder cancer, head and neck cancer, ovarian cancer, and triple negative breast cancer.

Embodiment A 37. The method of any one of Embodiments A 1-A 36, wherein the cancer has become resistant to one or more conventional cancer treatments selected from the group consisting of radiotherapy, chemotherapy, hormonal therapy, or biologic therapy.

Embodiment A 38. The method of Embodiment A 33, wherein the cancer has become resistant to two or more conventional cancer treatments selected from the group consisting of radiotherapy, chemotherapy, hormonal therapy, or biologic therapy.

Embodiment A 39. The method of Embodiments A 37 or A 38, wherein the cancer has become resistant to treatment with at least one immune checkpoint inhibitor.

Embodiment A 40. The method of any one of Embodiments A 1-A 39, wherein one or more of the biomarkers listed in Table 1 or Table 2 is differentially present in a biological sample taken from the patient as compared with a biological sample taken from a subject of another phenotypic status.

Embodiment A 41. The method of Embodiment A 40, wherein one or more of the biomarkers listed in Table 2 is differentially present in a biological sample taken from the patient as compared with a biological sample taken from a subject of another phenotypic status.

Embodiment A 42. The method of Embodiment A 41, wherein HIF-1α expression is differentially present in a sample taken from the patient as compared with a biological sample taken from a subject of another phenotypic status.

Embodiment A 43. A kit comprising a vinca alkaloid N-oxide, or a pharmaceutically acceptable salt or solvate thereof, and instructions for administering the vinca alkaloid N-oxide, or a pharmaceutically acceptable salt or solvate thereof, together with an immune checkpoint inhibitor to a patient having cancer.

Embodiment A 44. The kit of Embodiment A 43, wherein the vinca alkaloid N-oxide is a N_{b′}-oxide, or a pharmaceutically acceptable salt or solvate thereof.

Embodiment A 45. The kit of Embodiment A 44, wherein the vinca alkaloid N-oxide is represented by a compound having Formula I:

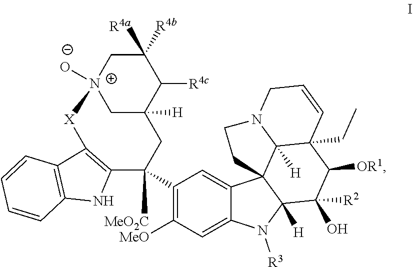

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment A 46. The kit of Embodiment A 45, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment A 47. A lyophilized pharmaceutical composition comprising a vinca alkaloid N-oxide, or a pharmaceutically acceptable salt or solvate thereof, encapsulated in a liposome.

Embodiment A 48. The lyophilized pharmaceutical composition of Embodiment A 47, wherein the vinca alkaloid N-oxide is a N_{b′}-oxide, or a pharmaceutically acceptable salt or solvate thereof.

Embodiment A 49. The lyophilized pharmaceutical composition of Embodiment A 48, wherein the vinca alkaloid N-oxide is represented by a compound having Formula I:

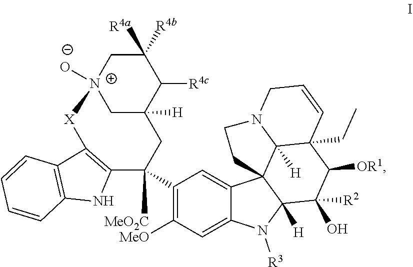

or a pharmaceutically acceptable salt or solvate thereof, wherein:
R¹ is selected from the group consisting of hydrogen and —C(═O)CH₃;
R² is selected from the group consisting of —C(═O)OCH₃ and —C(═O)NH₂;
R³ is selected from the group consisting of —CH₃ and —CHO;
R^{4a} is selected from the group consisting of hydrogen and —OH;
R^{4b} is selected from the group consisting of —CH₂CH₃ and —CF₂CH₃;
R^{4c} is hydrogen; or
R^{4a} and R^{4c} taken together form a double bond; and
X is selected from the group consisting of —CH₂— and —CH₂CH₂—.

Embodiment A 50. The lyophilized pharmaceutical composition of Embodiment A 49, wherein the vinca alkaloid N-oxide is selected from the group consisting of:
(a) vinblastine N_{b′}-oxide, or a pharmaceutically acceptable salt or solvate thereof,
(b) vincristine N_{b′}-oxide, or a pharmaceutically acceptable salt or solvate thereof,
(c) vindesine N_{b′}-oxide, or a pharmaceutically acceptable salt or solvate thereof,
(d) vinorelbine N_{b′}-oxide, or a pharmaceutically acceptable salt or solvate thereof, and
(e) vinflunine N_{b′}-oxide, or a pharmaceutically acceptable salt or solvate thereof.

Embodiment A 51. The lyophilized pharmaceutical composition of any one of Embodiments A 47-A 51, wherein the liposome comprises sphingomyelin and cholesterol.

Embodiment A 52. The lyophilized pharmaceutical composition of any one of Embodiments A 47-A 51, wherein the liposome comprises sphingomyelin, cholesterol, and 1,2-distearoyl-sn-glycero-3-phosphoethanolamine-N-[methoxy(polyethylene glycerol)-2000].

Embodiment A 53. The lyophilized pharmaceutical composition of any one of Embodiments A 47-A 52, wherein the composition is reconstituted in a sterile aqueous solution for parenteral administration to a patient.

Embodiment A 54. The lyophilized pharmaceutical composition of Embodiment A 53, wherein the sterile aqueous solution is water, saline, or 5% dextrose in water.

Embodiment A 55. A kit comprising the lyophilized pharmaceutical composition of any one of Embodiments A 47-A 52, and instructions for reconstituting the lyophilized pharmaceutical composition in a sterile aqueous solution for parenteral administration together with an immune checkpoint inhibitor to a patient having cancer.

Embodiment A 56. The method of any one of Embodiments A 1-A 42, wherein vinca alkaloid N-oxide is vinblastine N_{b′}-oxide, or a pharmaceutically acceptable salt or solvate thereof.

Embodiment A 57. The kit of any one of Embodiments A 43-A 46, wherein vinca alkaloid N-oxide is vinblastine N_{b′}-oxide, or a pharmaceutically acceptable salt or solvate thereof.

Embodiment A 58. The lyophilized pharmaceutical composition of any one of Embodiments A 47-A 54, wherein vinca alkaloid N-oxide is vinblastine N_{b′}-oxide, or a pharmaceutically acceptable salt or solvate thereof.

Embodiment A 59. The kit of Embodiment A 55, wherein vinca alkaloid N-oxide is vinblastine N_{b′}-oxide, or a pharmaceutically acceptable salt or solvate thereof.

### EXAMPLES

### Example 1

### Efficacy Evaluation of Vinblastine N_{b′}-Oxide (CPD100 Li) Alone or in Combination with Anti-mCTLA-4, Anti-mPD-L1, or Anti-mVISTA

The anti-cancer effects of CPD100 Li alone or in combination with anti-mCTLA-4, anti-mPD-L1, or anti-mVISTA against CT26.WT murine colon carcinoma in female BALB/c mice were evaluated. CPD100 Li is a liposomal formulation of vinblastine N_{b′}-oxide comprising sphingomyelin/cholesterol (55/45; mol/mol).

### Test Agents and Vehicles

Isotype Control (Clone MPC-11)

| Supplier | BioXCell | | Lot/Cat # | 645420M2/BP0086 | |
|---|---|---|---|---|---|
| Date received | NA | | Physical | Clear, colorless | |
| | | | description | solution | |
| Concentration | 4.11 | mg/mL | Storage | 4° | C. |

| Mol. Formula | NA | Mol wt/Form wt | NA | |
|---|---|---|---|---|
| Purity | NA | % Parent | NA | |
| Vehicle | Dulbecco's Phosphate | High Dose | 1 | mg/mL |
| | Buffered Saline (DPBS) | Formulation | | |
| Formulation | 7.4 | Stability | 7 | days |
| pH | | | | |
| Form. Desc. | Clear, colorless solution | Storage | 4° | C. |

| | | | | |
|---|---|---|---|---|
| Dose volume | 0.01 | mL/g | Dose expressed as | Vendor indicated, |
| | | | | uncorrected |

CPD100Li

| Supplier | Cascade Prodrug Inc. | Lot/Cat # | CPD100 | |
|---|---|---|---|---|
| Date received | Jul. 29, 2020 | Physical description | White powder | |
| Concentration | NA | Storage | 4° | C. |
| Mol. Formula | NA | Mol wt/Form wt | 827/827 | g/mole |
| Purity | 100% | % Parent | 100% | |
| Vehicle | Saline | High Dose | 3.75 | mg/mL |
| | | Formulation | | |
| Formulation | 5.3 | Stability | 1 | day |
| pH | | | | |
| Form. Desc. | powder | Storage | 4° | C. |

| | | | |
|---|---|---|---|
| Dose volume | 0.008 mL/g | Dose expressed as | Bulk, uncorrected |

| | |
|---|---|
| Comment | CPD100Li is a liposomal formulation of vinblastine N_{b′}-oxide comprising |
| | sphingomyelin/cholesterol (55/45; mol/mol). See Shah et al., Journal of |
| | Controlled Release 253: 37-45 (2017). |

Anti-mCTLa-4 (Clone 9D39)

| Supplier | BioXCell | | Lot/Cat # | 731719A2/BP0164 | |
|---|---|---|---|---|---|
| Date received | NA | | Physical | Clear, colorless | |
| | | | description | solution | |
| Concentration | 8.50 | mg/mL | Storage | 4° | C. |

| Mol. Formula | NA | Mol wt/Form wt | NA | |
|---|---|---|---|---|
| Purity | NA | % Parent | NA | |
| Vehicle | DPBS | High Dose | 1 | mg/mL |
| | | Formulation | | |
| Formulation | NA | Stability | 7 | days |
| pH | | | | |
| Form. Desc. | Clear, colorless | Storage | 4° | C. |

| | solution | | | |
|---|---|---|---|---|
| Dose volume | 0.01 | mL/g | Dose expressed as | Vendor indicated, |

uncorrected

Anti-mPD-L1 (Clone 10F.9G2)

| Supplier | BioXCell | | Lot/Cat # | 720619J3/BP0101 | |
|---|---|---|---|---|---|
| Date received | NA | | Physical | Clear, colorless | |
| | | | description | solution | |
| Concentration | 6.34 | mg/mL | Storage | 4° | C. |

| Mol. Formula | NA | Mol wt/Form wt | NA | |
|---|---|---|---|---|
| Purity | NA | % Parent | NA | |
| Vehicle | DPBS | High Dose | 1.175 | mg/mL |
| | | Formulation | | |
| Formulation | 7.1 | Stability | 7 | days |
| pH | | | | |
| Form. Desc. | Clear, colorless | Storage | 4° | C. |

| | solution | | | |
|---|---|---|---|---|
| Dose volume | 0.01 | mL/g | Dose expressed as | Vendor indicated, |

uncorrected

Anti-mVISTA (Clone 13F3)

| Supplier | BioXCell | | Lot/Cat # | 704218J3/BP0310 | |
|---|---|---|---|---|---|
| Date received | NA | | Physical | Clear, colorless | |
| | | | description | solution | |
| Concentration | 6.93 | mg/mL | Storage | 4° | C. |

| Mol. Formula | NA | Mol wt/Form wt | NA | |
|---|---|---|---|---|
| Purity | NA | % Parent | NA | |
| Vehicle | DPBS | High Dose | 1 | mg/mL |
| | | Formulation | | |
| Formulation pH | 7.3 | Stability | 7 | days |
| Form. Desc. | Clear, colorless | Storage | 4° | C. |

| | solution | | | |
|---|---|---|---|---|
| Dose volume | 0.01 | mL/g | Dose expressed as | Vendor indicated, |

uncorrected

### Animals and Husbandry

All procedures carried out in this experiment were conducted in compliance with the applicable laws, regulations and guidelines of the National Institutes of Health (NIH).

| Species | Mouse | | Source | Envigo | |
|---|---|---|---|---|---|
| Strain | BALB/c (BALB/cAnNHsd) | | Sex | Female | |
| Age at implant | 7-8 | weeks | On Study/Total | 104/182 | |
| Diet | Teklad 2918.15 Rodent Diet | | Water | Ad libitum | |
| Supplements | Hydrogel added to the cage | | Acclimation | 7 | days |
| | bottom when body weight | | | | |
| | loss exceeded 10%. | | | | |
| Housing | Innovive disposable | | Animals/cage | 5 or less | |
| | ventilated caging with corn | | | | |
| | cob bedding inside | | | | |
| | Biobubble ® Clean Rooms | | | | |
| Temp | 70 ± 2° | F. | Light cycle | 12/12 | hr |
| Humidity | 30-70% | | ID method | Ear punch | |
| Measure freq | 3/week | | Weigh freq | 3/week | |
| Staged by | Calipers | | Min individual | 16.7 | g |
| | | | weight (D 7) | | |
| Overall mean | 19.1 | g | Overall mean | 93 | mm³ |

| | | | |
|---|---|---|---|
| body weight | | tumor burden | |
| (D 7) | | (D 7) | |
| Necropsy | Yes | Health Checks | Daily |

| | |
|---|---|
| Euthanasia | Study termination, >2000 mm³ tumor burden, >20% body weight loss |
| criteria | and severe clinical signs. |

### Cell Preparation/Implantation

| | | | |
|---|---|---|---|
| Model | CT26.WT | Histotype | Murine colon |
| | | | carcinoma |
| Source | ATCC | Implant type | Cells |
| Media | RPMI 1640 Medium, 1 | Dissociation | 0.25% Trypsin/2.21 |
| | mM Na Pyruvate, 10 mM | solution | mM EDTA in HBSS |
| | HEPES buffer, 2.8 mL | | |
| | 45% glucose (1.25 g), | | |
| | 10% Non-Heat- | | |
| | Inactivated Fetal Bovine | | |
| | Serum (FBS) and 1% | | |
| | Penicillin/Streptomycin/L- | | |
| | Glutamine (PSG) | | |
| Route | Subcutaneous | Location | High right axilla |
| Inoculum | 5.00E+05 trypan- | Implant | Serum-free RPMI |
| | excluding cells | media | 1640 Medium |
| Matrigel | 0% | Inj. Volume | 200 μL |
| Viability | 80% | Viability | 78% |
| (pre) | | (post) | |

### Treatment

All mice were sorted into study groups based on caliper estimation of tumor burden. The mice were distributed to ensure that the mean tumor burden for all groups was within 10% of the overall mean tumor burden for the study population. Study groups were treated according to the schedule set forth in Table 3

TABLE 3

| | | | | | | | Days of |
|---|---|---|---|---|---|---|---|
| Group | N | Treatment | Dose | | ROA | Regimen | Treatment |
| 1 | 8 | Vehicle | 0.16 mL/20 g | | IV | Q14Dx2 | 7, 21 |
| | | isotype control | 10 | mg/kg | IP | (Q3Dx2, 10)(x2) | 7, 10, 21, 24 |
| | | (Clone MPC-11) | | | | | |
| 2 | 8 | CPD100 Li | 30 | mg/kg | IV | Q14Dx3 | 7, 21, 35 |
| 3 | 8 | CPD100 Li | 30 | mg/kg | IV | Q14Dx3 | 7, 21, 35 |
| | | isotype control | 10 | mg/kg | IP | (Q3Dx2, 10)(x2); | 7, 10, 21, 24, |
| | | (Clone MPC-11) | | | | QDx1 (D35) | 35 |
| 4 | 8 | anti-mCTLA-4 | 10 | mg/kg | IP | (Q3Dx2, 10)(x2) | 7, 10, 21, 24 |
| | | (Clone 9D9) | | | | | |
| 5 | 8 | anti-mPD-L1 | 10 | mg/kg | IP | Q3Dx2; QDx1 | 7, 10, 21 |
| | | (Clone 10F.9G2) | | | | (D21) | |
| 6 | 8 | anti-mVISTA | 10 | mg/kg | IP | Q3Dx2; QDx1 | 7, 10, 21 |
| | | (Clone 13F3) | | | | (D21) | |
| 7 | 8 | CPD100 Li | 30 | mg/kg | IV | Q14Dx3 | 7, 21, 35 |
| | | anti-mCTLA-4 | 10 | mg/kg | IP | (Q3Dx2, 10)(x2); | 7, 10, 21, 24, |
| | | (Clone 9D9) | | | | QDx1 (D35) | 35 |
| 8 | 8 | CPD100 Li | 30 | mg/kg | IV | Q14Dx2 | 7, 21 |
| | | anti-mPD-L1 | 10 | mg/kg | IP | Q3Dx2; QDx1 | 7, 10, 21 |
| | | (Clone 10F.9G2) | | | | (D21) | |
| 9 | 8 | CPD100 Li | 30 | mg/kg | IV | Q14Dx2 | 7, 21 |
| | | anti-mVISTA | 10 | mg/kg | IP | Q3Dx2; QDx1 | 7, 10, 21 |
| | | (Clone 13F3) | | | | (D21) | |

### Tumor Growth/General Observations/Controls

The mean estimated tumor burden for all groups in the experiment on the first day of treatment was 93 mm³, and all groups in the experiment were well-matched. All animals weighed at least 16.7 g at the initiation of therapy. Mean group body weights at first treatment were also well-matched, with an overall mean body weight of 19.1 g. Control animals experienced a 3.4 g (18.1%) mean weight gain during the treatment regimen. The median tumor volume doubling time for the Control Group was 2.6 days. There were no regressions in the Control Group.

A tumor burden of 2000 mm³ was chosen for evaluation of efficacy by time to progression. In the Control Group, the median time to progression was 14 days.

Efficacy evaluation was measured by median ΔT/ΔC on Day 20.

All thioglycolate cultures of cells used for implantation of this study were negative for gross bacterial contamination. All of this information is consistent with historical norms and the experiment was judged to be technically satisfactory and the data appropriate for evaluation.

### Miscellaneous

Day 0—The day on which the tumors are implanted (standard) or the day of first treatment.

Treatment Window—Begins with the first delivered dose and ends 2 weeks after the last treatment for each individual group.

### Efficacy

ΔC and ΔT—Are individual mouse endpoints that are calculated for each mouse as follows:

ΔT=Tₜ−T₀and ΔC=Cₜ−C₀,

Where Tₜ and T₀ are the tumor burdens of a treated mouse at time t or at the initiation of dosing, respectively. AC reflects similar calculations for the control mice.

Median ΔT/ΔC—Is a group endpoint. It is calculated for each day of treatment as:

$$Median \frac{Δ T}{Δ C} = ( \frac{Δ {T}_{m e d}}{Δ {C}_{m e d}} ) * 100 = ( \frac{median ( {T}_{t} - {T}_{0} )}{m e dian ( {C}_{t} - {C}_{0} )} ) * 100$$

The results are presented as a %. When the median ΔT/ΔC is negative (the median treated tumor burden is regressing), the median ΔT/ΔC is not reported and the Median % Regression is reported instead.

Tumor Growth Inhibition (TGI)—TGI is a group endpoint. The convention established by the NCI many years ago for calculation of this endpoint was followed. Tumor growth inhibition is calculated only when the median tumor burden is increasing (positive median ΔT). When the median tumor burden is regressing (negative median ΔT), the percent regression is calculated instead. TGI is calculated as follows:

$$\% T G I = ( 1 - \frac{Δ {T}_{m e d}}{Δ {C}_{m e d}} ) * 100$$

where ΔT_{med} is the median ΔT in the treated group, and ΔC_{med} is the median ΔC of the control group on any given day.

Time to Progression (TP)—Time to progression is an individual endpoint and can be used as a surrogate for lifespan or time on study. The selected tumor evaluation size is tumor model and study dependent. TP data is analyzed by Kaplan Meier methods just as traditional lifespan data. The Time to Progression for an individual animal is the number of days between initiation of treatment and death or the day that the animal reaches a selected evaluation size. The initiation of treatment is the day of first treatment in the study as a whole and is not specific to the group in question. Time to progression is a log-linear interpolation between the adjacent data points on either side of the selected tumor evaluation size. This normalizes the evaluation criteria for all animals.

If animals do not reach the selected evaluation size and is euthanized or found dead due to disease progression or lack of treatment tolerance, lifespan is reported instead of Time to Progression. Animals euthanized or found dead for causes unrelated to disease progression (technical errors, etc.) are excluded from this calculation and reported as “NA”. The median Time to Progression for a group is used to calculate the % Increase in Time to Progression (% ITP).

% Increase in Time to Progression (% ITP)—% ITP is a group endpoint. It is calculated as:

$$\% I T P = \{ \frac{[ ( median Treated TP ) - ( median Control TP ) ]}{median Control TP} \} * 100$$

Tumor Doubling Time (Td)—Td is an individual and group parameter, typically expressed as the median Td of the group. It is measured in days. Td can be calculated from any type of volumetric data (caliper measurements, BLI signals, etc). For QC purposes it is calculated for the exponential portion of the tumor growth curve. Data points during any lag phase and in the Gompertzian advanced stage are not included. Typical tumor burden limits are between 100 and 1000 mm³, but actual selection is data driven. Td is calculated for each mouse from a least square best fit of a log/linear plot of tumor burden vs day as:

Td=log 2/slope

On rare occasions the median Td is used as a potential indicator of efficacy. As such it is calculated as the median for every group, over a specified range of days thought to reflect a period of response to therapy.

### Tumor Regression

Complete Regression (CR)—An animal is credited a complete regression if its tumor burden is reduced to an immeasurable volume at any point after the first treatment. Our convention is to record any tumor volume measurement less than 63 mm3 as a “0”. The CR must be maintained for at least 2 consecutive measurements. This is in keeping with the convention of the NCI and reflects the inherent mechanical error in such measurements in addition to the biology of what is measured at those small sizes.

### (Individual Efficacy Parameter)

Partial Regression—An animal is credited with a partial regression if its tumor burden decreases to less than half of the tumor burden at first treatment. The PR must be maintained for at least 2 consecutive measurements for caliper driven studies. (For BLI driven studies the required confirmation is waived because of the dynamic range of the measurements and typically longer intervals between imaging.) PRs are tabulated exclusive of CRs, thus an animal that achieves a CR is not also counted as a PR.

### (Individual Efficacy Parameter)

Tumor-Free Survivor (TFS)—A TFS is any animal that (1) survives until termination of the study, and (2) has no reliably measurable evidence of disease at study termination. Mice that are tumor-free at some point during the study but are then euthanized for sampling or other purposes prior to the end of the study are not considered TFS. They are excluded from calculation of the % TFS.

### Results

The mean tumor volume curves of Groups 1-9 are provided in FIG. 1. The mean body weight change curves of Groups 1-9 are provided in FIG. 2. A summary of these results is provided in Table 4.

Combination treatment with CPD100 Li+anti-mCTLA-4 (Group 7) produced surprising anti-cancer activity in the CT26.WT (colon carcinoma) model, resulting in a Day 20 median ΔT/ΔC value of 4%, an increase in time to progression of >207%, and a 62.5% incidence of complete tumor regressions with 25.0% remaining as tumor-free survivors at the end of the study.

TABLE 4

| | Weight | Deaths In | Median | Increased | | | |
|---|---|---|---|---|---|---|---|
| | Change in | Treatment | ΔT/ΔC | Time to | Partial | Complete | Tumor Free |
| | Treatment | Window | Day 20 | Progression | Regression | Regression | Survivors |
| Group # | Window (%) | (%) | (%) | (%) | (%) | (%) | (%) |
| 1 | 18.1 | 0.0 | NA | NA | 0.0 | 0.0 | 0.0 |
| 2 | −7.5 | 12.5 | 56 | 128 | 0.0 | 0.0 | 0.0 |
| 3 | −8.5 | 37.5 | 60 | 71 | 0.0 | 0.0 | 0.0 |
| 4 | 13.0 | 0.0 | 55 | 35 | 0.0 | 0.0 | 0.0 |
| 5 | 12.1 | 25.0 | 54 | 42 | 0.0 | 0.0 | 0.0 |
| 6 | 8.1 | 25.0 | 59 | 21 | 0.0 | 0.0 | 0.0 |
| 7 | −14.0 | 25.0 | 4 | >207 | 0.0 | 62.5 | 25.0 |
| 8 | −13.6 | 87.5 | 30 | 7 | 0.0 | 0.0 | 0.0 |
| 9 | −8.7 | 87.5 | 29 | 7 | 0.0 | 0.0 | 0.0 |

### Example 2

A clinical study compares progression-free or overall survival using pembrolizumab or nivolumab to pembrolizumab or nivolumab in combination with vinblastine N_{b′}-oxide for participants with cancer who are untreated or have progressed after prior therapy. Participants will be randomized to receive either standard anti-PD-1 therapy plus placebo or standard anti-PD-1 therapy plus vinblastine N_{b′}-oxide.

Primary Outcome Measures: Progression-Free-Survival (PFS) and/or Overall Survival (OS)

Secondary Outcome Measures: Overall Response Rate (ORR) and/or Response Duration

### Eligibility

Ages Eligible for Study: Generally—18 Years and older
Genders Eligible for Study: Both

### Inclusion Criteria:

Histologically or cytologically confirmed diagnosis of cancer not amenable to local therapy
Must consent to allow correlative studies; must provide a newly obtained tissue/biopsy specimen (or specimen obtained within 60 days of consenting)
Radiographically measurable disease
Eastern Cooperative Oncology Group Performance Status of 0 or 1
Patient may have cancer with overexpressed HIF.

### Exclusion Criteria:

Chemotherapy, radiation therapy, or biological therapy within four weeks prior to the first dose of study drug, or not recovered from the AEs due to cancer therapies administered more than four weeks earlier
Participating or has participated in a study of an investigational agent or using an investigational device within 30 days of the first dose of study drug
Expected to require any other form of systemic or localized antineoplastic therapy while on study
Chronic systemic steroid therapy within two weeks before the planned date for first dose randomized treatment or on any other form of immunosuppressive medication
Known history of any other than the current malignancy excepting adequately treated basal or squamous cell carcinoma of the skin, superficial bladder cancer, in situ cervical cancer, breast cancer, or other in situ cancers
Known active central nervous system (CNS) metastases and/or carcinomatous meningitis
Active autoimmune disease or a documented history of autoimmune disease or syndrome that requires systemic steroids or immunosuppressive agents
Prior treatment with any other anti-programmed cell death (PD) agent
Active infection requiring systemic therapy
Known history of Human Immunodeficiency Virus (HIV)
Active Hepatitis B or Hepatitis C
Regular user (including recreational use of) illicit drugs or had a recent history (within the last year) of substance abuse (including alcohol)
Pregnant or breastfeeding, or expecting to conceive or father children within the projected duration of the study.

### Protocols:

A first group of patients receive 2-10 mg/kg pembrolizumab (or flat dose equivalent) administered by intravenous infusion every three weeks and vinblastine N_{b′}-oxide administered orally or by IV at 0.01-100 mg once weekly. Vinblastine N_{b′}-oxide administration is started 1-14 days prior to initiating pembrolizumab therapy and, optionally, continues on the day of pembrolizumab administration, and, optionally, continues until disease progression or until vinblastine N_{b′}-oxide therapy is no longer beneficial. The control patients receive 2-10 mg/kg pembrolizumab (or flat dose equivalent) administered by intravenous infusion every three weeks.

A second group of patients receive 3 mg/kg nivolumab administered over 60 minutes by intravenous infusion every 2 weeks and vinblastine N_{b′}-oxide administered orally or by IV at 0.01-100 mg once weekly. Vinblastine N_{b′}-oxide administration is started 1-14 days prior to prior to initiating nivolumab therapy, continues on the day of nivolumab administration, and, optionally, continues until disease progression or until vinblastine N_{b′}-oxide therapy is no longer beneficial. The control patients receive 3 mg/kg nivolumab administered over 60 minutes by intravenous infusion every 2 weeks.

### Example 3

### Open Label Phase 2 Study Assessing the Combination of Checkpoint Blockade Immunotherapy and Vinblastine N_{b′}-Oxide in Patients Relapsing from or Refractory to Standard Anti-PD-1 Therapy

Primary Endpoint: ORR

Secondary Endpoints: PFS, OS, Duration of Response, Safety

### Inclusion Criteria:

Histologically confirmed diagnosis of cancer not amenable to local therapy
Eastern Cooperative Oncology Group (ECOG) performance status of 0 or 1
At least one measurable lesion
Adequate organ function
Prior therapy with an anti-PD-1 or anti-PD-L1 antibody
Patient has disease with overexpressed MYC and/or MCL1

### Exclusion Criteria:

Chemotherapy, targeted small molecule therapy, radiotherapy, or biological cancer therapy (including monoclonal antibodies) within 4 weeks prior to the first dose of trial treatment, or not recovered (<=Grade 1 or baseline) from adverse events due to a previously administered agent.
Expected to require any other form of systemic or localized antineoplastic therapy while in study.
Known active central nervous system (CNS) metastases and/or carcinomatous meningitis.
Documented history of clinically severe autoimmune disease, or a syndrome that requires systemic steroids or immunosuppressive agents.
Receiving systemic steroid therapy or any other form of immunosuppressive therapy within 1 week prior to the first dose of study treatment.
Received a live vaccine within 4 weeks prior to the first dose of trial treatment.
History or evidence of active pneumonitis.
Human immunodeficiency virus (HIV)-positive.
Active Hepatitis B or C.
Pregnant, breastfeeding, or expecting to conceive or father children within the projected duration of the trial treatment through 120 days after the last dose of study medication.

### Dosing Protocol:

TABLE 5
Vinblastine N_{b′}-oxide + CheckPoint Inhibitor
Combination Dosing & Schedules

| | Every 2 weeks | Every 3 weeks | Every 4 weeks |
|---|---|---|---|
| Pembrolizumab | X | X | |
| 2 mg/kg | | | |
| Pembrolizumab | X | X | |
| 10 mg/kg | | | |
| Pembrolizumab | X | X | |
| 200 mg | | | |
| Pembrolizumab | X | X | |
| 300 mg | | | |
| Nivolumab | X | X | X |
| 3 mg/kg | | | |
| Nivolumab | X | X | X |
| 1 mg/kg | | | |
| Pidilizumab | X | X | X |
| 3 mg/kg | | | |
| Pidilizumab | X | X | X |
| 1.5 mg/kg | | | |
| STI-A1110 | X | X | X |
| 2 mg/kg | | | |
| STI-A1110 | X | X | X |
| 2 mg/kg | | | |
| Durvalumab | X | X | |
| 10 mg/kg | | | |
| Durvalumab | X | X | |
| 2 mg/kg | | | |
| Durvalumab | | X | X |
| 15, g/kg | | | |
| Avelumab | X | X | X |
| 1200 mg | | | |
| Avelumab | X | X | X |
| 10 mg/kg | | | |
| Avelumab | X | X | X |
| 5 mg/kg | | | |
| Atezolizumab | | X | |
| 1200 mg | | | |
| STI-A1014 | X | X | X |
| 10 mg/kg | | | |
| STI-A1014 | X | X | X |
| 15 mg/kg | | | |

* vinblastine N_{b′}-oxide is dosed weekly (0.1-100 mg/kg) starting 1-14 day prior to initiating checkpoint inhibitor therapy and continuing until disease progression or investigator decision

### Results

Combining vinblastine N_{b′}-oxide with at least one checkpoint inhibitor in patients may reverse immune evasion and induce clinically relevant responses in patients previously nonresponding to or failing checkpoint inhibitor therapy or de novo cancer patients. Objective responses are associated with lack of tumor progression and extension of long term survival compared to historical controls using (the antibody) alone. In one embodiment, patients receiving vinblastine N_{b′}-oxide and an immune checkpoint inhibitor achieve an extension of time to progression (or progression-free survival) of at least 2 months, at least 4 months, at least 6 months, at least 8 months, at least 10 months or at least 12 months. In another embodiment, at least some of the patients receiving vinblastine N_{b′}-oxide and an immune checkpoint inhibitor achieve an extension of duration of response of at least 2 months, at least 4 months, at least 6 months, at least 8 months, at least 10 months or at least 12 months.

### Example 4

### Placebo-Controlled, Randomized Phase 2 Study of Pembrolizumab Plus Vinblastine N_{b′}-Oxide Vs. Pembrolizumab+Placebo in Participants with Previously-Treated Locally Advanced Unresectable or Metastatic Colorectal Cancer

Primary Endpoint: PFS

Secondary Endpoint: ORR, Duration of Response

### Inclusion Criteria:

Histologically-proven locally advanced unresectable or metastatic high colorectal carcinoma
Previously treated with at least two lines of approved standard therapies, which must include fluoropyrimidine, oxaliplatin, irinotecan, bevacizumab, and cetuximab or panitumumab
Eastern Cooperative Oncology Group performance status of 0 or 1
Patient has disease that, optionally, overexpresses HIF
Life expectancy of greater than 3 months
At least one measureable lesion
Female participants of childbearing potential should be willing to use 2 methods of birth control or be surgically sterile, or abstain from heterosexual activity for the course of the study through 120 days after the last dose of study medication
Male participants should agree to use an adequate method of contraception starting with the first dose of study therapy through 120 days after the last dose of study medication
Adequate organ function

### Exclusion Criteria:

Currently participating in another study and receiving trial treatment, participated in a study of an investigational agent and received trial treatment within 4 weeks of the first dose of medication in this study, or used an investigational device within 4 weeks of the first dose of medication in this study
Active autoimmune disease that has required systemic treatment in past 2 years
Diagnosis of immunodeficiency or receiving systemic steroid therapy or any other form of immunosuppressive therapy within 7 days prior to the first dose of study medication
Known active central nervous system (CNS) metastases and/or carcinomatous meningitis
Prior monoclonal antibody (mAb), chemotherapy, targeted small molecule therapy, or radiation therapy within 2 weeks prior to study Day 1 or not recovered (i.e., ≤Grade 1 or at baseline) from adverse events due to a previously administered agent
Prior therapy with an anti-programmed cell death (PD)-1, anti-PD-L1, or anti-PD-L2 agent, or participant has previously participated in Merck pembrolizumab (MK-3475) clinical trial
Known additional malignancy that is progressing or requires active treatment with the exception of basal cell carcinoma of the skin or squamous cell carcinoma of the skin that has undergone potentially curative therapy, or in situ cervical cancer
Received a live vaccine within 30 days of planned start of study medication
Known history of human immunodeficiency virus (HIV)
Known active Hepatitis B or C
Known history or any evidence of interstitial lung disease or active, non-infectious pneumonitis
Active infection requiring systemic therapy
Known psychiatric or substance abuse disorders that would interfere with cooperation with the requirements of the trial
Pregnant or breastfeeding, or expecting to conceive or father children within the projected duration of the trial, starting with the screening visit through 120 days after the last dose of trial medication

### Dosing Protocol:

Patients receive 2-10 mg/kg pembrolizumab administered by intravenous infusion every three weeks and vinblastine N_{b′}-oxide administered orally or IV 1-7 days prior to pembrolizumab administration and, optionally, on the day of pembrolizumab administration, and, optionally, continuously thereafter until disease progression or until it is no longer beneficial. The control patients receive 2 mg/kg pembrolizumab administered by intravenous infusion every three weeks.

### Results:

When used in patients with tumors overexpressing HIF, vinblastine N_{b′}-oxide combined with pembrolizumab provides better clinical activity than pembrolizumab alone in the same patients. Objective responses are associated with lack of tumor progression and extension of long term survival compared to historical controls using (the antibody) alone. In one embodiment, patients receiving vinblastine N_{b′}-oxide and pembrolizumab achieve an extension of time to progression (or progression-free survival) of at least 2 months, at least 4 months, at least 6 months, at least 8 months, at least 10 months or at least 12 months. In another embodiment, at least some of the patients receiving vinblastine N_{b′}-oxide and pembrolizumab achieve an extension of duration of response of at least 2 months, at least 4 months, at least 6 months, at least 8 months, at least 10 months or at least 12 months.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A method of treating a patient having cancer, the method comprising administering to the patient in need thereof a therapeutically effective amount of:
(a) a vinca alkaloid N-oxide, or a pharmaceutically acceptable salt or solvate thereof, and
(b) one or more immune checkpoint inhibitors;
wherein the vinca alkaloid N-oxide, or a pharmaceutically acceptable salt or solvate thereof is:
(i) vinblastine N_{b′}-oxide, or a pharmaceutically acceptable salt or solvate thereof, (ii) vincristine N_{b′}-oxide, or a pharmaceutically acceptable salt or solvate thereof; (iii) vindesine N_{b′}-oxide, or a pharmaceutically acceptable salt or solvate thereof; (iv) vinorelbine N_{b′}-oxide, or a pharmaceutically acceptable salt or solvate thereof, or (v) vinflunine N_{b′}-oxide, or a pharmaceutically acceptable salt or solvate thereof; and
the one or more immune checkpoint inhibitors comprise an anti-PD-1 antibody, an anti-PD-L1 antibody, an anti-CTLA-4 antibody, an anti-LAG3 antibody, an anti-TIM3 antibody, an anti-VISTA antibody, an anti-TIGIT antibody, or an anti-cd47 antibody, or a combination thereof.

2. The method of claim 1, wherein the vinca alkaloid N-oxide, or a pharmaceutically acceptable salt or solvate thereof, is administered to the patient encapsulated in a liposome.

3. The method of claim 2, wherein the liposome comprises sphingomyelin and cholesterol.

4. The method of claim 2, wherein the liposome comprises sphingomyelin, cholesterol, and 1,2-distearoyl-sn-glycero-3-phosphoethanolamine-N-[methoxy(polyethylene glycerol)-2000].

5. The method of claim 1, wherein the immune checkpoint inhibitor is an anti-PD-1 antibody selected from the group consisting of nivolumab, pembrolizumab, pidilizumab, STI-A1110, PDR001, MEDI-0680, AGEN2034, BGB-A317, AB122, TSR-042, PF-06801591, cemiplimab, SYM021, JNJ-63723283, HLX10, LZM009, and MGA012.

6. The method of claim 1, wherein the immune checkpoint inhibitor is an anti-PD-L1 antibody selected from the group consisting of avelumab, atezolizumab, durvalumab, and STI-A1014.

7. The method of claim 1, wherein the immune checkpoint inhibitor is an anti-CTLA-4 antibody selected from the group consisting of ipilimumab and tremelimumab.

8. The method of claim 1, wherein the immune checkpoint inhibitor is an anti-LAG3 antibody that is GSK2831781.

9. The method of claim 1, wherein the immune checkpoint inhibitor is an anti-TIM3 antibody.

10. The method of claim 1, wherein the immune checkpoint inhibitor is an anti-VISTA antibody.

11. The method of claim 1, wherein the immune checkpoint inhibitor is an anti-cd47 antibody.

12. The method of claim 1, wherein the immune checkpoint inhibitor is an anti-TIGIT antibody.

13. The method of claim 1, wherein the cancer selected from the group consisting of adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatocellular carcinoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

14. The method of claim 1, wherein HIF-1α expression is differentially present in a sample taken from the patient as compared with a biological sample taken from a subject of another phenotypic status.

15. A lyophilized pharmaceutical composition comprising a vinca alkaloid N-oxide, or a pharmaceutically acceptable salt or solvate thereof, encapsulated in a liposome, wherein the vinca alkaloid N-oxide, or a pharmaceutically acceptable salt or solvate thereof is (i) vinblastine N_{b′}-oxide, or a pharmaceutically acceptable salt or solvate thereof, (ii) vincristine N_{b′}-oxide, or a pharmaceutically acceptable salt or solvate thereof; (iii) vindesine N_{b′}-oxide, or a pharmaceutically acceptable salt or solvate thereof; (iv) vinorelbine N_{b′}-oxide, or a pharmaceutically acceptable salt or solvate thereof; or (v) vinflunine N_{b′}-oxide, or a pharmaceutically acceptable salt or solvate thereof.

16. The lyophilized pharmaceutical composition of claim 15, wherein the liposome comprises sphingomyelin and cholesterol.

17. The lyophilized pharmaceutical composition of claim 15, wherein the liposome comprises sphingomyelin, cholesterol, and 1,2-distearoyl-sn-glycero-3-phosphoethanolamine-N-[methoxy(polyethylene glycerol)-2000].

18. The lyophilized pharmaceutical composition of claim 15, wherein the composition is reconstituted in a sterile aqueous solution for parenteral administration to a patient.

19. The lyophilized pharmaceutical composition of claim 18, wherein the sterile aqueous solution is water, saline, or 5% dextrose in water.

20. A kit comprising the lyophilized pharmaceutical composition of claim 15, and instructions for reconstituting the lyophilized pharmaceutical composition in a sterile aqueous solution for parenteral administration together with an immune checkpoint inhibitor to a patient having cancer.
